SEQUENCE LISTING (44 sequences)

SEQ ID NO: 1 - DNA, 99 bp, synthetic construct
  source 1..99: mol_type = other DNA
taatacgactcactatagggcgaattgggtaccgggcccagaccacaacggtttccctctagaaataattttgtttaactttaagaaggagatatacat

SEQ ID NO: 2 - DNA, 99 bp, synthetic construct
  source 1..99: mol_type = other DNA
taatccgactcactatagggcgaattgggtaccgggcccagaccacaacggtttccctctagaaataattttgtttaactttaagaaggagatatacat

SEQ ID NO: 3 - DNA, 99 bp, synthetic construct
  source 1..99: mol_type = other DNA
gaatacgactcactatagcgcgaattgggtaccgggcccagaccacaacggtttccctctagaaataattttgtttaactttaagaaggagatatacat

SEQ ID NO: 4 - protein, 348 aa, synthetic construct
MAVYFVTGKLGSGKTLVSVGKIQDKIVAGCKIATNLDLRLQNLPQVGRFAKTPRVLRIPDKPSISDLLAIGRGNDSYDENKNGLLVLDECGTWFNTRSWNDKERQPIIDWFLHARKLGWDIIFLVQDLSIVDKQARSALAEHVVYCRRLDRITLPFVGTLYSLITGSKMPLPKLHVGVVKYGDSQLSPTVERWLYTGKNLYNAYDTKQAFSSNYDSGVYSYLTPYLSHGRYFKPLNLGQKMKLTKIYLKKFSRVLCLAIGFASAFTYSYITQPKPEVKKVVSQTYDFDKFTIDSSQRLNLSYRYVFKDSKGKLINSDDLQKQGYSLTYIDLCTVSIKKGNSNEIVKCN

SEQ ID NO: 5 - protein, 410 aa, synthetic construct
MIDMLVLRLPFIDSLVCSRLSGNDLIAFVDLSKIATLSGMNLSARTVEYHIDGDLTVSGLSHPFESLPTHYSGIAFKIYEGSKNFYPCVEIKASPAKVLQGHNVFGTTDLALCSEALLLNFANSLPCLYDLLDVNATTISRIDATFSARAPNENIAKQVIDHLRNVSNGQTKSTRSQNWESTVTWNETSRHRTLVAYLKHVELQHQIQQLSSKPSAKMTSYQKEQLKVLSNPDLLEFASGLVRFEARIKTRYLKSFGLPLNLFDAIRFASDYNSQGKDLIFDLWSFSFSELFKAFEGDSMNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 6 - protein, 340 aa, synthetic construct
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGS

SEQ ID NO: 7 - protein, 426 aa, synthetic construct
MKLLNVINFVFLMFVSSSSFAQVIEMNNSPLRDFVTWYSKQSGESVIVSPDVKGTVTVYSSDVKPENLRNFFISVLRANNFDMVGSIPSIIQKYNPNNQDYIDELPSSDNQEYDDNSAPSGGFFVPQNDNVTQTFKINNVRAKDLIRVVELFVKSNTSKSSNVLSIDGSNLLVVSAPKDILDNLPQFLSTVDLPTDQILIEGLIFEVQQGDALDFSFAAGSQRGTVAGGVNTDRLTSVLSSAGGSFGIFNGDVLGLSVRALKTNSHSKILSVPRILTLSGQKGSISVGQNVPFITGRVTGESANVNNPFQTIERQNVGISMSVFPVAMAGGNIVLDITSKADSLSSSTQASDVITNQRSIATTVNLRDGQTLLLGGLTDYKNTSQDSGVPFLSKIPLIGLLFSSRSDSNEESTLYVLVKATIVRAL

SEQ ID NO: 8 - protein, 87 aa, synthetic construct
MIKVEIKPSQAQFTTRSGVSRQGKPYSLNEQLCYVDLGNEYPVLVKITLDEGQPAYAPGLYTVHLSSFKVGQFGSLMIDRLRLVPAK

SEQ ID NO: 9 - protein, 112 aa, synthetic construct
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDK

SEQ ID NO: 10 - protein, 33 aa, synthetic construct
MEQVADFDTIYQAMIQISVVLCFALGIIAGGQR

SEQ ID NO: 11 - protein, 73 aa, synthetic construct
MKKSLVLKASVAVATLVPMLSFAAEGDDPAKAAFNSLQASATEYIGYAWAMVVVIVGATIGIKLFKKFTSKAS

SEQ ID NO: 12 - protein, 32 aa, synthetic construct
MSVLVYSFASFVLGWCLRSGITYFTRLMETSS

SEQ ID NO: 13 - protein, 111 aa, synthetic construct
MNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 14 - protein, 108 aa, synthetic construct
MKLTKIYLKKFSRVLCLAIGFASAFTYSYITQPKPEVKKVVSQTYDFDKFTIDSSQRLNLSYRYVFKDSKGKLINSDDLQKQGYSLTYIDLCTVSIKKGNSNEIVKCN

SEQ ID NO: 15 - DNA, 1278 bp, synthetic construct
  source 1..1278: mol_type = other DNA
taatacgactcactatagggcgaattgggtaccgggcccagaccacaacggtttccctctagaaataattttgtttaactttaagaaggagatatacatatggctgtttattttgtaacaggaaaactaggctccggcaagaccttagtcagcgttggtaagatccaggataagatcgtggccggttgtaaaattgccaccaacctggatcttcgcctgcaaaacctgccacaggttggccgttttgcgaaaaccccgcgtgttctgcgtattccggataagccgagcatcagcgacttgctggcgatcggtcgcggcaacgactcgtacgacgagaataaaaacggtctgctggtgttggacgagtgcggtacatggttcaacacccgttcttggaatgataaagaaagacaaccgatcattgactggttcctgcacgctcgcaaattgggttgggatatcatctttctggtccaggacctgtcaatcgtggacaaacaagcacgttccgcgctggctgaacatgtggtgtactgccgtcgtctggaccgtatcaccctgccgttcgtgggcaccctgtactctctgattacgggtagcaagatgccgctgccgaaactgcacgtcggtgtggttaagtatggtgatagccagctttccccgaccgtcgagcgctggctgtataccggtaagaatctttacaatgcatatgatacgaagcaagcgtttagctctaactatgactccggtgtttattcgtacctcactccgtatttgagccatggtcgttatttcaaaccgctgaacttgggccagaaaatgaaattgaccaaaatttatctgaaaaaattcagccgtgttctttgcttagcgatcggcttcgcaagcgcgtttacgtacagctacattacgcagccgaaacccgaagttaaaaaagttgtaagccagacctacgactttgataagttcaccattgactcctcccaacgtctgaatctgtcgtaccgctacgtgtttaaggatagtaagggcaagctcatcaacagcgatgatttacagaagcaaggttatagcttgacctacattgacttgtgtaccgtttctatcaaaaagggaaacagcaacgagattgtgaagtgcaattaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 16 - DNA, 1464 bp, synthetic construct
  source 1..1464: mol_type = other DNA
taatacgactcactatagggcgaattgggtaccgggcccagaccacaacggtttccctctagaaataattttgtttaactttaagaaggagatatacatatgatagatatgttagtattgaggctacccttcatcgacagtcttgtgtgctcacgtttgtcgggaaacgatctgatcgcgttcgtggacttgtctaaaattgctactctgtctggtatgaacctgagcgcgcgtactgtagagtaccatattgatggtgacttgaccgttagcggcctgtcccacccgtttgaaagcttgccgacccattatagcggcatcgcgttcaaaatctacgaaggttccaagaacttttacccgtgtgttgagattaaggcgtcccccgcaaaagttctgcaaggtcacaacgtctttggtacgaccgatctcgccctctgtagcgaagcactgctgctaaactttgctaatagtttaccgtgcctgtatgacttgctggacgtgaatgcaaccaccatcagccgcattgatgcgacgtttagcgcgcgcgcaccgaatgaaaatatcgcgaaacaggttatcgaccacctgcgtaacgttagcaacggtcagaccaaaagcacccgttctcaaaactgggaaagcactgtcacctggaatgaaacctcccgtcatcgtaccctggtcgcatatttgaagcacgtggagctgcaacatcagattcaacagttgtcctctaagccgagcgctaagatgaccagctatcagaaggaacagttgaaggtgttgagcaatccggacctgttggaatttgcgagtggcctggtgcgtttcgaggcgcgcatcaaaaccagatacctgaagagctttggcctgccactgaacctgttcgatgcgattcgttttgcgtctgattacaactcgcaaggtaaagatttaatctttgatttatggagcttctccttcagcgagctattcaaagccttcgagggtgactctatgaatatctatgatgatagcgcggtcctggacgccatccaaagcaaacactttacgattaccccgagcggcaagacctcgttcgccaaggctagccgctacttcggtttttaccgccgtctggttaacgagggctacgactcagttgcactgaccatgccgcgtaactccttctggcgttacgtgtccgctctggtggagtgcggcattccgaaaagccagctgatgaacctgtctacgtgcaacaacgttgttccactcgtgcgcttcattaatgtggacttttcctctcagcgtccggactggtataacgagccggttctgaaaatcgcgtaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 17 - DNA, 1506 bp, synthetic construct
  source 1..1506: mol_type = other DNA
taatacgactcactatagggcgaattgggtaccgggcccagaccacaacggtttccctctagaaataattttgtttaactttaagaaggagatatacatatgaaaaagttattgtttgctatacccctagtcgttccgttttatagccacagcgcggaaaccgtggaatcttgtctggcgaaaccgcatacggaaaactcttttaccaatgtgtggaaagatgataagaccctggatagatatgcgaactatgagggttgtctttggaatgcgaccggcgtggtggtgtgcaccggcgacgaaacccaatgttatggtacttgggttccgattggtttggcgatcccggagaacgagggcgggggctctgagggcggcggtagcgagggtggcggttccgagggtggaggtaccaaaccgccggaatacggtgacaccccgatcccgggttatacgtacatcaacccgttggacggcacgtacccgccaggtactgagcagaatccggcaaacccaaatccgagcctggaagagagccaaccgttgaataccttcatgtttcagaacaaccgcttccgcaaccgtcagggtgcgctgaccgtttataccggtacagttacccagggtacggatccggtgaaaacctactatcagtacactccggtcagctcgaaagccatgtacgatgcgtattggaacggtaagttccgcgattgcgccttccacagcggcttcaacgaagatccgttcgtgtgcgaataccaaggtcagtcgagcgacctgccgcagccgcccgttaatgctggtggcgggagcggtggcggtagtggtggcggcagcgaaggcggtggatccgaagggggcggttccgagggcggtggctcggagggtggtggctctggcggtggctctggctccggcgacttcgattacgaaaaaatggcgaacgcgaacaagggtgctatgaccgagaacgcggatgaaaacgctctgcagagcgatgcaaagggtaagctggacagcgttgcgactgactacggcgccgcaattgacggcttcatcggcgacgttagcggtttggctaacggcaatggtgcaacgggtgactttgcgggctccaatagccaaatggcacaggttggtgacggtgacaacagcccgctgatgaataactttcgtcaatacctcccaagcctgccgcaatccgtggaatgccgtccgttcgtattctccgcgggtaagccttatgagttctcaatcgactgcgataagattaacctgtttcgtggtgtttttgcgtttctgctgtacgtggccacctttatgtatgtctttagcaccttcgccaacattttacgtaataaagagagctaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 18 - DNA, 1497 bp, synthetic construct
  source 1..1497: mol_type = other DNA
taatacgactcactatagggcgaattgggtaccgggcccagaccacaacggtttccctctagaaataattttgtttaactttaagaaggagatatacatatgaaactattaaatgttataaactttgtattcctgatgtttgtttcgtcctcatctttcgcccaggttattgagatgaataactccccgctgcgtgattttgtcacttggtatagcaagcagagcggcgaaagcgtcattgtttccccggatgttaaaggcaccgttaccgtttatagcagcgacgttaagccggaaaatctgcgcaacttcttcatcagcgtgctacgggcgaataacttcgatatggttggtagcatcccgagcattattcagaaatacaatccgaataaccaagattacattgacgaactgccgagctcggacaaccaagagtatgatgataacagcgctccgtccggtggtttttttgtaccgcagaatgataacgtaacgcagacgtttaaaatcaacaatgtgcgcgcgaaagatctgatccgtgtcgtggaactgttcgtgaagtctaacacgagcaagagctctaacgtgttgtctatcgacggctccaacttattagttgtttccgcgccaaaagacatcttggacaacctgcctcagtttctgtccaccgtggacctgccgaccgaccagattctgattgagggcttgatttttgaagttcagcaaggtgacgcattggacttcagcttcgcggcgggcagccaacgcggtacagttgctggtggcgtgaacaccgatcgtttgaccagcgttctctctagcgcggggggttcattcggcatcttcaacggtgatgtcttgggtctgtcggttcgtgccctgaaaaccaattctcacagcaagatcctgtccgtgccgcgtattctgaccctgtcaggacaaaaaggttccatctccgtgggccaaaacgtcccatttatcacgggtcgtgtgaccggcgagagcgcgaacgttaacaacccgttccagaccatcgagcgccagaacgtgggtattagcatgtccgtgttcccggtagcaatggcaggcggtaatattgtgctggacatcacgagtaaggcggattcgctgagctcttcgactcaggcaagcgacgtcatcaccaaccaaagaagcatcgccaccaccgttaatctgcgtgatggtcaaaccctgctgctgggcggtttgactgactacaaaaatacctcgcaagacagcggcgtgccgtttttgtcaaagatcccgctgataggcctgttgtttagcagccgttctgacagtaatgaagagagcactctttacgtgctggttaaggctaccattggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 19 - DNA, 495 bp, synthetic construct
  source 1..495: mol_type = other DNA
taatacgactcactatagggcgaattgggtaccgggcccagaccacaacggtttccctctagaaataattttgtttaactttaagaaggagatatacatatgattaaagtagaaataaagccctcacaagcgcagttcacgacccgttccggtgttagccgtcagggtaagccgtatagcttaaatgagcagctgtgctacgtggacctgggcaacgagtacccggttttggtcaaaatcaccctggacgaaggccaaccggcttacgcaccgggtctgtataccgtgcacctgagctcttttaaagttggtcaattcggctcgctgatgattgatcgtttgcgcctcgtgccagcgaagtaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 20 - DNA, 570 bp, synthetic construct
  source 1..570: mol_type = other DNA
taatacgactcactatagggcgaattgggtaccgggcccagaccacaacggtttccctctagaaataattttgtttaactttaagaaggagatatacatatgccagtactgttaggaatacccctattgttacgttttttgggtttcttgctggtcaccctgttcggctacttgctcacgtttctgaaaaaaggttttggcaagatcgcgatcgccatcagcctgttcctggcactgattatcggcctgaatagcattctggttggttacctgagcgacatctctgcgcagctgccgtcggacttcgtgcaaggtgttcaactgattctgccgtccaacgctttgccgtgcttctatgttattttgtccgtgaaggcggcgattttcatctttgatgtaaagcagaagatcgtgagctatcttgactgggataaataaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 21 - DNA, 333 bp, synthetic construct
  source 1..333: mol_type = other DNA
taatacgactcactatagggcgaattgggtaccgggcccagaccacaacggtttccctctagaaataattttgtttaactttaagaaggagatatacatatggaacaagtagctgattttgacacaatataccaggcaatgattcagattagcgttgtgttgtgcttcgctctgggcatcatcgcgggtggtcaacgttaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 22 - DNA, 453 bp, synthetic construct
  source 1..453: mol_type = other DNA
taatacgactcactatagggcgaattgggtaccgggcccagaccacaacggtttccctctagaaataattttgtttaactttaagaaggagatatacatatgaaaaagtcactagtattaaaagctagtgtcgctgtggccacgttggttccgatgctgtcgttcgccgcggaaggtgatgacccggcaaaagctgcgtttaacagcctgcaggcgtctgctaccgagtacatcggctatgcgtgggcaatggtggtggttattgttggtgcgaccattggcatcaagctgtttaaaaagttcaccagcaaggcgtcctaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 23 - DNA, 330 bp, synthetic construct
  source 1..330: mol_type = other DNA
taatacgactcactatagggcgaattgggtaccgggcccagaccacaacggtttccctctagaaataattttgtttaactttaagaaggagatatacatatgagtgtactagtttattcatttgcttctttcgtgctgggttggtgcttgcgctccggcatcacctactttacccgtctgatggaaaccagcagctaaggatccggctgctaacaaagcccgaaaggaagctgagttggctgctgccaccgctgagcaataactagcataaccccttggggcctctaaacgggtcttgaggggttttttgacaaagagggcgagcgttaa

SEQ ID NO: 24 - DNA, 8669 bp, synthetic construct
  source 1..8669: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggacggatcgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgccccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccctgcaggtctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagt

SEQ ID NO: 25 - DNA, 223 bp, synthetic construct
  source 1..223: mol_type = other DNA
gggctagtcaatgataattacatgtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactc

SEQ ID NO: 26 - DNA, 255 bp, synthetic construct
  source 1..255: mol_type = other DNA
tttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttc

SEQ ID NO: 27 - DNA, 255 bp, synthetic construct
  source 1..255: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 28 - DNA, 255 bp, synthetic construct
  source 1..255: mol_type = other DNA
ttgagatcctttttttctgcgcgtaatctgctgcttgcaaacaaaaaaaccaccgctatcaacggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 29 - DNA, 255 bp, synthetic construct
  source 1..255: mol_type = other DNA
ttgagatcctttttttctgcgcgtaatctgctgcttgcaaacaaaaaaaccaccgctaccaacggtggtttgtttgccggatcaagagctatcaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 30 - DNA, 255 bp, synthetic construct
  source 1..255: mol_type = other DNA
ttgagatcctttttttctgcgcgtaatctgctgcttgcaaacaaaaaaaccaccgctatcaacggtggtttgtttgccggatcaagagctatcaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 31 - DNA, 255 bp, synthetic construct
  source 1..255: mol_type = other DNA
ttgagatcctttttttctgcgcgtaatctgctgcttgcaaacaaaaaaaccaccgctatcagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 32 - DNA, 255 bp, synthetic construct
  source 1..255: mol_type = other DNA
ttgagatcctttttttctgcgcgtaatctgctgcttgcaaacaaaaaaaccaccgctaccagcggtggtttgtttgccggatcaagagctatcaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 33 - DNA, 255 bp, synthetic construct
  source 1..255: mol_type = other DNA
ttgagatcctttttttctgcgcgtaatctgctgcttgcaaacaaaaaaaccaccgctatcagcggtggtttgtttgccggatcaagagctatcaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 34 - DNA, 738 bp, synthetic construct
  source 1..738: mol_type = other DNA
atgacgttaactgcttcatcttcttcccgcgctgttacgaattctcctgtggttgttgcccttgattatcataatcgtgatgacgcgctggcctttgtcgacaagatcgacccacgcgattgtcgtctgaaggtcggcaaagagatgtttacattgtttgggccacagtttgtgcgcgaacttcaacagcgtggttttgatatctttcttgacctgaaattccacgatatccccaacactgcagcgcacgctgtcgctgctgcagctgacttaggcgtgtggatggtgaatgttcatgcctctggtggggcgcgtatgatgaccgcagcgcgtgaggcactggttccgtttggcaaagatgcaccgcttttgattgctgtgacagtgttgaccagcatggaagccagcgacctggtcgatcttggcatgacactgtcacctgcagattatgcagaacgtctggcggcactgacgcaaaaatgtggccttgatggtgtggtgtgttctgctcaggaagctgtgcgctttaaacaggtattcggtcaggagttcaaactggttacgccgggcattcgtccgcaggggagtgaagctggtgaccagcgccgcattatgacgccagaacaggcgttgtcggctggtgttgattatatggtgattggtcgcccggtaacgcaatcggtagatccagcgcagacgctgaaagcgatcaacgcctctttacagcggagtgcatga

SEQ ID NO: 35 - DNA, 86 bp, synthetic construct
  source 1..86: mol_type = other DNA
ttaatttcaactttaatcatacctcattaccttatgcgattttaagaactggctcattataccagtcaggacgttgggaagaaaaa

SEQ ID NO: 36 - DNA, 85 bp, synthetic construct
  source 1..85: mol_type = other DNA
atcgcataaggtaatgaggtatgattaaagttgaaattaaaccatctcaagcccaatttactactcgttctggtgtttctcgtca

SEQ ID NO: 37 - DNA, 132 bp, synthetic construct
  source 1..132: mol_type = other DNA
atttttcttcccaacgtcctgactggtataatgagccagttcttaaaatcgcataaggtaattcacaatgattaaagttgaaattaaaccatctcaagcccaatttactactcgttctggtgtttctcgtca

SEQ ID NO: 38 - DNA, 132 bp, synthetic construct
  source 1..132: mol_type = other DNA
tgacgagaaacaccagaacgagtagtaaattgggcttgagatggtttaatttcaactttaatcattgtgaattaccttatgcgattttaagaactggctcattataccagtcaggacgttgggaagaaaaat

SEQ ID NO: 39 - DNA, 34 bp, synthetic construct
  source 1..34: mol_type = other DNA
cttcggaaaaagagttgatagctcttgatccggc

SEQ ID NO: 40 - DNA, 41 bp, synthetic construct
  source 1..41: mol_type = other DNA
gcaaacaaaccaccgctgatagcggtggtttttttgtttgc

SEQ ID NO: 41 - DNA, 41 bp, synthetic construct
  source 1..41: mol_type = other DNA
gcaaacaaaaaaaccaccgctatcagcggtggtttgtttgc

SEQ ID NO: 42 - DNA, 34 bp, synthetic construct
  source 1..34: mol_type = other DNA
gccggatcaagagctatcaactctttttccgaag

SEQ ID NO: 43 - DNA, 90 bp, synthetic construct
  source 1..90: mol_type = other DNA
tgcaaacaaaaaaaccaccgctaccagcggtggtttgtttgccggatcaagagctaccaactctttttccgaaggtaactggcttcagca

SEQ ID NO: 44 - DNA, 90 bp, synthetic construct
  source 1..90: mol_type = other DNA
tgctgaagccagttaccttcggaaaaagagttggtagctcttgatccggcaaacaaaccaccgctggtagcggtggtttttttgtttgca

(19) United States
(12) Patent Application Publication
(10) Pub. No.: US 2026/0275325 A1
(43) Pub. Date: Sep. 17, 2026

(54) ENGINEERED CSSDNA PRODUCTION HOSTS AND PHAGEMIDS

(71) Applicant: Kano Therapeutics Inc., Cambridge, MA (US)

(72) Inventors: Gregory Benjamin FOLEY, Somerville, MA (US); Floris Alaska Silvia ENGELHARDT, Boston, MA (US); Beaven Carlyle ANKNER-EDELSTEIN, Cambridge, MA (US)

(73) Assignee: Kano Therapeutics Inc., Cambridge, MA (US)

(21) Appl. No.: 19/471,342

(22) PCT Filed: Mar. 29, 2024
(86) PCT No.: PCT/US2024/022394

§ 371 Date: Sep. 30, 2025

Related U.S. Application Data
(60) Provisional application No. 63/493,492, filed on Mar. 31, 2023.
(60) Provisional application No. 63/605,225, filed on Dec. 1, 2023.

(51) Int. Cl. C12N 15/10; C12N 15/85

(52) U.S. Cl. CPC C12N 15/10; C12N 15/85

(57) ABSTRACT

Described herein are engineered bacterial production strains that include engineered phagemids for the efficient production of high quality circular, single-stranded DNA (cssDNA). Also, described herein are DNA molecules that can be used to efficiently produce high quality cssDNA. Accordingly, methods of producing high quality cssDNA are also described.

### CROSS-REFERENCE TO RELATED APPLICATIONS

This application is a national stage application under 35 U.S.C. § 371 of International Application No. PCT/US2024/022394, filed internationally on Mar. 29, 2024, which claims priority from U.S. Provisional Patent Application No. 63/493,492, filed Mar. 31, 2023, and U.S. Provisional Patent Application No. 63/605,225, filed Dec. 1, 2023, the contents of each of which are herein incorporated by reference in their entirety.

### BACKGROUND

High fidelity cssDNA (circular single-stranded DNA) and ssDNA (single-stranded DNA) of custom sequence at greater than 1 kb is difficult to manufacture at scale through enzymatic or chemical processes.

### REFERENCE TO AN ELECTRONIC SEQUENCE LISTING

The content of the electronic sequence listing (304832000300seqlist.xml; Size: 61,620 bytes; and Date of Creation: Sep. 29, 2025) is herein incorporated by reference in its entirety.

### SUMMARY

Herein are described improvements in the development and production of ssDNA (single-stranded DNA) and cssDNA (circular single-stranded DNA).

Among other things, the present disclosure describes production strains that include a phagemid and at least two phage coding sequences incorporated into the genome of the production strain. In some production strains, at least 3, 4, 5, 6, 7, 8, 9, 10 or 11 phage coding sequences can be included in the genome of the production strain. Incorporation of coding sequences into the genome of the production strain offers improvements including decreasing the likelihood of producing replication-competent phages, and/or alleviating problems of batch-to-batch inconsistencies due to decreasing the reliance on helper virions and helper plasmids. Alternatively or additionally, this approach can reduce the metabolic burden on the production host by eliminating the need to maintain an extrachromosomally-replicating helper plasmid traditionally maintained through the addition of antibiotics to the medium.

Also described herein are cssDNA production systems that do not contain non-endogenous antibiotic resistance genes. In some embodiments the production strain and the phagemid included within the production strain are both free from non-endogenous antibiotic resistance coding sequences. One of ordinary skill in the art understands that the inclusion of antibiotic resistance coding sequences in recombinant DNA constructs aids in many molecular biology techniques. However, the present disclosure appreciates certain disadvantages of such an approach. For example, inclusion of such sequences may not be desirable for environmental and human safety reasons. Also, there is a metabolic cost to the production host of expressing the antibiotic resistance sequence and a cost of being exposed to antibiotic drugs even when the strain is resistant to the drugs. In certain embodiments, cssDNA production systems provided by the present disclosure do not utilize antibiotic resistance coding sequences in the production strain.

In certain embodiments, production strains provided herein include strains that include a phagemid that has a packaging signal, a designed sequence, and at least one selectable sequence that is not an antibiotic resistance gene and is selected from a sequence encoding an auxotrophic marker, an antitoxin, an RNA that inhibits the expression of a gene that would retard or stop growth of the bacterium if it were expressed in the absence of the RNA, a transcription factor repressor that inhibits the expression of a gene that would retard or stop growth of the bacterium if it were expressed in the absence of the transcription factor, a transcriptional activator that activates such a repressor, or a sequence that expresses a tRNA that associates with an unnatural amino acid that the production strain is engineered to require. In many instances the production strain itself is genomically altered to complement the activity of the selection sequence in the phagemid so that survival of the production strain without the presence of the phagemid is not possible.

Suitable production strains include strains susceptible to infection by a single-stranded, filamentous bacteriophages of the realm of Monodnaviria that are capable of being engineered and that can support phagemid replication. Bacteria from the families Enterobacteriaceae, Pseudomonadaceae, Spirillaceae, Xanthomonadaceae, Clostridium, and Propionibacterium are potentially useful production strains. One of ordinary skill in the art will appreciate that E. coli strains are particularly useful as production strains.

Exemplary phages that may be useful as a source of phage proteins include M13, Ff, Fd, Enterobacteria phage F1 [EF068134], Enterobacteria phage ID2, Enterobacteria phage NL95 [AF059243], Enterobacteria phage SP [X07489], Enterobacteria phage TW28, Enterobacteria phage Qbeta, Enterobacteria phage Qβ [AY099114], Enterobacteria phage M11 [AF059242], Enterobacteria phage ST, Enterobacteria phage TW18 [FJ483840], and Enterobacteria phage VK or a functional equivalent thereof. Packaging signals and other control sequences from phages can be used to make phagemids as described herein. In some embodiments, a genome of a particular phage, except for the packaging signal and the phage origin of replication (hereinafter ori) may be genomically integrated into the production strain. In such embodiments, a helper plasmid or helper phage will not be necessary and a phage genome will not be packaged into a phage capsid because the phage genome lacks the phage ori and packaging signal.

In some embodiments, genomically integrated coding sequences for phage proteins may be characterized by their activity as compared to their native activity when they are expressed from the control sequences found in the wildtype phage. When a control sequence is derived from a source that is different from the source of the coding sequence to which it is operably linked in an expression cassette, that control sequence can be referred to as a non-native control sequence, a non-endogenous control sequence or an engineered control sequence. The control sequence that is found operably linked to the coding sequence in an organism that is the source of the coding sequence can be referred to as the native control sequence or endogenous control sequence. One of ordinary skill in the art will appreciate that activity can be altered by altering the amino acid sequence of the protein, such as through truncation of the sequence or amino acid residue substitution. Activity can also be altered by increasing the level of expression of the protein through any means known in the art for overexpression. Genomically integrated phage protein sequences can be engineered to be differentially expressed as compared to each other, as well as compared to their native expression. Differentially expressed includes not only increasing or decreasing the activity of the protein but also includes temporally expressing one phage protein at a different time than another phage protein to optimize cssDNA production.

In some embodiments, a production strain includes genomically integrated phage coding sequences that encode at least two of the following M13 phage proteins (or analogs thereof): p1, p2, p3, p4, p5, p6, p7, p8, p9, p10 and p11 (see FIG. 1, panels A and B). In some embodiments, genomically integrated sequences can be overexpressed as compared to their native expression and/or the activity of one or more of the proteins can be altered. In particular embodiments, protein overexpression can be through the use of promoters, such as synthetic promoters, inducible promoter systems or promoters known to provide high levels of expression.

In some embodiments, activity of one or more phage proteins can be decreased as compared to their native activity. For example, in some embodiments, a production strain can include a p3 and/or a p5 phage protein that has less activity as compared to its native expression. In an exemplary embodiment described herein, decrease in activity can be accomplished through the use of truncations of p3 or p5.

As further described herein, selection sequences can be chosen from sequences that either are in RNA form, or translated into a protein form, and/or make up for a deficiency or sensitivity in the production strain. In some instances, in order for a selection sequence in a phagemid to work, a production strain described herein will need to be genetically engineered to render the production strain deficient or sensitive. In some embodiments, the lambda red recombineering system of genomic modification described in Example 1, below, can be used to achieve modification of the production strain as described herein.

In particular embodiments, when a selection sequence is a toxin/antitoxin system, a selection sequence can be selected from ccdB/ccdA, hokA/sokA, pemK/pemI, mazF/mazE, ChpBK ChpBI, relE/relB, parE/parD, hipA/hipB, or other toxin/antitoxin systems where the toxin is expressed from the host genome and the antitoxin is expressed from the phagemid. In certain embodiments when the selection sequence is an RNAi that down regulates a counter selectable sequence in the production strain, an RNAi sequence can be selected to interfere with the translation of HSVtk, Ura3, tetA, sacB, rpsL, pheS, pheS*, pheS**, thyA, lacY, gata-1, ccdB, hokA, pemK, mazF, chpBK, relE, parE, hipA or other counterselectable markers or toxins. Alternatively or additionally, in some embodiments, selection sequences may include or be selected from transcriptional repressors which can be used to downregulate the transcription of a deleterious sequence in the production strain. In some embodiments, transcriptional repressor sequences can be selected from tetR, araC, lacI, xylS or other sequences that reduce the expression of a counterselectable marker or toxin. Analogously, in some embodiments, a selection sequence can be chosen from any transcriptional activator known in the art. In some embodiments, a transcriptional activator can be used to increase the transcription level of a gene needed for the production strain to survive. Examples of transcriptional activators include araC, and xylR.

Particularly useful selection sequences to be used in methods and production systems described herein include auxotrophy sequences which are sequences that encode a protein that is needed by the production strain host for proper cellular function. In some embodiments that utilize such sequences, a production strain host is engineered to need the product of the selection sequence for optimum growth. A particularly useful selection sequence is the pyrF gene that can be used to support the growth of a production strain that has an inactivated pyrF gene (pyrF minus). In some embodiments, prior to transformation of the phagemid carrying the pyrF selection sequence into the pyrF minus production strain, the production strain is cultured in a medium supplemented with uracil. After transformation with the phagemid with the pyrF selectable sequence, the production strain is cultured in a medium without the uracil supplement. In some embodiments, the pyrF gene comprises a sequence having at least 80%, at least 85%, at least 90%, at least 95%, at least 96%, at least 98%, at least 99%, or at least 99.5% sequence identity to the sequence set forth in SEQ ID NO: 34. In some embodiments, the pyrF gene comprises the sequence of SEQ ID NO: 34. Alternatively or additionally, in some embodiments, auxotrophy selectable sequences can be selected from genes making up for a production strain's deficiency in anabolism of one or more of adenine, cytosine, guanine, thymine, alanine, arginine, asparagine, aspartate, cysteine, glutamine, glutamate, glycine, histidine, isoleucine, leucine, lysine, methionine, phenylalanine, proline, serine, threonine, tryptophan, tyrosine, valine, biotin, uridine-5′-monophosphate, pantothenate, xanthine, spermidine, para aminobenzoate, lipoate, nicotinamide riboside, nicotinamide mononucleotide, D-glucosamine, thiamine, shikimate, aminoethyl-phosphonate, beta alanine, s-methyl-methionine, ornithine, indole, indole acetic acid, L-threonine, L-threonine O-3-phosphate, nicotinate, ribosylnicotinamide, agmatine, etc., and/or from genes for the synthesis of other essential compounds.

In some embodiments, a production strain includes or achieves variable expression of phage genes (e.g., phage genes that are expressed at levels that are different from the levels of expression of the native gene, i.e., in its native genome). Variable expression includes overexpression and decreased expression compared to the level of expression of a phage gene in its native genome. In some embodiments, the ratio of gene expression between various phage genes is varied (e.g., varied from the native ratio of expression of native phage genes in their native genome).

Those skilled in the art will be familiar with various promoters that may be useful to vary the expression of a single phage gene or multiple phage genes. In some embodiments, promoters can be chosen that cause the overexpression of a phage gene as compared to that gene when it is expressed from its native promoter (e.g., when integrated and expressed from its native promoter or when expressed from its native promoter in its native bacteriophage genome). Conversely, in some embodiments, promoters can be chosen that cause reduced expression compared to that of the native gene (e.g., when integrated and expressed from its native promoter or when expressed from its native promoter in its native bacteriophage genome). Alternatively or additionally, with respect to either of the foregoing, in some embodiments, promoters can be chosen to achieve a particular timing, pattern, or responsiveness (e.g., to an environmental or applied trigger) of expression. In some embodiments, cssDNA production can be improved (e.g., optimized) for a given production stain and phagemid combination through the use of such promoters (e.g., heterologous promoters) and/or through integration of some or all relevant phage genes in the production strain genome. Particularly useful promoters in certain embodiments may include, for example, canonical T7 promoters, or mutant T7 promoters under the control of an inducible T7 polymerase, lacI promoters, lacIq promoters, araBAD promoters, tet promoters, temperature sensitive promoters, stress responsive promoters, quorum sensing promoters, light sensitive promoters or other inducible or repressible promoters.

In some embodiments, a production strain provided in accordance with the present disclosure can include genomically integrated phage protein coding sequences that are integrated at more than one locus within a production strain genome. Selection of loci within a production strain genome can influence expression level of the phage coding sequence and can be chosen to optimize the production strain performance. In some embodiments, phage protein coding sequences can be genomically integrated at least 2, 3, 4, 5, 6, 7, 8, 9, 10, or 11 distinct loci within the genome.

One of ordinary skill in the art will appreciate that in addition or as an alternative to manipulations in the control sequences controlling the expression of the phage proteins, in some embodiments, variants of naturally occurring phage proteins may be utilized. In some embodiments, a phage protein variant comprises an altered sequence with respect to its native naturally occurring sequence. In some embodiments, phage protein variants have altered expression, activity, and/or sequence as compared to a naturally occurring phage protein. Those skilled in the art will be aware of a variety of technologies for altering protein sequences (and/or sequences of nucleic acids that encode them), and furthermore will appreciate that, in some embodiments, activity of a protein that is known in the art can be used to generate the desired phage protein with the desired characteristics. Exemplary methods include, random mutagenesis, rational design, assisted lab evolution, directed evolution or other means. In some embodiments, modifications to a protein can be made and then tested to determine if their desired effect is achieved, for example by measuring cssDNA yield, quality and/or fidelity.

Alternatively or additionally, in some embodiments, selection sequences can be or include synthetic tRNA sequences that can (and, in some embodiments, may be necessary to) recognize a recoded codon that codes for an unnatural amino acid that is included in at least one essential gene in a production strain as described herein. In such embodiments, a production strain will not be able to grow without the presence of the unnatural amino acid and the synthetic tRNA sequence that recognizes the recoded codon for the unnatural amino acid.

For example, E. coli can be genomically recoded such that the UAG codon becomes the dedicated codon for the unnatural amino acid L-4,4′-biphenylalanine (bipA, also referred to as the bipA chemical) if the bipA aminoacyltRNA synthetase is expressed in the cell. Mandell et al., Biocontainment of genetically modified organisms by synthetic protein design. Nature 518, 55-60 (2015). The UAG codon can be inserted into three essential genes such that these genes cannot be translated if either the bipA chemical or the bipA tRNA is absent. For example, the UAG codon can be inserted into the essential genes adenylate kinase (adk.d6), tyrosyl-tRNA synthetase (tyrS.d8), and BipA-dependent aminoacyl-tRNA synthetase—for aminoacylation of BipA (BipARS.d6). Kunjapur et al., Synthetic auxotrophy remains stable after continuous evolution and in co-culture with mammalian cells. Sci Adv. 2021 Jul. 2; 7(27):eabf5851. In this scenario, the bipA IRNA can be expressed from the phagemid and bipA chemical can be added to the media creating a situation where only the E. coli cells harboring the phagemid can survive ensuring that virtually all E. coli cells in the population will contain the phagemid even though it has not been selected for with antibiotics. Furthermore, the E. coli cells will not be able to survive outside of the specific culture where the bipA chemical has been added to the medium since bipA is not available in the environment. This prevents the engineered production host from escaping the lab or fermentation facility environment. It also prevents the production host from contaminating fermentation vessels that may be used for other strains for example at a contract manufacturing organization (CMO), which is a concern considering the production host expresses phage particles which could theoretically infect other bacterial strains at a CMO.

In some embodiments, one or more phage proteins, either expressed from a helper plasmid, helper virus, or from the genome of the production strain, can additionally include a tag. The tag can be useful for identifying, quantifying, and/or separating the phage particles. Suitable tags include fluorescent tags, luminescent tags, chromophoric tags, and affinity tags. Particularly useful affinity tags include biotin, his, myc, flag, CBP, GST, HA, HBH, MBP, S, and V5 or other affinity tags to aid in purification of phage particles from production broth.

Among other things, the present disclosure provides certain methods of producing cssDNA. In certain embodiments, such methods include culturing a production strain that includes at least two phage protein coding sequences genomically integrated into the production strain genome in a media and introducing a phagemid into the production strain. Those skilled in the art will be familiar with a variety of technologies for introducing a phagemid into a production strain. Exemplary such technologies include, for example, conjugation, transduction, electroporation, chemical transformation, or by exploiting the native phage infection mechanism. Resulting phage particles can be collected and the cssDNA can be collected. Exemplary means of separation include centrifugation, gel electrophoresis, membrane separation, tangential flow filtration, liquid chromatography, etc. In some embodiments, means of separating cssDNA include the substantial separation of any dsDNA from the cssDNA. Those skilled in the art will be aware of a variety of technologies for quantifying cssDNA such as, for example, absorption spectra, fluorescent intercalating dye, UV detection, gel electrophoresis, microscopy, and the like. In embodiments where the phage particles additionally comprise a tag, that tag can be used to aid in the separation and/or quantification steps.

Additional methods of producing cssDNA are also provided, for example wherein a cssDNA production system is used. In some embodiments, a provided cssDNA production system uses a production strain with phage particle production sequences already included. Exemplary such methods include culturing the cssDNA system in media and optionally inducing the production of one or more of the phage genes, collecting the phage particles and separating the phage particles. In some embodiments, the collection and separation steps can be simultaneous, particularly when an affinity tag is used allowing for the separation of the phage particle coat away from the cssDNA in a single step.

### BRIEF DESCRIPTION OF THE DRAWINGS

FIGS. 1A-B shows a schematic representation of M13 phage particle depicting the organization of coat proteins around the ssDNA genome (FIG. 1A). FIG. 1B shows a map of the M13 phage genome identifying all genes, the intergenic region, promoters (upper case P) and terminators (upper case T). Coding regions for each M13 protein are labeled “p #”.

FIG. 2 shows a phagemid comprising an F1 origin of replication; a selectable sequence; a user-defined sequence; and an optional plasmid origin of replication.

FIG. 3 shows a production strain with at least two phage coding sequences genomically integrated and a phagemid that includes a phage origin of replication, packaging signal, a selection sequence and a designed sequence.

FIG. 4 shows a map of plasmid M13KO7 that contains bacteriophage M13 genes I-XI, the p15A origin of replication, the M13 origin of replication and packaging signal and the kanamycin resistance marker.

FIG. 5 shows a plasmid expressing the lambda red recombineering system including beta, exo and gamma from an arabinose inducible promoter (catalog number CAS9BAC1P, Sigma Aldrich, Burlington, Massachusetts).

FIG. 6 shows a schematic of the accumulation of additional cssDNA template within the E. coli cell resulting in increased production of packaged cssDNA (Lee et al., Optimizing protein V untranslated region sequence in M13 phage for increased production of single-stranded DNA for origami. Nucleic Acids Res. 2021 Jun. 21; 49(11):6596-6603, which is herein incorporated by reference in its entirety).

FIG. 7 shows PCR primers designed to amplify a first transcriptional unit such that the 5′ UTR (untranslated region) of gene V is changed from the wild-type TCACA to GAGGT (FIG. 7, panel A). FIG. 7, panel B shows a schematic of PCR primers designed to amplify the rest of Transcription Unit 1 through fusion PCR, such that the mutated 5′ UTR of gene V is incorporated into the Transcription Unit 1 creating a 2117 bp variation of Transcription unit 1 including the altered gene V 5′ UTR sequence.

FIGS. 8A-8B show a phagemid map including a pUC origin of replication (FIG. 8A), and a portion of the pUC ori sequence and primers used for generating inc1 and inc2 mutations.

FIG. 9 provides results demonstrating the effect of phagemid origin of replication on cssDNA production.

FIG. 10 provides the results of larger scale culture experiments showing that inc1 and inc2 mutations increase cssDNA yield.

FIG. 11 provides the results of qPCR experiments to analyze relative cssDNA yield using helper plasmids with different origins of replication.

FIG. 12 shows a comparison terminal OD for a variety of potential bacterial production strains.

FIG. 13A shows the verification of cssDNA production in the unengineered BW25113 parent strain. FIG. 13B shows results demonstrating induction of pLac:T7 polymerase expression in an engineered bacterial strain bac058 containing the integrated pLac:T7 construct and an emGFP reporter.

FIG. 14A shows the results of a screen of a small combinatorial library of integrated M13 TU1 and TU2 driven by different strength promoters.

FIG. 14B shows the results comparing select engineered strains to their non-engineered parent strain.

FIG. 15A shows a comparison of best engineered production host (bac105) to its parent strain and to the traditional production strain DH5α using the traditional helper plasmid system.

FIG. 15B shows the results for production of various cssDNA sequences from engineered host strains.

FIG. 15C shows a comparison of cssDNA production by the engineered bac105 production strain with its parent strain bac016 and the traditional production host bac001 when transformed with the RFP/ampicillin phagemid cdsDNA111, and bac105 cssDNA production when transformed with the RFP/pyrF phagemid cdsDNA117.

### DETAILED DESCRIPTION

The production of ssDNA of any significant length is complicated in part because during chemical synthesis as the DNA molecule increases in length the success of adding the next base to the polymer decreases. Similarly, production of longer ssDNA polymers using PCR based techniques or through plasmid leads to the need to remove any regions of dsDNA and therefore, the procedures become complicated and lead to metabolic waste. For example, in some instances, double-stranded DNA is made using typical plasmid amplification schemes at a metabolic cost to the host organism and then subsequently the undesired strand is enzymatically removed. This post assembly modification method leads to a loss of cssDNA product and a wide range of ssDNA lengths given the inaccuracy of the subsequent enzymatic removal step. It is also metabolically wasteful to synthesize large amounts of dsDNA only to degrade one the two strands thus wasting half of the deoxyribonucleotide triphosphate molecules (dNTPs) that had been synthesized and polymerized.

Bacteriophages have been used to make ssDNA and cssDNA, and production of these products has been reported. Bush et al., Synthesis of DNA Origami Scaffolds: Current and Emerging Strategies, Molecules 2020, 25, 3386. However, engineered bacteriophages that contain DNA that is not from the native bacteriophages, for example fluorescent protein tags, have significantly reduced cssDNA yield compared to production of the native bacteriophage. This leaves opportunities for improving the efficiency of such systems to make ssDNA and cssDNA.

ssDNA and cssDNA are desirable for several known applications and it is predicted that as technology related to the fields of DNA data storage and DNA nanotechnology (i.e., DNA origami) grows, more applications will be found. For example, dsDNA from salmon roe has been shown to be an effective transparent sunscreen (Gasperini, et al., Non-ionising UV light increases the optical density of hygroscopic self assembled DNA crystal films 2017 Nature Scientific Reports 7: 6631). However, its commercial usage is likely impractical given the cost associated with its manufacture. Synthetic biology engineering combined with efficient biotechnological production methods can be used to produce such DNA in a cost-effective manner.

### Definitions

Unless otherwise noted, technical terms are used according to conventional usage. Definitions of common terms in molecular biology can be found in e.g., Benjamin Lewin, Genes XII, Jones & Bartlett Learning; 12th edition (Mar. 16, 2017) and other similar references. As used herein, the singular forms “a,” “an,” and “the,” refer to both the singular as well as plural, unless the context clearly indicates otherwise. For example, the term “a peptide” includes single or plural antigens and can be considered equivalent to the phrase “at least one peptide.” As used herein, the term “comprises” means “includes.” Thus, “comprising a protein” means “including a protein” without excluding other elements. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. For instance, in some embodiments control sequences are used in engineered nucleic acid sequences to express a coding sequence, the control sequences used are not the same as the control sequences that are found in the natural gene that expresses the protein in a microorganism. The use of control sequences that are distinct from those found in nature can be referred to as non-endogenous as compared to the coding sequence. In such instances the activity of the natural gene expression cassette can be compared to the engineered version of the gene made with the non-endogenous control sequences. In such comparisons the non-endogenous control sequences can be found to be active or inactive as compared to the natural gene expression cassette when both expression cassettes are expressed under substantially the same experimental conditions.

“Control sequences” refer to nucleic acid sequences that regulate the expression of a nucleic acid sequence to which the control sequence is operatively linked. Expression control sequences are operatively linked to a nucleic acid sequence when the expression control sequences control and regulate the transcription and, as appropriate, translation of the nucleic acid sequence. Thus, the term “expression control sequences” can refer to elements such as promoters, enhancers, transcription terminators, ribosome binding sites and start codons (ATG) in front of a protein-encoding gene, splicing signal for introns, maintenance of the correct reading frame of that gene to permit proper translation of mRNA, stop codons, etc. The term “control sequences” is intended to include, at a minimum, components whose presence can influence expression, and can also include additional components whose presence is advantageous, for example, leader sequences and fusion partner sequences. Expression control sequences can include a promoter.

The term “designed sequences” as used herein refers to nucleic acid sequences that are engineered to be included in a template cssDNA, template ssDNA, phagemid, or host cell genome. In some embodiments, designed sequences are intended to be produced by production strain described herein upon introduction into the production stain. Designed sequences can include genomic editing sequences, structural sequences for example for use in DNA origami, or any other nucleic acid sequence desired.

In general, the term “engineered” or “synthetic” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that it's genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity. Other examples of the use of the term “synthetic” include synthetic nucleases. Synthetic nucleases are nucleases that additionally include a domain that directly, or indirectly, associates with a co-location sequence.

A “promoter” as used herein refers to a minimal sequence sufficient to direct transcription. A promoter is also part of a group of nucleic acid sequences commonly referred to as a control sequence. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used.

“cssDNA” as used herein refers to circular single-stranded deoxyribonucleic acid. cssDNA, as described herein, can be packaged into a phage or phage particle.

A “functionally disabled gene/protein/polypeptide” refers to one that does not exert its natural biological function or activity. Thus, for example, a functionally inactivated gene is a gene that cannot be transcribed and/or translated into a protein or polypeptide that exerts the biological function or activity of the protein or polypeptide that is encoded by the native gene. Inactivation of genes may be accomplished in a number of ways, including without limitation, by deletion, substitution, insertion, mutation, and introduction of stop codons, frame shifts, truncations and the like. A functionally disabled protein can be also described as having decreased or no activity.

“Genomic editing sequences” as used herein are a specific type of designed sequence. Genomic editing sequences are intended to cause alterations in the host cell genome. In some instances, a genomic editing sequence can include one or more regions that are homologous to the host cell genome. In some instances, a genomic editing sequence can contain an entire gene expression cassette, for example, a promoter, open reading frame and a terminator. In other embodiments, a genomic editing sequence can include a gene or a fraction of a gene such as intron, exon, or the like. A genomic editing sequence in a phagemid can be designed to alter the genome of a host cell such that the performance of the endogenous nucleic acid sequence in the host cell is changed. In some instances, genomic editing sequences can be designed to delete, down-regulate or diminish the activity of an endogenous genomic product. In yet other examples, genomic editing sequences can be designed to up-regulate or increase expression of an endogenous genomic product. In yet other examples, genomic editing sequences can be designed to repair an undesirable sequence, such a premature stop codon, that is found in the host cells endogenous genome. Genomic editing sequences can include, for example, cssDNA molecules that upon incorporation into the genome of a host cell produce siRNA, ribozymes, antisense sequences, RNAi, genes, corrective sequences for various genetic diseases, or beneficial genes or other sequences.

“Introduced” as referred to herein means the movement of a nucleic acid sequence or protein into a cell through, e.g., infection of a phage (transduction), conjugation, or transformation with a molecular biology technique, such as electroporation or heat shock of chemically competent cells.

“Non-endogenous” as used herein refers to sequences, proteins, etc. that are not endogenous to a particular host cell (e.g., are not naturally occurring in their relevant environment). For example, in some embodiments, a non-endogenous sequence is introduced into a production strain genome, and the non-endogenous sequence is not found naturally in the production strain genome prior to genetic engineering.

“Nucleic acid”, “polynucleotide”, and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid). In general and unless otherwise specified, an analogue of a particular nucleotide has the same base-pairing specificity; i.e., an analogue of adenine will base-pair with thymine. When double-stranded DNA is described, the DNA can be described according to the conformation adopted by the helical DNA, as either A-DNA, B-DNA, or Z-DNA. The B-DNA described by James Watson and Francis Crick is believed to predominate in cells, and extends about 34 Å per 10 bp of sequence; A-DNA extends about 23 Å per 10 bp of sequence, and Z-DNA extends about 38 Å per 10 bp of sequence.

In some cases, nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some embodiments the set of characters is (A, C, G, T, U) for adenosine, cytidine, guanosine, thymidine, and uridine respectively.

Nucleotide refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

“Oligonucleotide” or a “polynucleotide” refer to synthetic or isolated nucleic acid polymers including a plurality of nucleotide subunits.

A “phagemid” as used herein includes a bacteriophage origin of replication, a selection sequence, a designed sequence, and a packaging signal. In some embodiments a phagemid can also include a plasmid origin of replication and/or an antibiotic resistance marker. The sequence of the phagemid will not include phage protein coding sequences so that a cell, upon infection by the phagemid, will not produce additional phage particles, unless the cell additionally includes coding sequences for the necessary phage proteins.

A “phage particle” as used herein refers to a cssDNA sequence that is coated with phage proteins, but yet does not include the coding sequences necessary for producing phage proteins.

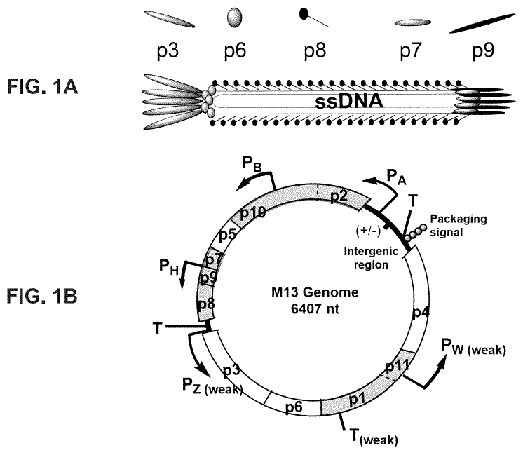

“Phage proteins” as used herein refer to the proteins that are encoded by a native bacteriophage that are needed for phage replication in a host. See e.g., FIGS. 1A and 1B, which illustrate the M13 phage proteins and M13 genomic structure, respectively. In some embodiments, phage proteins include phage proteins that have nucleic acid sequence modifications as compared to the native sequence. Such modifications include for instance altering the nucleic acid sequence encoding the phage protein to optimize based on codon usage of a particular production host. In some embodiments, such modifications result in altered activity of the phage proteins. Nucleic acid sequences encoding phage proteins can also be altered to produce phage proteins that have alternative amino acids as compared to the native phage proteins. In some instances the phage protein can include at least 1, 2, 3, 4, 5, 10, 15, 20, 25, or 30 amino acids that are distinct from the native phage sequence. Additional modifications that can be included in phage proteins include the addition of amino acids for example tags such as fluorescent markers. The phage proteins described herein, for instance native phage proteins or modified phage proteins, will continue to function in the production strain. For example, a phage protein comprising a tag may have looser binding to other phage proteins when a tag is included, but yet it is still considered a phage protein. Phage proteins that are specifically described herein include the M13 phage proteins and nucleic acid sequences provided in the sequence listing. The proteins are referred to as p1, p2, p3, p4, p5, p6, p7, p8, p9, p10 and p11 and the genes are referred to using the respective roman numerals (I, II, III, IV, V, VI, VII, VIII, IX, X and XI). If the phage protein is modified the modified phage protein may be characterized through comparison to the native phage protein.

The terms “polypeptide”, “peptide” and “protein” are used interchangeably to refer to a polymer of amino acid residues. The term also applies to amino acid polymers in which one or more amino acids are chemical analogues or modified derivatives of corresponding naturally-occurring amino acids.

A “production strain” as used herein refers to a bacterial cell used for production of ssDNA (e.g., cssDNA). In some embodiments, a production strain comprises at least two phage proteins integrated into the genome of the production strain. In some embodiments, the at least two phage proteins can be differentially expressed as compared to the level of expression that would result if the endogenous control sequences from the native bacteriophage were used. For example, one or more of the at least two phage protein sequences can be under the control of an inducible promoter. In some embodiments the production strain is a strain of E. coli, for example the non-toxic E. coli described in U.S. Pat. No. 8,303,964 (which is herein incorporated by reference), which is additionally engineered to reduce the presence of the 3-deoxy-d-manno-oct-2-ulosonic acid (Kdo) component of the cell capsule.

“Selection sequences” or “selectable sequences” as referred to herein are sequences of nucleic acids or amino acids that upon inclusion in the phagemid sequence allow the phagemid to be retained within the production strain population. Selectable sequences include for example antibiotic resistance sequences, engineered tRNA sequences, auxotrophic markers, antitoxin genes, or sequences that alter transcription or translation. Examples of selectable sequences include, without limitation, genes encoding proteins that increase or decrease either resistance or sensitivity to antibiotics (e.g., ampicillin resistance genes, kanamycin resistance genes, neomycin resistance genes, tetracycline resistance genes, chloramphenicol resistance genes, and spectinomycin resistance genes) or other compounds. Additional examples of selectable sequences include, without limitation, genes encoding proteins that enable the cell to grow in media deficient in an otherwise essential nutrient such as uracil, leucine, adenine, histidine, arginine, lysine, tryptophan, methionine or other essential metabolites. In some examples, the production host is engineered to diminish or eliminate the production of an essential nutrient.

“Template cssDNA” as used herein refers to circular single stranded DNA used in the production of cssDNA. In some embodiments, template cssDNA includes a packaging sequence, designed sequence, and a phage ori, which upon introduction into a bacterial cell, such as an E. coli cell, is replicated into a replicative form using the bacterial cell proteins and nucleic acid sequences, and then is packaged into phage particles.

As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to one another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature.

Making cssDNA

The present disclosure provides, among other things, methods of making cssDNA. One of ordinary skill in the art, upon reading the disclosure, will appreciate that the designed sequence included in the phagemid can be any length necessary to accomplish the desired function of the designed sequences. For example, if the desired sequence is a three-dimensional DNA structure (i.e., DNA origami) the length will be chosen to accommodate the size of the desired end product. Similarly, if the designed sequence is for eventual use in a cell or gene therapy application, such as CAR-T cell therapy, the sequence can be the length of a gene that is targeted for insertion plus the length of the homology arms necessary to target the gene to a specific locus. Particular uses of the cssDNA include use in combination with genomic editing techniques such as homologous recombination and CRISPR/Cas related techniques. Given the variety of uses of the cssDNA it is expected that the designed sequences will range from about 10 bp to about 10,000 bp, from about 100 bp to about 10,000 bp, from about 200 bp to about 9000 bp, from about 300 bp to about 9000 bp, from about 500 bp to about 9000 bp, from about 500 bp to about 8000 bp, from about 1000 bp to about 10,000 bp, from about 5,000 bp to about 15,000 bp, and from about 10,000 bp to about 30,000 bp, cssDNA produced by methods described herein, in some embodiments are consistent, for example, greater than 95%, 96%, 97%, 98%, 99%, 99.5% or 99.9% of the molecules produced will have no mutations relative to the template. In some embodiments, molecules can also be greater than 10,000, 20,000 or 30,000 bp in length.

In some embodiments, cssDNA production occurs when a dsDNA phagemid encoding a phage origin of replication and a packaging signal, but lacking phage genes is replicated as cssDNA by the phage proteins expressed in trans in the production strain host and then packaged into the phage particle by the phage proteins. A phage particle, when produced as described above, does not contain any coding sequences for any phage proteins, making it incapable of replicating itself if it were to infect another bacterial cell. This is different from a wild type phage which encodes the genes necessary for its own replication in its genome and can replicate itself and produce new, self-replicating phage particles capable of infecting other bacterial cells and creating still more self-replicating phage. The phagemids produced herein can include a phage origin of replication and a plasmid origin of replication. However, in some instances, the phagemid includes only the phage origin of replication and lacks a plasmid origin of replication.

In some embodiments, phagemids described herein include one or more selectable sequences. Selectable sequences function to tie the phagemid to the production strain such that the production strain will not flourish without the presence of the phagemid encoding the selectable sequence. Selectable sequences include sequences that encode antibiotic resistance proteins, auxotrophy amino acids; tRNAs that compensate for deficiencies that are engineered into the production strain; RNA sequences that alter transcription or translation; or antitoxin amino acids (wherein the production strain produces a toxin). One of ordinary skill in the art will appreciate that there are many specific examples of such amino acid sequences and nucleic acid sequences that can be used to create a relationship between the phagemid and the production strain such that the production strain cannot continue to fully function without the phagemid. In preferred embodiments, sequences encoding genes or proteins that can have a deleterious effect on the end product that the cssDNA will be used for are avoided. For example, in instances where the cssDNA is being used for gene therapy or microbial engineering of microbes that will be released into the environment, antibiotic resistance sequences and toxin expressing sequences are not desirable in the cssDNA.

One of ordinary skill in the art will appreciate that there are several methods of making a phagemid that does not include a plasmid dsDNA origin of replication. This can be useful if it is undesirable for the plasmid origin of replication to exist in the final cssDNA that is produced. For example, the desired phagemid sequence can be cloned into a plasmid with additional control sequences such as a T7 promoter sequence, a designed sequence, a selection sequence and a packaging signal sequence. An in vitro transcription reaction can be conducted and then the resulting RNA can be reverse transcribed into the desired ssDNA sequence. The resulting ssDNA can be circularized into cssDNA using any method known in the art, for example a splint (short DNA that has regions of complementarity to both ends of the ssDNA) can be annealed and the reaction can be subjected to a ligation reaction to anneal the ends to form cssDNA. Iyer et al., Efficient Homology-directed Repair with Circular ssDNA Donors, CRISPR J, October; 5(5):685-701, 2022. ssDNA ligases can also be used to convert the linear ssDNA into cssDNA.

Alternatively, the desired components of a ssDNA can be engineered using a combination of synthetic DNA synthesis and standard cloning and PCR techniques. The sequence can be created in a double stranded form and then lambda exonuclease can be used to selectively remove a strand of the DNA.

Additional techniques also include the use of streptavidin coated beads and PCR primers conjugated to biotin where one primer is biotinylated while the other is not. After PCR is complete the one strand is biotinylated and the other is not. Exonuclease is applied to degrade the non-biotinylated strand. The resulting biotinylated ssDNA product is allowed to bind to the streptavidin coated beads and the ssDNA is recovered by physically separating the beads from the solution and then eluting the DNA from the beads by resuspending them in an elution buffer. Avci-Adali M, Paul A, Wilhelm N, Ziemer G, Wendel H P. Upgrading SELEX technology by using lambda exonuclease digestion for single-stranded DNA generation. Molecules. 2009 Dec. 24; 15(1):1-11.

ssDNA that will eventually be packaged into the phage particle can also be cloned into the host cell genome as part of an expression cassette that allows for the direct production of an RNA transcript having the desired phagemid components. Transcription can be induced at a desired time point and upon transcription the resulting transcript can be reverse transcribed allowing the packaging signal to be accessible to the rest of the phage genes necessary for packaging. Phage genes encoding coat proteins can be either genomically integrated or supplied through a helper virus or a helper plasmid. One of ordinary skill in the art will appreciate that it may be necessary to additionally express the desired reverse transcriptase.

The phagemid can also be produced without a plasmid origin of replication using any standard in vitro dsDNA cloning methods including restriction/ligation, isothermal assembly, Golden Gate Assembly or any other method used to assemble DNA fragments. Once produced in vitro, circular dsDNA can then be transformed into any bacterial strain that expresses the necessary phage machinery to replicate and package cssDNA from the dsDNA template that includes the phage origin of replication and the phage packaging signal.

In instances where the phagemid includes a phage origin of replication and a plasmid origin of replication, standard plasmid cloning techniques can be used to create the desired template sequence for making the cssDNA product. The inclusion of the plasmid origin of replication is convenient for many genetic engineering techniques, but including the plasmid origin of replication also raises the possibility that the plasmid origin of replication sequence will be included in the cssDNA product, which in some instances, may not be desirable.

In some embodiments, the phagemid is a high copy number phagemid. In some embodiments, the phagemid copy number in production strain cells grown to late log phase is at least 1,000, optionally wherein the phagemid copy number in production strain cells grown to late log phase is at least 2,000, at least 4,000, at least 7,000, at least 8,000, or at least 15,000. As shown in Example 6, in some embodiments, a phagemid comprising one or more mutations that increase copy number increases cssDNA yield. In some embodiments, the phagemid comprises an inc1 mutation. In some embodiments, the phagemid comprises an inc2 mutation. In some embodiments, the phagemid comprises an inc1 mutation and an inc2 mutation. In some embodiments, the phagemid comprises a pST19, pDHA29, pDHA30, pDHK29, pDHK30, or runaway R1 origin of replication or derivative thereof.

In some embodiments, the phagemid comprises a pUC origin of replication (ori). In some embodiments, the phagemid ori comprises a sequence having at least any of 95%, 96%, 97%, 98%, 99%, or 100% sequence identity to SEQ ID NO: 27. In some embodiments, the phagemid comprises an inc1 mutated ori derived from a pUC ori. In some embodiments, the inc1 mutated ori comprises a sequence having at least any of 95%, 96%, 97%, 98%, 99%, or 100% sequence identity to SEQ ID NO: 28. In some embodiments, the phagemid comprises an inc1 mutated ori derived from a pUC ori. In some embodiments, the inc1 mutated ori comprises a sequence having at least any of 95%, 96%, 97%, 98%, 99%, or 100% sequence identity to SEQ ID NO: 31. In some embodiments, the inc1 mutation is a C59T mutation relative to a standard pUC origin, wherein the nucleotide numbering is based on the sequence of the standard pUC origin shown in SEQ ID NO: 27. In some embodiments, the phagemid comprises an inc2 mutated ori derived from a pUC ori. In some embodiments, the inc2 mutation is a C92T mutation relative to a standard pUC origin, wherein the nucleotide numbering is based on the sequence of the standard pUC origin shown in SEQ ID NO: 27. In some embodiments, the inc2 mutated ori comprises a sequence having at least any of 95%, 96%, 97%, 98%, 99%, or 100% sequence identity to SEQ ID NO: 29. In some embodiments, the inc2 mutated ori comprises a sequence having at least any of 95%, 96%, 97%, 98%, 99%, or 100% sequence identity to SEQ ID NO: 32. In some embodiments, the phagemid comprises an inc1 and inc2 mutated ori derived from a pUC ori. In some embodiments, the inc1 &2 ori (also referred to as inc1/inc2 or inc1/2) comprises a C59T mutation and a C92T mutation relative to a standard pUC origin, wherein the nucleotide numbering is based on the sequence of the standard pUC origin shown in SEQ ID NO: 27. In some embodiments, the inc1 and inc2 mutated ori (inc1 &2 ori) comprises a sequence having at least any of 95%, 96%, 97%, 98%, 99%, or 100% sequence identity to SEQ ID NO: 30. In some embodiments, the inc1 and inc2 mutated ori (inc1 &2 ori) comprises a sequence having at least any of 95%, 96%, 97%, 98%, 99%, or 100% sequence identity to SEQ ID NO: 33. In some embodiments, the phagemid comprises an inc3 mutation. In some embodiments, the phagemid comprises an inc5 mutation. In some embodiments, the phagemid copy number in production strain cells grown to late log phase is at least 1,000. In some embodiments, the phagemid copy number in production strain cells grown to late log phase is at least 2,000. In some embodiments, the phagemid copy number in production strain cells grown to late log phase is at least 4,000. In some embodiments, culturing a cssDNA production strain or system comprising a phagemid containing an inc1&2 ori results in at least 10-fold higher yield compared to culturing a cssDNA production strain or system comprising a phagemid containing a wild-type pUC ori. In some embodiments, phagemid, once produced, can be introduced into a production strain using any method known in the art, including, for example, electroporation or through the use of chemically competent cells such as those made with divalent ions such as calcium chloride, magnesium chloride or rubidium chloride solutions.

### Making Production Strains

The present disclosure exemplifies production strains and methods of engineering the same. Described production strains are useful, among other things, in producing cssDNA. Those of ordinary skill in the art will be familiar with a variety of technologies that may be useful to generate such production strains. Production strains described herein include strains of bacteria that can produce cssDNA phage particles after transformation with a phagemid, or nucleic acid sequence including the phage packaging signal, a designed sequence and a phage origin of replication. The bacteria can naturally include the intracellular machinery necessary to produce cssDNA from the template dsDNA, ssDNA or cssDNA, or the bacteria can be engineered to include such machinery. Machinery as used herein refers to the proteins necessary for the creation of cssDNA, rolling circle replication (sometimes also referred to herein as ssDNA production), or ssDNA packaging (sometimes also referred to as phage particle packaging). Production strains as described herein can be natural targets for infection by the phage containing the template cssDNA or they can simply be engineered to have the correct supporting protein production to produce phage particles upon introduction of the template cssDNA or dsDNA using a standard transformation technique. Exemplary bacteria that can be used to make a production strain include E. coli and other Gram negative bacterial species that are capable of replicating cssDNA phage genomes of cssDNA phages, e.g., of the realm of Monodnaviria that includes phages Ff, Fd, F1 and M13 among others.

In some embodiments, the production strain includes a copy of the native genes from a bacteriophage, such as an M13 bacteriophage, genomically integrated into one or more loci. For example, the genes encoding the phage proteins can be PCR amplified from the M13KO7 helper phage genome and integrated into the genome of a production strain as described herein. These production strains will be able to produce cssDNA upon introduction of a template dsDNA, cssDNA or ssDNA, as described herein. Production strains that comprise at least one copy, or at least two copies, of the genes encoding the phage proteins can also include additional copies of one or more of the phage protein genes. For example, the phage genes I, II, III, IV, V, VI, VII, VIII, IX, X and XI can be genomically integrated using their endogenous control sequences as described herein, and an additional copy of any one of the genes I, II, III, IV, V, VI, VII, VIII, IX, X, and XI can be genomically integrated or expressed from a helper plasmid using either the genes' native control sequence or an heterologous control sequence such as an inducible promoter.

In some embodiments, provided herein is a circular single-stranded DNA (cssDNA) production system, comprising: a production strain; and a phagemid, wherein the phagemid comprises a packaging signal, a designed sequence, and at least one selectable sequence,
wherein the phagemid comprises a pUC origin of replication or a derivative thereof, and wherein the phagemid comprises an inc1 mutation and/or an inc2 mutation. In some embodiments, the phagemid comprises an inc1 mutation and an inc2 mutation. In some embodiments, the production strain comprises two or more phage genes selected from the group consisting of genes I, II, III, IV, V, VI, VII, VIII, IX, X and XI genomically integrated into the production strain cells. In some embodiments, the phage genes are expressed from a helper plasmid (additionally or alternatively to integrated phage genes). In some embodiments, the production strain comprises a helper plasmid derived from helper phage M13KO7 by removing the F1 origin and the packaging signal. In some embodiments, the helper plasmid copy number in production strain cells grown to late log phase is at least 1,000. In some embodiments, the helper plasmid copy number in production strain cells grown to late log phase is at least 2,000, at least 4,000, at least 7,000, at least 8,000, or at least 15,000. In some embodiments, the helper plasmid comprises a pUC origin of replication.

In some embodiments, the helper plasmid comprises an inc1 mutation. In some embodiments, the helper plasmid comprises an inc2 mutation. In some embodiments, the helper plasmid comprises an inc1 mutation and an inc2 mutation. In some embodiments, the helper plasmid comprises a pST19, pDHA29, pDHA30, pDHK29, pDHK30, or runaway R1 origin of replication or derivative thereof.

In some embodiments, the helper plasmid comprises a pUC origin of replication (ori). In some embodiments, the helper plasmid ori comprises a sequence having at least any of 95%, 96%, 97%, 98%, 99%, or 100% sequence identity to SEQ ID NO: 27. In some embodiments, the helper plasmid comprises an inc1 mutated ori derived from a pUC ori. In some embodiments, the inc1 ori comprises a C59T mutation relative to a standard pUC origin, wherein the nucleotide numbering is based on the sequence of the standard pUC origin shown in SEQ ID NO: 27. In some embodiments, the inc1 mutated ori comprises a sequence having at least any of 95%, 96%, 97%, 98%, 99%, or 100% sequence identity to SEQ ID NO: 28. In some embodiments, the inc1 mutated ori comprises a sequence having at least any of 95%, 96%, 97%, 98%, 99%, or 100% sequence identity to SEQ ID NO: 31. In some embodiments, the helper plasmid comprises an inc2 mutated ori derived from a pUC ori. In some embodiments, the inc2 ori comprises a C92T mutation relative to a standard pUC origin, wherein the nucleotide numbering is based on the sequence of the standard pUC origin shown in SEQ ID NO: 27. In some embodiments, the inc2 mutated ori comprises a sequence having at least any of 95%, 96%, 97%, 98%, 99%, or 100% sequence identity to SEQ ID NO: 29. In some embodiments, the inc2 mutated ori comprises a sequence having at least any of 95%, 96%, 97%, 98%, 99%, or 100% sequence identity to SEQ ID NO: 32. In some embodiments, the helper plasmid comprises an inc1 and inc2 mutated ori derived from a pUC ori. In some embodiments, the inc1 &2 ori (also referred to as inc1/inc2 or inc1/2) comprises a C59T mutation and a C92T mutation relative to a standard pUC origin, wherein the nucleotide numbering is based on the sequence of the standard pUC origin shown in SEQ ID NO: 27. In some embodiments, the inc1 and inc2 mutated ori (inc1 &2 ori) comprises a sequence having at least any of 95%, 96%, 97%, 98%, 99%, or 100% sequence identity to SEQ ID NO: 30. In some embodiments, the inc1 and inc2 mutated ori (inc1 &2 ori) comprises a sequence having at least any of 95%, 96%, 97%, 98%, 99%, or 100% sequence identity to SEQ ID NO: 33. In some embodiments, the helper plasmid comprises an inc3 mutation. In some embodiments, the helper plasmid comprises an inc5 mutation. In some embodiments, the helper plasmid copy number in production strain cells grown to late log phase is at least 1,000. In some embodiments, the helper plasmid copy number in production strain cells grown to late log phase is at least 2,000. In some embodiments, the helper plasmid copy number in production strain cells grown to late log phase is at least 4,000.

In some embodiments, production strains described herein can also contain truncated versions of gene III that decrease the production strain's resistance to subsequent infection by phage particles. Truncations can include deletions of amino acid residues 21-273, deletions of amino acid residues 93-121, deletions of amino acid residues 93-141, or the insertion of stop codons within the p3 gene at codons 20, 25, 28, 30, or the like. See U.S. Pat. No. 8,227,242, which is herein incorporated by reference in its entirety.

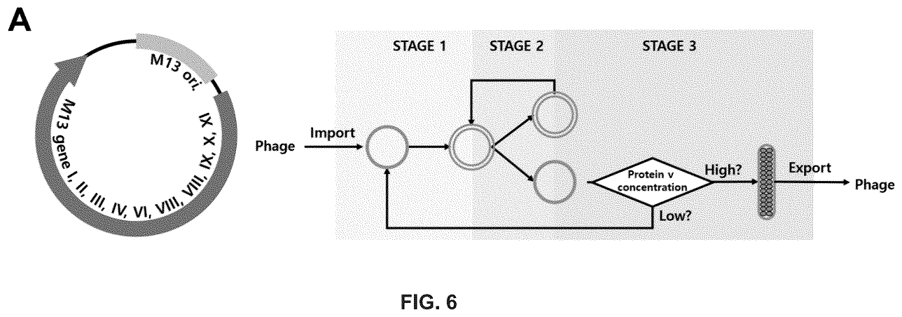

In some production strains, expression of the phage protein p5 is altered (e.g., decreased) to increase cssDNA production. Decreasing the activity of the p5 protein production can be accomplished using any method known in the art, including those already described. P5 activity can also be altered through a mutation in the untranslated region of the p5 gene as described in Lee et al., Optimizing protein V untranslated region sequence in M13 phage for increased production of single-stranded DNA for origami. Nucleic Acids Research, Volume 49, Issue 11, 21 Jun. 2021 (see e.g., FIG. 6).

Overexpression of a gene can be accomplished using any method known in the art. Generally, overexpression refers to an increase in the amount of protein produced as compared to the amount that the protein is usually expressed when it is expressed in its native genomic environment. For that reason, overexpression is sometimes described in relation to a second protein that is expressed. Particular examples of how overexpression can be accomplished include inserting multiple copies of a gene or switching the promoter or other control sequence to increase expression. The production strains described herein can contain a copy of the M13 genome including the M13 control sequences. For the purposes of clarity, the term, overexpression will be used to refer to the production of an M13 protein at a greater amount than would have been expressed if it was under the control of its native control sequences. Stated another way, if two copies of the M13 genome are included in a production strain, all of the phage proteins would be considered overexpressed, however, if only a single copy of the M13 genome is included with its native control sequences, then none of the phage proteins would be considered overexpressed.

Production strains described herein can be also described by the ratio of one protein to another. The ratio of the production of the individual phage proteins to each other can increase the efficiency of the production of the cssDNA product. For example, the expression of p2:p5 in a 1:1, 2:1, 3:1, or 10:1 ratio during the stationary phase or log phase of growth can be beneficial to phage particle production.

Overexpression of p2 and p10 through the use of an additional copy of the native genes, the coding sequences being placed under the control of a constitutive promoter, the coding sequences being placed under control of an inducible promoter, or some combination thereof, is believed to increase phage particle production. See, Behler, et al., Phage-free production of artificial ssDNA with Escherichia coli, Biotechnol Bioeng. 2022 October; 119(10):2878-2889.

p8 protein makes up the majority of the phage coat based upon its overall amino acid contribution. In fact, in a wild type M13 phage capsid, there are 2700 copies of the p8 protein. In engineered phage capsids containing larger than wild type 7.2 kb genome, the capsid would incorporate even more p8 protein because the capsid length is proportional to the size of the packaged DNA. Therefore, it is particularly useful to overexpress the p8 protein when the designed sequences become longer. Overexpression of p8 through the use of an additional copy of the native genes, the coding sequences being placed under the control of a constitutive promoter, the coding sequences being placed under control of an inducible promoter, or some combination thereof, is believed to increase phage particle production. In particular embodiments, production strains include overexpressed p8 protein and designed sequences at least 2 kb in length. In yet other embodiments the production strains include overexpressed p8 protein and designed sequences that are at least 3 kb, 4 kb, 5 kb, 6 kb, 7 kb, 8 kb, 9 kb, 10 kb, 15 kb, 20 kb, 25 kb, or 30 kb in length.

In some embodiments the p8 protein is overexpressed from the canonical T7 promoter or a variant thereof. Overexpression of genes II, X, IV, I, XI and especially VIII from canonical T7 promoters increase production of phage particles and cssDNA. Decreased expression of gene V also increases production of the same.

Synthetic promoters can be characterized by the amount of RNA transcription that they can produce. Promoters that produce a greater amount of RNA transcripts are characterized as high, promoters that produce somewhat less can be described as medium and promoters that comparatively produce even less can be described as low. Examples of T7 promoters that fall into these categories include; High
(Taatacgactcactatagggcgaattgggtaccgggcccagaccacaacggtttccctctagaaataattttgtttaactttaagaaggagatatacat—SEQ ID NO: 1 (wild type T7));

### Medium

(taatccgactcactatagggcgaattgggtaccgggcccagaccacaacggtttccctctagaaataattttgtttaactttaagaaggagatatacat—SEQ ID NO: 2 (see sequence 7834, Komura et al., High-throughput evaluation of T7 promoter variants using biased randomization and DNA barcoding, Plos, 2018));

### Low

(Gaatacgactcactatagcgcgaattgggtaccgggcccagaccacaacggtttccctctagaaataattttgtttaactttaagaaggagatatacat—SEQ ID NO: 3 (see sequence 7812, Komura et al., High-throughput evaluation of T7 promoter variants using biased randomization and DNA barcoding, Plos, 2018))

Production strains can include at least two coding sequences that produce phage proteins that facilitate the production of cssDNA upon introduction of the phagemid. The at least two phage proteins that are integrated into the genome of the production strain are selected to optimize the yield and/or fidelity of the resulting cssDNA.

In some embodiments, a production strain includes a genomically integrated coding sequence for the p1 phage protein (SEQ ID NO: 4:
MAVYFVTGKLGSGKTLVSVGKIQDKIVAGCKIATNLDLRLQNLPQVGRFAKTPRVLRIPDKPSIS DLLAIGRGNDSYDENKNGLLVLDECGTWFNTRSWNDKERQPIIDWFLHARKLGWDIIFLVQDLSI VDKQARSALAEHVVYCRRLDRITLPFVGTLYSLITGSKMPLPKLHVGVVKYGDSQLSPTVERWL YTGKNLYNAYDTKQAFSSNYDSGVYSYLTPYLSHGRYFKPLNLGQKMKLTKIYLKKFSRVLCL AIGFASAFTYSYITQPKPEVKKVVSQTYDFDKFTIDSSQRLNLSYRYVFKDSKGKLINSDDLQKQG YSLTYIDLCTVSIKKGNSNEIVKCN). In some embodiments, the p1 coding sequence is under the control of an inducible promoter, such as a temperature sensitive or chemically sensitive promoter. In some embodiments, the second of the at least two coding sequences is selected from the following phage proteins: p2, p3, p4, p5, p6, p7, p8, p9, p10, and p11. In additional embodiments, the at least two coding sequences are integrated into the genome at distinct loci. This decreases the possibility of recombination and the formation of a replication competent phage.

In yet additional embodiments, the p1 coding sequence, and the second of the at least two coding sequences that is selected from phage proteins: p2, p3, p4, p5, p6, p7, p8, p9, p10, and p11 are placed under the control of the same promoter so that they are expressed at substantially the same time during production. In some embodiments the p1 and the second of the at least two coding sequences that is selected from phage proteins: p2, p3, p4, p5, p6, p7, p8, p9, p10, and p11 are genomically integrated at the same loci and a third phage protein coding sequence is genomically integrated at a distinct loci. In some embodiments a helper plasmid or helper virus is used to deliver any additional phage proteins necessary for cssDNA production.

In some embodiments, a production strain includes a genomically integrated coding sequence for the p2 phage protein (SEQ ID NO: 5:
MIDMLVLRLPFIDSLVCSRLSGNDLIAFVDLSKIATLSGMNLSARTVEYHIDGDLTVSGLSHPFESL PTHYSGIAFKIYEGSKNFYPCVEIKASPAKVLQGHNVFGTTDLALCSEALLLNFANSLPCLYDLLD VNATTISRIDATFSARAPNENIAKQVIDHLRNVSNGQTKSTRSQNWESTVTWNETSRHRTLVAYL KHVELQHQIQQLSSKPSAKMTSYQKEQLKVLSNPDLLEFASGLVRFEARIKTRYLKSFGLPLNLF DAIRFASDYNSQGKDLIFDLWSFSFSELFKAFEGDSMNIYDDSA VLDAIQSKHFTITPSGKTSFAK ASRYFGFYRRLVNEGYDSVALTMPRNSFWRYVSALVECGIPKSQLMNLSTCNNVVPLVRFINVD FSSQRPDWYNEPVLKIA). In some embodiments, the p2 coding sequence is under the control of an inducible promoter, such as a temperature sensitive or chemically sensitive promoter. In some embodiments the second of the at least two coding sequences is selected from the following phage proteins: p1, p3, p4, p5, p6, p7, p8, p9, p10, and p11. In additional embodiments, the at least two coding sequences are integrated into the genome at distinct loci. This decreases the possibility of recombination and the formation of a replication competent phage.

In yet additional embodiments, the p2 coding sequence, and the second of the at least two coding sequences that is selected from phage proteins: p1, p3, p4, p5, p6, p7, p8, p9, p10, and p11 are placed under the control of the same promoter so that they are expressed at substantially the same time during production. In some embodiments the p2 and the second of the at least two coding sequences that is selected from phage proteins: p1, p3, p4, p5, p6, p7, p8, p9, p10, and p11 are genomically integrated at the same loci and a third phage protein coding sequence is genomically integrated at a distinct loci. In some embodiments, a helper plasmid or helper virus is used to deliver any additional phage proteins necessary for cssDNA production.

In some embodiments, a production strain includes a genomically integrated coding sequence for the p3 phage protein (SEQ ID NO: 06:
MKKLLFAIPLVVPFYSHSAETVESCLAKPHTENSFTNVWKDDKTLDRYANYEGCLWNATGVVV CTGDETQCYGTWVPIGLAIPENEGGGSEGGGSEGGGSEGGGTKPPEYGDTPIPGYTYINPLDGTY PPGTEQNPANPNPSLEESQPLNTFMFQNNRFRNRQGALTVYTGTVTQGTDPVKTYYQYTPVSSK AMYDAYWNGKFRDCAFHSGFNEDPFVCEYQGQSSDLPQPPVNAGGGSGGGSGGGSEGGGSEG GGSEGGGSEGGGSGGGSGSGDFDYEKMANANKGAMTENADENALQSDAKGKLDSVATDYGA AIDGFIGDVSGLANGNGATGDFAGS). In some embodiments a p3 coding sequence is under the control of an inducible promoter, such as a temperature sensitive or chemically sensitive promoter. In some embodiments the second of the at least two coding sequences is selected from the following phage proteins: p1, p2, p4, p5, p6, p7, p8, p9, p10, and p11. In additional embodiments the at least two coding sequences are integrated into the genome at distinct loci. This decreases the possibility of recombination and the formation of a replication competent phage.

In yet additional embodiments, a p3 coding sequence, and the second of the at least two coding sequences that is selected from phage proteins: p1, p2, p4, p5, p6, p7, p8, p9, p10, and p11 are placed under the control of the same promoter so that they are expressed at substantially the same time during production. In some embodiments, the p3 and the second of the at least two coding sequences that is selected from phage proteins: p1, p2, p4, p5, p6, p7, p8, p9, p10, and p11 are genomically integrated at the same loci and a third phage protein coding sequence is genomically integrated at a distinct loci. In some embodiments, a helper plasmid or helper virus is used to deliver any additional phage proteins necessary for cssDNA production.

In some embodiments, a production strain includes a genomically integrated coding sequence for the p4 phage protein (SEQ ID NO: 7:
MKLLNVINFVFLMFVSSSSFAQVIEMNNSPLRDFVTWYSKQSGESVIVSPDVKGTVTVYSSDVKP ENLRNFFISVLRANNFDMVGSIPSIIQKYNPNNQDYIDELPSSDNQEYDDNSAPSGGFFVPQNDNV TQTFKINNVRAKDLIRVVELFVKSNTSKSSNVLSIDGSNLLVVSAPKDILDNLPQFLSTVDLPTDQI LIEGLIFEVQQGDALDFSFAAGSQRGTVAGGVNTDRLTSVLSSAGGSFGIFNGDVLGLSVRALKT NSHSKILSVPRILTLSGQKGSISVGQNVPFITGRVTGESANVNNPFQTIERQNVGISMSVFPVAMA GGNIVLDITSKADSLSSSTQASDVITNQRSIATTVNLRDGQTLLLGGLTDYKNTSQDSGVPFLSKIP LIGLLESSRSDSNEESTLYVLVKATIVRAL). In some embodiments, a p4 coding sequence is under the control of an inducible promoter, such as a temperature sensitive or chemically sensitive promoter. In some embodiments, the second of the at least two coding sequences is selected from the following phage proteins: p1, p2, p3, p5, p6, p7, p8, p9, p10, and p11. In additional embodiments, the at least two coding sequences are integrated into the genome at distinct loci. This decreases the possibility of recombination and the formation of a replication competent phage.

In yet additional embodiments, the p4 coding sequence and the second of the at least two coding sequences that is selected from phage proteins: p1, p2, p3, p5, p6, p7, p8, p9, and p10 are placed under the control of the same promoter so that they are expressed at substantially the same time during production. In some embodiments, the p4 and the second of the at least two coding sequences that is selected from phage proteins: p1, p2, p3, p5, p6, p7, p8, p9, p10, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p5 phage protein (SEQ ID NO: 08:
MIKVEIKPSQAQFTTRSGVSRQGKPYSLNEQLCYVDLGNEYPVLV
KITLDEGQPAYAPGLYTVHLSSFKVGQFGSLMIDRLRLVPAK). In some embodiments, the p5 coding sequence is under the control of an inducible promoter, such as a temperature sensitive or chemically sensitive promoter. In some embodiments, the second of the at least two coding sequences is selected from the following phage proteins: p1, p2, p3, p4, p6, p7, p8, p9, p10, and p11. In additional embodiments, the at least two coding sequences are integrated into the genome at distinct loci. This decreases the possibility of recombination and the formation of a replication competent phage.

In yet additional embodiments, the p5 coding sequence, and the second of the at least two coding sequences that is selected from phage proteins: p1, p2, p3, p4, p6, p7, p8, p9, p10, and p11 are placed under the control of the same promoter so that they are expressed at substantially the same time during production. In some embodiments, the p5 and the second of the at least two coding sequences that is selected from phage proteins: p1, p2, p3, p4, p6, p7, p8, p9, p10, and p11 are genomically integrated at the same loci and a third phage protein coding sequence is genomically integrated at a distinct loci. In some embodiments, a helper plasmid or helper virus is used to deliver any additional phage proteins necessary for cssDNA production.

In some embodiments, a production strain includes a genomically integrated coding sequence for the p6 phage protein (SEQ ID NO: 9:
MPVLLGIPLLLRFLGFLLVTLFGYLLTFLKKGFGKIAIAISLFLALIIGLNSILVGYLSDISAQLPSDF VQGVQLILPSNALPCFYVILSVKAAIFIFDVKQKIVSYLDWDK). In some embodiments, the p6 coding sequence is under the control of an inducible promoter, such as a temperature sensitive or chemically sensitive promoter. In some embodiments, the second of the at least two coding sequences is selected from the following phage proteins: p1, p2, p3, p4, p5, p7, p8, p9, p10, and p11. In additional embodiments, the at least two coding sequences are integrated into the genome at distinct loci. This decreases the possibility of recombination and the formation of a replication competent phage.

In yet additional embodiments, the p6 coding sequence, and the second of the at least two coding sequences that is selected from phage proteins: p1, p2, p3, p4, p5, p7, p8, p9, and p10 are placed under the control of the same promoter so that they are expressed at substantially the same time during production. In some embodiments, the p6 and the second of the at least two coding sequences that is selected from phage proteins: p1, p2, p3, p4, p5, p7, p8, p9, p10, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p7 phage protein (SEQ ID NO: 10: MEQVADFDTIYQAMIQISVVLCFALGIIAGGQR). In some embodiments, the p7 coding sequence is under the control of an inducible promoter, such as a temperature sensitive or chemically sensitive promoter. In some embodiments, the second of the at least two coding sequences is selected from the following phage proteins: p1, p2, p3, p4, p5, p6, p8, p9, and p10. In additional embodiments, the at least two coding sequences are integrated into the genome at distinct loci. This decreases the possibility of recombination and the formation of a replication competent phage.

In yet additional embodiments, the p7 coding sequence, and the second of the at least two coding sequences that is selected from phage proteins: p1, p2, p3, p4, p5, p6, p8, p9, p10, and p11 are placed under the control of the same promoter so that they are expressed at substantially the same time during production. In some embodiments, the p7 and the second of the at least two coding sequences that is selected from phage proteins: p1, p2, p3, p4, p5, p6, p8, p9, p10, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p8 phage protein (SEQ ID NO: 11:
MKKSLVLKASVAVATLVPMLSFAAEGDDPAKAAFNSLQASATEYIGYAWAMVVVIVGATIGIK LFKKFTSKAS). In some embodiments, the p8 coding sequence is under the control of an inducible promoter, such as a temperature sensitive or chemically sensitive promoter. In some embodiments, the second of the at least two coding sequences is selected from the following phage proteins: p1, p2, p3, p4, p5, p6, p7, p9, p10, and p11. In additional embodiments, the at least two coding sequences are integrated into the genome at distinct loci. This decreases the possibility of recombination and the formation of a replication competent phage.

In yet additional embodiments, the p8 coding sequence and the second of the at least two coding sequences that is selected from phage proteins: p1, p2, p3, p4, p5, p6, p7, p9, p10, and p11 are placed under the control of the same promoter so that they are expressed at substantially the same time during production. In some embodiments, the p8 and the second of the at least two coding sequences that is selected from phage proteins: p1, p2, p3, p4, p5, p6, p7, p9, p10, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p9 phage protein (SEQ ID NO: 12: MSVLVYSFASFVLGWCLRSGITYFTRLMETSS). In some embodiments, the p9 coding sequence is under the control of an inducible promoter, such as a temperature sensitive or chemically sensitive promoter. In some embodiments, the second of the at least two coding sequences is selected from the following phage proteins: p1, p2, p3, p4, p5, p6, p7, p8, p10, and p11. In additional embodiments, the at least two coding sequences are integrated into the genome at distinct loci. This decreases the possibility of recombination and the formation of a replication competent phage.

In yet additional embodiments, the p9 coding sequence and the second of the at least two coding sequences that is selected from phage proteins: p1, p2, p3, p4, p5, p6, p7, p8, p10, and p11 are placed under the control of the same promoter so that they are expressed at substantially the same time during production. In some embodiments, the p9 and the second of the at least two coding sequences that is selected from phage proteins: p1, p2, p3, p4, p5, p6, p7, p8, p10, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p10 phage protein (SEQ ID NO: 13:
MNIYDDSAVLDAIQSKHFTITPSGKTSFAKASRYFGFYRRLVNEGYDSVALTMPRNSFWRYVSA LVECGIPKSQLMNLSTCNNVVPLVRFINVDFSSQRPDWYNEPVLKIA). In some embodiments, the p10 coding sequence is under the control of an inducible promoter, such as a temperature sensitive or chemically sensitive promoter. In some embodiments, the second of the at least two coding sequences is selected from the following phage proteins: p1, p2, p3, p4, p5, p6, p7, p8, p9, and p11. In additional embodiments, the at least two coding sequences are integrated into the genome at distinct loci. This decreases the possibility of recombination and the formation of a replication competent phage.

In yet additional embodiments, the p10 coding sequence and the second of the at least two coding sequences that is selected from phage proteins: p1, p2, p3, p4, p5, p6, p7, p8, p9, and p11 are placed under the control of the same promoter so that they are expressed at substantially the same time during production. In some embodiments, the p10 and the second of the at least two coding sequences that is selected from phage proteins: p1, p2, p3, p4, p5, p6, p7, p8, p9, and p11 are genomically integrated at the same loci and a third phage protein coding sequence is genomically integrated at a distinct locus. In some embodiments, a helper plasmid or helper virus is used to deliver any additional phage proteins necessary for cssDNA production.

In some embodiments, a production strain includes a genomically integrated coding sequence for the p11 phage protein (SEQ ID NO: 14:
MKLTKIYLKKFSRVLCLAIGFASAFTYSYITQPKPEVKKVVSQTYDFDKFTIDSSQRLNLSYRYVF KDSKGKLINSDDLQKQGYSLTYIDLCTVSIKKGNSNEIVKCN). In some embodiments, the p11 coding sequence is under the control of an inducible promoter, such as a temperature sensitive or chemically sensitive promoter. In some embodiments, the second of the at least two coding sequences is selected from the following phage proteins: p1, p2, p3, p4, p5, p6, p7, p8, p9, and p10. In additional embodiments, the at least two coding sequences are integrated into the genome at distinct loci. This decreases the possibility of recombination and the formation of a replication competent phage.

In yet additional embodiments, the p11 coding sequence and the second of the at least two coding sequences that is selected from phage proteins: p1, p2, p3, p4, p5, p6, p7, p8, p9, and p10 are placed under the control of the same promoter so that they are expressed at substantially the same time during production. In some embodiments, the p11 and the second of the at least two coding sequences that is selected from phage proteins: p1, p2, p3, p4, p5, p6, p7, p8, p9, and p10 are genomically integrated at the same loci and a third phage protein coding sequence is genomically integrated at a distinct locus. In some embodiments, a helper plasmid or helper virus is used to deliver any additional phage proteins necessary for cssDNA production.

### Example 1: M13 Whole Genome Integration into Production Strain

The M13 genome is organized in two separate transcriptional units. The first consists of genes II, X, V, VII, IX and VIII and is expressed from strong promoters. The second consists of genes III, VI, I, XI and IV and is expressed from weak promoters. Two of the genes overlap with the open reading frames of two other genes (e.g. the gene for p10 is completely contained within the coding region for p2.) PA, PB, PH are strongly active promoters (see FIG. 1B). Two additional weak promoters are labeled PZ and PW. Two terminators (T) are strong, rho-independent terminators; the third, weaker terminator (T(weak)) is rho-dependent.

Smeal et al., Simulation of the M13 life cycle I: Assembly of a genetically-structured deterministic chemical kinetic simulation, Virology, Volume 500, 2017, Pages 259-274.

In order to create a stable cell line capable of producing M13 phage particles while also being highly unlikely to recombine with a phagemid to produce a replication-competent phage, each transcriptional unit of the M13 bacteriophage containing protein coding genes and lacking a phage origin of replication and phage packaging signal is cloned from the publicly available helper phage M13KO7 (www.neb.com/products/n0315-M13KO7-helper-phage) (SEQ ID NO: 24) and integrated into the E. coli MG1655 genome at two loci.

The first transcriptional unit is cloned into a circular, double-stranded plasmid along with homology arms for integration into the E. coli MG1655 genome at the pyrF locus, a spectinomycin selectable marker, to allow for selection of integrants, and a p15A origin for replication in E. coli. The selectable marker is flanked by loxP sites to allow marker recycling upon expression of the cre gene. The insert containing homology arms, M13 protein coding genes and a selectable marker is flanked by restriction enzyme sites so that the circular double-stranded DNA can be linearized prior to transformation of E. coli. The plasmid is digested with the restriction enzymes and then electrophoresed on a 1% agarose tris-acetate-ethylenediaminetetraacetic acid (TAE) gel to separate the desired integration cassette from the undesired plasmid backbone containing an p15A E. coli origin of replication. The band containing the integration cassette is purified using the Qiagen Gel Extraction Kit (catalog number 28706 Qiagen, Hilden, Germany), following the manufacturer's recommended protocol. Since the p15A E. coli origin of replication, which is required for plasmid replication in E. coli, is selected against using agarose gel electrophoresis, one can be confident that any E. coli cells that survive the antibiotic selection after transformation with the integration cassette are likely to have gone through genomic integration events rather than transformation with a replicating plasmid.

### Creating a Recombineering Competent Host

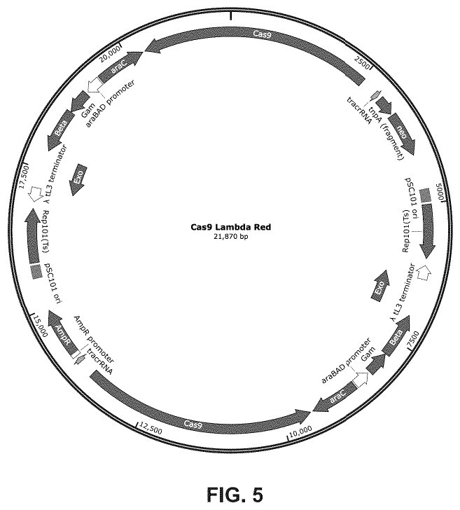

The E. coli host strain MG1655 is first transformed with a publicly available recombineering plasmid expressing the lambda red recombineering system including beta, exo and gamma from an arabinose inducible promoter (Catalog number CAS9BAC1P, Sigma Aldrich, Burlington, Massachusetts) (see FIG. 5).

The plasmid also expresses kanamycin antibiotic marker to allow for selection and maintenance of the plasmid in a population of bacterial cells and a temperature sensitive pSC101 replication factor, repA101ts to allow eviction of the plasmid when expression of the lambda red recombineering system is no longer needed. Transformants are selected by plating on Lysogeny Broth agar (LB) (Bertani, G. 1952. “Studies on Lysogenesis. I. The mode of phage liberation by lysogenic Escherichia coli.” J. Bacteriology, 62:293-300) agar supplemented with 50 ug/ml kanamycin. Colonies are picked and inoculated into 15 ml LB liquid medium supplemented with 50 ug/ml kanamycin to maintain the plasmid. The cultures are incubated overnight at 30 C. 1 ml of the overnight culture is mixed with 1 ml of 50% glycerol/50% water solution and stored at −80 C to create a permanent bank of a new strain referred to as the MG1655 recombineering strain. The plasmid DNA is isolated from the remaining 14 ml of the cultures and sequenced via Oxford Nanopore technology to confirm presence and sequence identity of the recombineering plasmid.

### Inducing Expression of the Recombineering Genes

Once the transformation of the E. coli MG1655 strain with the recombineering plasmid is confirmed, the recombineering strain is struck out on LB agar supplemented with 50 ug/ml kanamycin and incubated overnight at 30 C. A single colony is then inoculated into LB Lennox (low salt formulation) supplemented with 50 ug/ml kanamycin and incubated overnight at 30 C. The culture will then be diluted to OD 0.01 in LB liquid medium supplemented with 50 ug/ml kanamycin. The culture will be incubated at 30 C until it reaches OD 0.30. At that point, it will be diluted 1:2 with pre-warmed LB broth supplemented with 4% L-arabinose will to create a final concentration of 2% L-arabinose to induce expression of the recombineering genes beta, exo and gamma. The culture will then be incubated at 30 C for an additional hour to allow sufficient amounts of the recombineering proteins beta, exo and gamma to accrue in the cells.

Electroporation with the Phage Genome Integration Cassette

After one hour of recombineering gene expression, cells from 1 ml of the culture will be harvested by centrifugation for one minute at 7000 RCF and then washed three times in ice-cold, deionized water to remove salts. Cells will then be re-suspended in 49 μl ice-cold, deionized water and 1 μl of the gel-purified, linearized integration cassette consisting of 2 pyrF homology arms, all the protein coding sequences from the first transcriptional unit of M13KO7 and a selectable marker flanked by loxP sites.

The mixture of cells and DNA will be electroporated using a Bio-Rad Gene Pulser with a 1 mm cuvette, 2.1 kv, 100 ohms resistance and 25 microFarrads capacitance. Immediately following application of the electrical pulse, cells will be re-suspended in 1 ml LB Lennox medium and incubated overnight at 30 C. Following this overnight recovery period, cells will be plated on LB agar supplemented with 50 ug/ml spectinomycin to select for integration events.

Identification of Cells with Correct Integration Events

Plates will be incubated overnight to allow colonies to form. Colonies will be screened by PCR to identify correct colonies formed from correct integration events. Three pairs of PCR primers will be used. The first will check the upstream end of the integration. The forward primer will bind in the genomic region upstream of the sequence found in the upstream homology arm and point in towards the integration cassette. The reverse primer will bind in the M13 genes in the integration cassette and point out towards the E. coli genomic region upstream of the desired integration event.

The second primer pair will check the downstream end of the integration. The forward primer will bind in the M13 genes in the integration cassette and point out towards the E. coli genomic region downstream of the desired integration. The reverse primer will bind in the E. coli genome downstream of the downstream homology arm sequence and point in toward the integration cassette. If both the first and second pair of primes yield a PCR product of the expected size, then it can be assumed that some cells in the colony screened have the desired integration event.

The third primer pair will consist of the forward (genomic) primer from the first pair and the reverse (genomic) primer from the second pair. If these primers yield a PCR product of 8 kb encompassing the pyrF homology arms, the protein coding region of M13KO7 and the spectinomycin marker then it can be assumed that some cells in the colony have the correct integration event. If these primers yield a product of 2 kb encompassing only the pyrF homology arms and the pyrF gene, then it can be assumed that some cells in the colony retain the wild type pyrF sequence.

Correct colonies will be ones that yield PCR products using all three primer pairs but that lack any evidence of the smaller, 2 kb PCR product with the third pair of primers. A subset of these correct colonies are inoculated into LB supplemented with 50 ug/ml spectinomycin and grown overnight. 1 ml of the overnight culture is mixed with 1 ml of a 50% glycerol/50% water solution and stored at −80° C. to create a permanent bank of the new strain. Genomic DNA is extracted from another 1 ml of the overnight culture using the Sigma GenElute Genomic Prep Kit (Catalog #NA2120, Sigma Aldrich, Burlington, Massachusetts). This purified genomic DNA is sequenced by Azenta, Inc. (Chelmsford, Massachusetts), using standard Illumina Next Generation sequencing instruments and protocols. The fastQ files produced by Azenta are aligned to both the expected sequence at the integration locus and to the wild type sequence at the same locus using the built-in mapping algorithm within Geneious Prime (Copyright © 2005-2022 Biomatters Ltd.). Samples where no reads align to the wild type pyrF gene and where some reads align to all of the protein coding sequences of the first transcriptional unit of M13KO7 without any sequence discrepancies will be deemed correct. The glycerol stock associated with one of these correct samples will be chosen for future steps and the others will be discarded.

### Eviction of the Recombineering Plasmid

The resulting E. coli strain with a correct integration of all protein coding genes from the first transcriptional unit of M13 ko7 and a deletion of pyrF is struck out on LB agar supplemented with 50 ug/ml spectinomycin and incubated overnight at 37 C. Notably, the Lb agar lacks kanamycin which had been used to maintain the recombineering plasmid. A single colony is inoculated into LB broth supplemented with 50 ug/ml spectinomycin and incubated overnight at 42 C in order to prevent the temperature sensitive pSC101 replication factor from initiating replication of the plasmid. A dilution series of this culture is plated on LB agar supplemented with 50 ug/ml spectinomycin in order to obtain colonies founded by single cells. These plates will be incubated overnight at 37 C and the resulting colonies will be patched onto both LB agar supplemented with 50 ug/ml spectinomycin and LB agar supplemented with 50 ug/ml spectinomycin and 50 ug/ml kanamycin. Colonies that grow only on the former and not on the latter plate will be assumed to lack the recombineering plasmid. One of these plasmid-free colonies will be inoculated into LB broth supplemented with 50 ug/ml spectinomycin and incubated overnight at 37 C. A glycerol stock is stored at −80 C.

### Antibiotic Marker Recycling

The plasmid-free overnight culture above, is diluted to OD 0.01 and incubated at 37 C until it achieves OD 0.30. One ml of the culture is then harvested and washed three times in ice-cold deionized water by centrifugation at 7000 RCF for 1 minute. The cells are then re-suspended in 49 μl ice-cold deionized water and mixed with 1 μl plasmid DNA encoding the cre gene, the pSC101ts origin of replication and a kanamycin marker. The mixture of washed cells and DNA is then electroporated as described above, re-suspended in 1 ml LB broth and incubated at 37 C for 1 hour. The cells are then plated on LB supplemented with 50 ug/ml kanamycin to select for cells that have been transformed with the cre expression plasmid. The plates are incubated overnight at 37 C. Resulting colonies are patched onto both LB supplemented with 50 ug/ml kanamycin and LB supplemented with 50 ug/ml kanamycin and 50 ug/ml spectinomycin. Colonies that grow on the former plate but not on the latter can be assumed to have recycled the spectinomycin marker through cre-induced recombination at the loxP markers that had flanked the spectinomycin gene. This can be verified through colony PCR with primers that bind to the integration cassette outside of the loxP recombination sites and point in towards the region where the spectinomycin had been. A 1 kb amplicon would indicate that the spectinomycin gene was still in place, but a shorter amplicon would indicate that it had been lost through recombination. The lambda red recombinase system and the cre/lox marker recycling system was described by Tuntufye and Goddeeris, FEMS Microbiol Lett 325 (2011) 140-147, which is herein incorporated by reference in its entirety.

### Eviction of the Cre Expression Plasmid

One colony that is verified to have lost the spectinomycin resistance gene through both lack of growth on spectinomycin plates and through colony PCR is inoculated into LB broth without antibiotics and incubated overnight at 42 C to prevent the temperature sensitive pSC101 replication factor from initiating replication of the cre expression plasmid. A dilution series of this culture is plated on LB agar without antibiotics and incubated overnight at 37 C to obtain colonies founded by single cells. Colonies are patched onto both LB without antibiotics and LB supplemented with 50 ug/ml kanamycin to check for loss of the cre expression plasmid. Colonies that grow on the former plate, but not on the latter can be presumed to have evicted the plasmid. One such colony is inoculated into LB without antibiotics and incubated overnight at 37 C. The resulting culture is mixed one to one with a 50% glycerol/50% water solution, labeled as KTx00001 and stored at −80 C.

### Integration of the Second Transcriptional Unit

The steps above are repeated for the integration of the second transcriptional unit. The only changes are in the sequence of the transcriptional unit, the homology arms and primers used to verify the integration. The second transcriptional unit is integrated at the neutral integration locus, yhiN in E. coli MG1655, Bernhards et al., ACS Synth. Biol. 2022, 11, 1681-1685. This new strain is labeled KTx00002 and stored at −80.

### Testing the New Strain

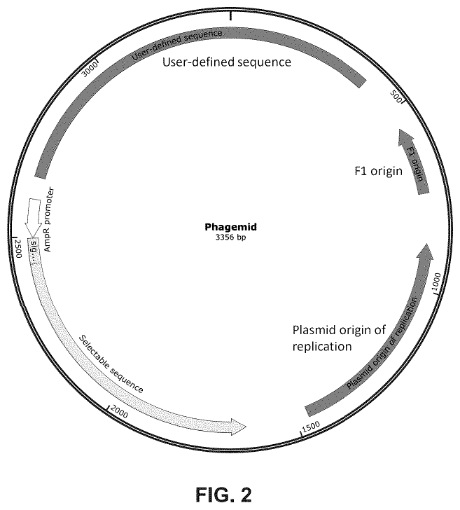

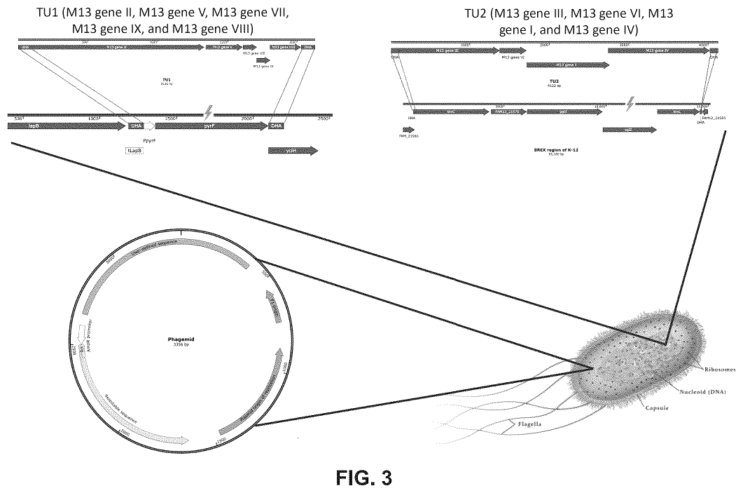

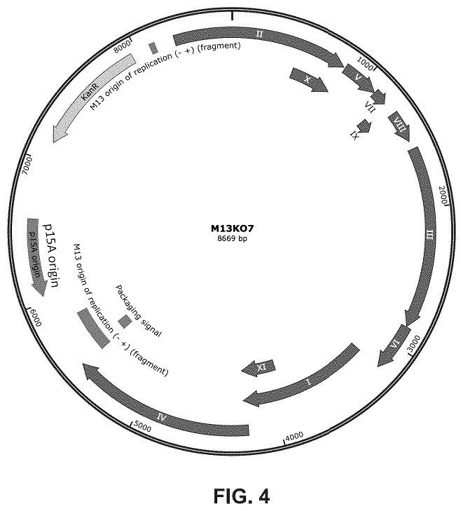

Confirmation that KTx00002 can produce cssDNA upon infection with a phagemid (FIG. 2) that contains a F1 ori, packaging signal, and a selectable sequence in the form of an ampicillin resistance gene is accomplished by electroporation of the phagemid into KTx00002 followed by cssDNA quantification as follows.

The phagemid is transformed into KTx00002 using a standard electroporation protocol. In short, the cells are grown to OD 0.3, then one ml of culture is harvested by centrifugation for 1 minute at 6000 RCF. The pellet is washed 3 times in ice-cold, deionized water and the cells are re-suspended in 49 μl of deionized water. 1 μl of phagemid DNA is added to the cells which are then mixed by flicking the tube. The cells and DNA are transferred to a 1 mm cuvette and electroporated in a Bio Rad Gene Pulser at 2.1 kv/cm, 100 ohms and 25 uF. The cells are allowed to recover in SOC medium for 1 hour at 37 C with shaking before they are washed and plated on defined medium lacking uracil. (Bacto CD Supreme Fermentation Production Medium (FPM) Catalog number A49737-01, Thermo Fisher, Waltham, MA, USA). Plates are incubated overnight at 37 C. Single colonies are picked and inoculated into 4 ml FPM lacking uracil and grown overnight at 30 C. In the morning, the optical density (OD) is measured and the culture is diluted to OD 0.05 in 750 ml FPM lacking uracil. This culture is then incubated for 8 hours at 30 C.

The culture is centrifuged to remove whole cells and the phage particles are collected in the supernatant. Phage particles are purified from the fermentation medium by polyethylene glycol and sodium chloride precipitation (3% weight by volume each) followed by centrifugation at 5000 g. The phage particle pellet is then lysed using a solution of 1% sodium dodecyl sulfate and 200 mM sodium hydroxide. The lysed phage particle debris is pelleted by centrifugation at 12,000 g and the supernatant containing the cssDNA is retained. Endotoxin lipopolysaccharides are removed by adding triton X-100 detergent, mixing thoroughly and then discarding the resulting detergent layer. The cssDNA is then precipitated by adding 100% ethanol at −20 degrees C. to the solution, incubating on ice for a half hour, and then pelleting by centrifugation at 12,000 g. The cssDNA pellet is then washed with 75% ethanol at 4 C, incubated on ice for 20 minutes, and centrifuged again at 12,000 g to remove residual salts. The resulting cssDNA pellet is dried to remove residual ethanol and re-suspended in a buffered solution of tris-EDTA (TE) or nuclease-free water.

cssDNA is quantitated using the Qubit commercial assay kit and instrument following the manufacturers recommended protocol. Nucleic acid purity is quantitated using an Agilent Bioanalyzer. Endotoxin levels are quantitated via the Endosafe® nexgen-PTS™ handheld spectrophotometer and cartridge assay (Catalog number MCS150K, Charles River Laboratories, Wilmington, MA, USA) following the manufacturer's suggested protocol which utilizes a kinetic limulus amoebocyte lysate assay according to USP BET<85> and EP BET<2.6.14> criver.com/sites/default/files/resources/Endosafe-PTS-Regulatory-Requirements-USPBET85-EPBET2.6.14.pdf.

In comparison, the parental strain (E. coli MG1655) of KTx0002 is similarly transformed with the same phagemid and a helper plasmid which is derived from helper phage M13KO7 by removing the F1 origin and the packaging signal by amplifying the kanamycin marker and all of the protein coding genes from M13KO7 with primers designed to exclude the F1 origin of replication and the packaging signal and then re-circularizing the amplified DNA using a DNA ligase in vitro reaction. It is believed that the KTx00002 will produce cssDNA at a similar yield and fidelity compared to the parental control.

### Example 2: Impact of Induced Gene Expression on KTx00002

Strain KTx00002 includes integrated copies of the two transcriptional units of M13KO7 and when transformed with a phagemid, achieves cssDNA production similar to its parent strain, MG1655 transformed with the M13KO7 helper plasmid and the same phagemid. In order to achieve high cssDNA production, certain genes from M13KO7 are overexpressed.

To overexpress M13KO7 genes in E. coli, first the T7 polymerase coding sequence is integrated into the E. coli genome at the neutral integration locus yjhV. This is accomplished in the same manner that the two transcriptional units of M13KO7 were integrated. Briefly, the T7 polymerase gene is codon optimized for expression in E. coli and synthesized by Genscript (No. 28 Yongxi Road, Jiangning District, Nanjing, Jiangsu, China 211100). It is cloned into a plasmid such that it is under the control of the lactose inducible promoter pLac. Downstream of this pLac-T7 polymerase expression cassette is the pheA transcriptional terminator, and a spectinomycin marker flanked by loxP sites. Both the expression cassette and the spectinomycin marker are flanked by homology arms and restriction enzyme sites. This plasmid is extracted from its E. coli host using the QIAprep Spin Miniprep Kit (catalog number 27106 Qiagen, Hilden, Germany) and digested with restriction enzymes to linearize the vector creating an integration cassette that is separate from the bacterial origin of replication. KTx00002 is transformed with the recombineering plasmid as above, grown to OD 0.3 at which point the expression of the recombineering genes is induced with L-arabinose. The cells are incubated for an additional hour to allow expression of the recombineering genes. The cells are then harvested and washed 3 times by centrifugation and mixed with 1 μl of the linearized integration cassette before being electroporated. Cells are allowed to recover overnight then are plated on LB agar supplemented with 50 ug/ml spectinomycin to select for the integration cassette. Integration is verified through colony PCR and sequencing as above. The recombineering plasmid is evicted as described above. The cells are transformed with the cre expression plasmid as described above and the marker is recycled as above. Lastly the cre expression plasmid is evicted and the new strain labeled KTx00003 is stored at −80.

There is evidence that over-expression of certain M13 phage genes leads to increased production of cssDNA in E. coli. Behler et al., 2022 October; 119(10):2878-2889. The gene 2 and 10 open reading frame involved in expression of phage genes, the gene 1 and 11 open reading frame involved in creating pores in bacterial membranes for phage secretion, gene 4 also involved in creating secretion pores and gene 8 the major coat protein. However, this evidence is not derived from genomically integrated expression of the phage genes.

Over-expression of the phage genes individually and in combinations is accomplished by cloning the coding sequences under the control of a T7 promoter into a plasmid that contains a transcriptional terminator downstream of the gene or genes, followed by a spectinomycin selectable marker flanked by loxP sites. Both the phage gene expression cassette and the spectinomycin marker are flanked by homology arms for integration into the E. coli genome at neutral integration loci. The homology arms are flanked by restriction enzyme recognition sequences so the vector can be linearized prior to integration into the E. coli genome.

These plasmids are prepped and digested as described above and sequentially integrated into the E. coli KTx00003 genome using the recombineering and cre/lox system described above to create a new E. coli strain which contains the two wild type transcriptional units of M13KO7 plus the T7 polymerase under the control of the lac promoter and the new overexpression cassettes that include T7 promoters driving the expression of phage genes as shown in Table 1. The new strains are referred to using the strain designations KTx00004.X.Y, where when “X” is a 1 the code refers to an expression cassette that overexpresses p1, and where when “X” is a 2 the code refers to an expression cassette that overexpresses p2 . . . and so on for each of genes I-XI. Similarly, when two different phage genes are overexpressed the “Y” is used to identify the second phage gene that is overexpressed. The Table below presents the various combinations with the X.Y identified.

TABLE 1

| KTx00004.X.Y. | p1 | p2 | p3 | p4 | p5 | p6 | p7 | p8 | p9 | p10 | p11 |
|---|---|---|---|---|---|---|---|---|---|---|---|
| p1 | 1 | 1.2 | 1.3 | 1.4 | 1.5 | 1.6 | 1.7 | 1.8 | 1.9 | 1.10 | 1.11 |
| p2 | 2.1 | 2 | 2.3 | 2.4 | 2.5 | 2.6 | 2.7 | 2.8 | 2.9 | 2.10 | 2.11 |
| p3 | 3.1 | 3.2 | 3 | 3.4 | 3.5 | 3.6 | 3.7 | 3.8 | 3.9 | 3.10 | 3.11 |
| p4 | 4.1 | 4.2 | 4.3 | 4 | 4.5 | 4.6 | 4.7 | 4.8 | 4.9 | 4.10 | 4.11 |
| p5 | 5.1 | 5.2 | 5.3 | 5.4 | 5 | 5.6 | 5.7 | 5.8 | 5.9 | 5.10 | 5.11 |
| p6 | 6.1 | 6.2 | 6.3 | 6.4 | 6.5 | 6 | 6.7 | 6.8 | 6.9 | 6.10 | 6.11 |
| p7 | 7.1 | 7.2 | 7.3 | 7.4 | 7.5 | 7.6 | 7 | 7.8 | 7.9 | 7.10 | 7.11 |
| p8 | 8.1 | 8.2 | 8.3 | 8.4 | 8.5 | 8.6 | 8.7 | 8 | 8.9 | 8.10 | 8.11 |
| p9 | 9.1 | 9.2 | 9.3 | 9.4 | 9.5 | 9.6 | 9.7 | 9.8 | 9 | 9.10 | 9.11 |
| p10 | 10.1 | 10.2 | 10.3 | 10.4 | 10.5 | 10.6 | 10.7 | 10.8 | 10.9 | 10 | 10.11 |
| p11 | 11.1 | 11.2 | 11.3 | 11.4 | 11.5 | 11.6 | 11.7 | 11.8 | 11.9 | 11.10 | 11 |

Overexpression of the phage genes from the T7 promoters is verified through reverse transcription-quantitative polymerase chain reaction (rt-qPCR). Cultures of the new strains and the parental control strain are grown overnight and then diluted to OD 0.01 and grown with and without the addition of 100 μM IPTG. Cells are harvested by centrifugation after 5 hours of growth. mRNA is prepped using the Qiagen RNeasy Mini kit (Catalog number 74104, Qiagen Hilden, Germany) following the manufacturer's suggested protocol. cDNA is reverse transcribed using TaqMan™ Reverse Transcription Reagents (Catalog number N8080234, ThermoFisher Scientific, Waltham, MA) and following the manufacturer's recommended protocol. cDNA preps are normalized to equal concentrations and amplified in multiplexed reactions with primers targeting each of the M13 genes and primers targeting the housekeeping control gene rpoD. Taqman probes (ThermoFisher Scientific, Waltham, MA) are added to the reactions to quantify the amount of each PCR product produced. For the M13 genes, the probes have a 5′ flourescein reporter dye, and 3′ Iowa Black® FQ quencher. For the rpoD housekeeping control gene, the probes have a 5′ cyanine-5 reporter dye and a 3′ Iowa Black® RQ quencher. Reactions are run in a QuantStudio 7 Flex (Thermofisher Scientific, Waltham, MA). Overexpression is determined by comparing the cycle at which the flourescein signal crosses an arbitrary threshold normalized to the cycle at which the cyanine-5 signal crosses the same threshold (the delta cycle threshold or Δct) compared to the wt parent and to the uninduced controls (the delta cycle threshold or ΔΔct). Strains expressing the M13 genes from T7 promoters cross the arbitrary threshold (normalized to the cyanine-5 signal) after fewer cycles than strains expressing the phage genes only from their native promoters.

cssDNA from KTx00004 is compared to cssDNA production from its parent strain KTx00002 which contains only the two wild type transcriptional units of M13KO7. For this comparison, each strain is transformed with the same phagemid. Cultures of the strains with the phagemid are grown side-by-side and cssDNA is extracted and quantified.

### Example 3: Differential Expression of Phage Genes in Production Host

For experimentation purposes, each phage gene is cloned into integration cassettes under the control of high, medium and low strength T7 promoters, see above. Each gene is integrated into the E. coli KTx0003 genome in parallel using the lambda red recombineering system described above to create 33 new strains each expressing one phage gene from one T7 promoter. The genes examined through the use of the plasmid include phage genes I, II, III, IV, V, VI, VII, VIII, IX, X and XI. The 33 new strains are tested for their ability to produce cssDNA.

In this experiment all genes (non-test genes) except for the test gene are placed under the control of their native promoter that can yield substantially the same amount of expression of the non-test genes as wild type phage expression levels. The single test gene is induced through addition of IPTG to activate expression of the T7 polymerase which in turn expresses the test genes from the T7 promoters. IPTG is added at various time points in the growth of the production host. The amount of cssDNA produced at the time points when the IPTG is introduced is quantified. For clarity, an example includes placing gene VIII under the control of the T7 promoter (SEQ ID NO: 1) and the remaining genes are placed under the control of their native promoters. Lactose or its analog IPTG is used to induce expression of the T7 polymerase which in turn transcribes gene VIII from the T7 promoter at OD 0.05, 0.5, 1.0, and 3.0. cssDNA yield and purity is determined using the Qubit in conjunction with the Bioanalyzer and the commercially available assay kits and protocols designed to work with those instruments.

To quantitate cssDNA produced from these test strains and the parental control strain KTx0003, the production hosts are transformed with the same phagemid and cultured in Bacto CD Supreme Fermentation Production Medium (FPM) (Catalog number A4973702 Thermo Fisher Scientific, Waltham, MA USA), media until it reaches the indicated O.D. using sterile media as the blank. At the indicated O.D., Isopropyl β-d-1-thiogalactopyranoside (IPTG) is added and the culture is returned to shaking at 200 RPM in a New Brunswick Innova shaking incubator. The culture is allowed to continue growing until it reaches 8 hours total growth time. The culture is spun down to remove whole cells and the phage particles are collected in the supernatant. Phage particles are purified from the fermentation medium by polyethylene glycol and sodium chloride precipitation (3% weight by volume each) followed by centrifugation at 5000 g. The phage particle pellet is then lysed using a solution of 1% sodium dodecyl sulfate and 200 mM sodium hydroxide. The lysed phage particle debris is pelleted by centrifugation at 12000 g and the supernatant containing the cssDNA is retained. The cssDNA is then precipitated by adding 100% ethanol at −20 degrees C. to the solution, incubating on ice for a half hour, and then pelleting by centrifugation at 12000 g. The cssDNA pellet is then washed in 75% 4 C ethanol, incubated on ice for 20 minutes, and spun down again at 12000 g to remove residual salts. The resulting cssDNA pellet is dried to remove ethanol and re-suspended in a buffered solution of TE or nuclease-free water.

cssDNA is quantitated using Qubit commercial assay kit and instrument following the manufacturers recommended protocol. Nucleic acid purity is quantitated using an Agilent Bioanalyzer. Endotoxin levels are quantitated via the Endosafe® nexgen-PTS™ handheld spectrophotometer and cartridge assay (Catalog number MCS150K, Charles River Laboratories, Wilmington, MA, USA) following the manufacturer's suggested protocol which utilizes a kinetic limulus amoebocyte lysate assay according to USP BET<85> and EP BET<2.6.14> www.criver.com/sites/default/files/resources/Endosafe-PTS-Regulatory-Requirements-USPBET85-EPBET2.6.14.pdf.

TABLE 2
List of strains

| Strain ID | Strain Description |
|---|---|
| KTx00001 | E. coli MG1655 with M13KO7 transcription |
| | unit 1 integrated at pyrF (uracil auxotroph) |
| KTx00002 | E. coli MG1655 with M13KO7 transcription |
| | unit 1 integrated at pyrF (uracil |
| | auxotroph) and M13KO7 transcription |
| | unit 2 integrated at yhiN |
| KTx00003 | E. coli MG1655 with M13KO7 transcription |
| | unit 1 integrated at pyrF (uracil |
| | auxotroph) and M13KO7 transcription |
| | unit 2 integrated at yhiN with pLac-T7 |
| | polymerase integrated at yjhV |
| KTx00004 | E. coli MG1655 with M13KO7 transcription |
| | unit 1 integrated at pyrF (uracil |
| | auxotroph) and M13KO7 transcription |
| | unit 2 integrated at yhiN with pLac-T7 |
| | polymerase integrated at yjhV |
| KTx00004.X.Y. | E. coli MG1655 with M13KO7 transcription |
| | unit 1 integrated at pyrF (uracil |
| | auxotroph) and M13KO7 transcription |
| | unit 2 integrated at yhiN with pLac-T7 |
| | polymerase integrated at yjhV and T7 |
| | promoters driving the expression of at least 2 |
| | phage genes selected from M13KO7 genes I-XI |

TABLE 3

| Gene | |
|---|---|
| (SEQ ID NO | |
| GenBank No. | |
| Name) Construct | Gene sequence with Regulatory Sequences |
| name | (T7 promoter and T7 Terminator) |
| SEQ ID NO: 15 | taatacgactcactatagggcgaattgggtaccgggcccagaccacaacggtttccctctagaaataatttt |
| NP_510893.1 | gtttaactttaagaaggagatatacatATGGCTGTTTATTTTGTAACAGGAAAACTAGGCTC |
| M13 genes I and XI | CGGCAAGACCTTAGTCAGCGTTGGTAAGATCCAGGATAAGATCGTGG |
| (upon integration | CCGGTTGTAAAATTGCCACCAACCTGGATCTTCGCCTGCAAAACCTG |
| into KTx00004 | CCACAGGTTGGCCGTTTTGCGAAAACCCCGCGTGTTCTGCGTATTCCG |
| termed | GATAAGCCGAGCATCAGCGACTTGCTGGCGATCGGTCGCGGCAACG |
| KTx00004.1.11) | ACTCGTACGACGAGAATAAAAACGGTCTGCTGGTGTTGGACGAGTGC |
| | GGTACATGGTTCAACACCCGTTCTTGGAATGATAAAGAAAGACAACC |
| | GATCATTGACTGGTTCCTGCACGCTCGCAAATTGGGTTGGGATATCA |
| | TCTTTCTGGTCCAGGACCTGTCAATCGTGGACAAACAAGCACGTTCC |
| | GCGCTGGCTGAACATGTGGTGTACTGCCGTCGTCTGGACCGTATCAC |
| | CCTGCCGTTCGTGGGCACCCTGTACTCTCTGATTACGGGTAGCAAGA |
| | TGCCGCTGCCGAAACTGCACGTCGGTGTGGTTAAGTATGGTGATAGC |
| | CAGCTTTCCCCGACCGTCGAGCGCTGGCTGTATACCGGTAAGAATCT |
| | TTACAATGCATATGATACGAAGCAAGCGTTTAGCTCTAACTATGACT |
| | CCGGTGTTTATTCGTACCTCACTCCGTATTTGAGCCATGGTCGTTATT |
| | TCAAACCGCTGAACTTGGGCCAGAAAATGAAATTGACCAAAATTTAT |
| | CTGAAAAAATTCAGCCGTGTTCTTTGCTTAGCGATCGGCTTCGCAAG |
| | CGCGTTTACGTACAGCTACATTACGCAGCCGAAACCCGAAGTTAAAA |
| | AAGTTGTAAGCCAGACCTACGACTTTGATAAGTTCACCATTGACTCC |
| | TCCCAACGTCTGAATCTGTCGTACCGCTACGTGTTTAAGGATAGTAA |
| | GGGCAAGCTCATCAACAGCGATGATTTACAGAAGCAAGGTTATAGCT |
| | TGACCTACATTGACTTGTGTACCGTTTCTATCAAAAAGGGAAACAGC |
| | AACGAGATTGTGAAGTGCAATTAAggatccggctgctaacaaagcccgaaaggaagctg |
| | agttggctgctgccaccgctgagcaataactagcataaccccttggggcctctaaacgggtcttgaggggtt |
| | ttttgACAAAGAGGGCGAGCGTTAA |
| SEQ ID NO: 16 | taatacgactcactatagggcgaattgggtaccgggcccagaccacaacggtttccctctagaaataatttt |
| NP_510885.1 | gtttaactttaagaaggagatatacatATGATAGATATGTTAGTATTGAGGCTACCCTTCAT |
| M13 genes II and 10 | CGACAGTCTTGTGTGCTCACGTTTGTCGGGAAACGATCTGATCGCGTT |
| KTx00004.2.10 | CGTGGACTTGTCTAAAATTGCTACTCTGTCTGGTATGAACCTGAGCGC |
| | GCGTACTGTAGAGTACCATATTGATGGTGACTTGACCGTTAGCGGCC |
| | TGTCCCACCCGTTTGAAAGCTTGCCGACCCATTATAGCGGCATCGCG |
| | TTCAAAATCTACGAAGGTTCCAAGAACTTTTACCCGTGTGTTGAGATT |
| | AAGGCGTCCCCCGCAAAAGTTCTGCAAGGTCACAACGTCTTTGGTAC |
| | GACCGATCTCGCCCTCTGTAGCGAAGCACTGCTGCTAAACTTTGCTA |
| | ATAGTTTACCGTGCCTGTATGACTTGCTGGACGTGAATGCAACCACC |
| | ATCAGCCGCATTGATGCGACGTTTAGCGCGCGCGCACCGAATGAAAA |
| | TATCGCGAAACAGGTTATCGACCACCTGCGTAACGTTAGCAACGGTC |
| | AGACCAAAAGCACCCGTTCTCAAAACTGGGAAAGCACTGTCACCTGG |
| | AATGAAACCTCCCGTCATCGTACCCTGGTCGCATATTTGAAGCACGT |
| | GGAGCTGCAACATCAGATTCAACAGTTGTCCTCTAAGCCGAGCGCTA |
| | AGATGACCAGCTATCAGAAGGAACAGTTGAAGGTGTTGAGCAATCC |
| | GGACCTGTTGGAATTTGCGAGTGGCCTGGTGCGTTTCGAGGCGCGCA |
| | TCAAAACCAGATACCTGAAGAGCTTTGGCCTGCCACTGAACCTGTTC |
| | GATGCGATTCGTTTTGCGTCTGATTACAACTCGCAAGGTAAAGATTT |
| | AATCTTTGATTTATGGAGCTTCTCCTTCAGCGAGCTATTCAAAGCCTT |
| | CGAGGGTGACTCTATGAATATCTATGATGATAGCGCGGTCCTGGACG |
| | CCATCCAAAGCAAACACTTTACGATTACCCCGAGCGGCAAGACCTCG |
| | TTCGCCAAGGCTAGCCGCTACTTCGGTTTTTACCGCCGTCTGGTTAAC |
| | GAGGGCTACGACTCAGTTGCACTGACCATGCCGCGTAACTCCTTCTG |
| | GCGTTACGTGTCCGCTCTGGTGGAGTGCGGCATTCCGAAAAGCCAGC |
| | TGATGAACCTGTCTACGTGCAACAACGTTGTTCCACTCGTGCGCTTCA |
| | TTAATGTGGACTTTTCCTCTCAGCGTCCGGACTGGTATAACGAGCCG |
| | GTTCTGAAAATCGCGTAAggatccggctgctaacaaagcccgaaaggaagctgagttggctgct |
| | gccaccgctgagcaataactagcataaccccttggggcctctaaacgggtcttgaggggttttttgACAAAG |
| | AGGGCGAGCGTTAA |
| SEQ ID NO: 17 | taatacgactcactatagggcgaattgggtaccgggcccagaccacaacggtttccctctagaaataatttt |
| NP_510891.1 | gtttaactttaagaaggagatatacatATGAAAAAGTTATTGTTTGCTATACCCCTAGTCGT |
| M13 gene III | TCCGTTTTATAGCCACAGCGCGGAAACCGTGGAATCTTGTCTGGCGA |
| KTx00004.3 | AACCGCATACGGAAAACTCTTTTACCAATGTGTGGAAAGATGATAAG |
| | ACCCTGGATAGATATGCGAACTATGAGGGTTGTCTTTGGAATGCGAC |
| | CGGCGTGGTGGTGTGCACCGGCGACGAAACCCAATGTTATGGTACTT |
| | GGGTTCCGATTGGTTTGGCGATCCCGGAGAACGAGGGCGGGGGCTCT |
| | GAGGGCGGCGGTAGCGAGGGTGGCGGTTCCGAGGGTGGAGGTACCA |
| | AACCGCCGGAATACGGTGACACCCCGATCCCGGGTTATACGTACATC |
| | AACCCGTTGGACGGCACGTACCCGCCAGGTACTGAGCAGAATCCGGC |
| | AAACCCAAATCCGAGCCTGGAAGAGAGCCAACCGTTGAATACCTTCA |
| | TGTTTCAGAACAACCGCTTCCGCAACCGTCAGGGTGCGCTGACCGTT |
| | TATACCGGTACAGTTACCCAGGGTACGGATCCGGTGAAAACCTACTA |
| | TCAGTACACTCCGGTCAGCTCGAAAGCCATGTACGATGCGTATTGGA |
| | ACGGTAAGTTCCGCGATTGCGCCTTCCACAGCGGCTTCAACGAAGAT |
| | CCGTTCGTGTGCGAATACCAAGGTCAGTCGAGCGACCTGCCGCAGCC |
| | GCCCGTTAATGCTGGTGGCGGGAGCGGTGGCGGTAGTGGTGGCGGC |
| | AGCGAAGGCGGTGGATCCGAAGGGGGGGGTTCCGAGGGCGGTGGCT |
| | CGGAGGGTGGTGGCTCTGGCGGTGGCTCTGGCTCCGGCGACTTCGAT |
| | TACGAAAAAATGGCGAACGCGAACAAGGGTGCTATGACCGAGAACG |
| | CGGATGAAAACGCTCTGCAGAGCGATGCAAAGGGTAAGCTGGACAG |
| | CGTTGCGACTGACTACGGCGCCGCAATTGACGGCTTCATCGGCGACG |
| | TTAGCGGTTTGGCTAACGGCAATGGTGCAACGGGTGACTTTGCGGGC |
| | TCCAATAGCCAAATGGCACAGGTTGGTGACGGTGACAACAGCCCGCT |
| | GATGAATAACTTTCGTCAATACCTCCCAAGCCTGCCGCAATCCGTGG |
| | AATGCCGTCCGTTCGTATTCTCCGCGGGTAAGCCTTATGAGTTCTCAA |
| | TCGACTGCGATAAGATTAACCTGTTTCGTGGTGTTTTTGCGTTTCTGC |
| | TGTACGTGGCCACCTTTATGTATGTCTTTAGCACCTTCGCCAACATTT |
| | TACGTAATAAAGAGAGCTAAggatccggctgctaacaaagcccgaaaggaagctgagttggc |
| | tgctgccaccgctgagcaataactagcataaccccttggggcctctaaacgggtcttgaggggttttttgAC |
| | AAAGAGGGCGAGCGTTAA |
| SEQ ID NO: 18 | taatacgactcactatagggcgaattgggtaccgggcccagaccacaacggtttccctctagaaataatttt |
| NP_510894.1 | gtttaactttaagaaggagatatacatATGAAACTATTAAATGTTATAAACTTTGTATTCCT |
| M13 gene IV | GATGTTTGTTTCGTCCTCATCTTTCGCCCAGGTTATTGAGATGAATAA |
| KTx00004.4 | CTCCCCGCTGCGTGATTTTGTCACTTGGTATAGCAAGCAGAGCGGCG |
| | AAAGCGTCATTGTTTCCCCGGATGTTAAAGGCACCGTTACCGTTTATA |
| | GCAGCGACGTTAAGCCGGAAAATCTGCGCAACTTCTTCATCAGCGTG |
| | CTACGGGCGAATAACTTCGATATGGTTGGTAGCATCCCGAGCATTAT |
| | TCAGAAATACAATCCGAATAACCAAGATTACATTGACGAACTGCCGA |
| | GCTCGGACAACCAAGAGTATGATGATAACAGCGCTCCGTCCGGTGGT |
| | TTTTTTGTACCGCAGAATGATAACGTAACGCAGACGTTTAAAATCAA |
| | CAATGTGCGCGCGAAAGATCTGATCCGTGTCGTGGAACTGTTCGTGA |
| | AGTCTAACACGAGCAAGAGCTCTAACGTGTTGTCTATCGACGGCTCC |
| | AACTTATTAGTTGTTTCCGCGCCAAAAGACATCTTGGACAACCTGCCT |
| | CAGTTTCTGTCCACCGTGGACCTGCCGACCGACCAGATTCTGATTGA |
| | GGGCTTGATTTTTGAAGTTCAGCAAGGTGACGCATTGGACTTCAGCT |
| | TCGCGGCGGGCAGCCAACGCGGTACAGTTGCTGGTGGCGTGAACACC |
| | GATCGTTTGACCAGCGTTCTCTCTAGCGCGGGGGGTTCATTCGGCATC |
| | TTCAACGGTGATGTCTTGGGTCTGTCGGTTCGTGCCCTGAAAACCAAT |
| | TCTCACAGCAAGATCCTGTCCGTGCCGCGTATTCTGACCCTGTCAGG |
| | ACAAAAAGGTTCCATCTCCGTGGGCCAAAACGTCCCATTTATCACGG |
| | GTCGTGTGACCGGCGAGAGCGCGAACGTTAACAACCCGTTCCAGACC |
| | ATCGAGCGCCAGAACGTGGGTATTAGCATGTCCGTGTTCCCGGTAGC |
| | AATGGCAGGCGGTAATATTGTGCTGGACATCACGAGTAAGGCGGATT |
| | CGCTGAGCTCTTCGACTCAGGCAAGCGACGTCATCACCAACCAAAGA |
| | AGCATCGCCACCACCGTTAATCTGCGTGATGGTCAAACCCTGCTGCT |
| | GGGCGGTTTGACTGACTACAAAAATACCTCGCAAGACAGCGGCGTGC |
| | CGTTTTTGTCAAAGATCCCGCTGATAGGCCTGTTGTTTAGCAGCCGTT |
| | CTGACAGTAATGAAGAGAGCACTCTTTACGTGCTGGTTAAGGCTACC |
| | ATTggatccggctgctaacaaagcccgaaaggaagctgagttggctgctgccaccgctgagcaataactagc |
| | ataaccccttggggcctctaaacgggtcttgaggggttttttgACAAAGAGGGCGAGCGTTAA |
| SEQ ID NO: 19 | taatacgactcactatagggcgaattgggtaccgggcccagaccacaacggtttccctctagaaataatttt |
| NP_510887.1 | gtttaactttaagaaggagatatacatATGATTAAAGTAGAAATAAAGCCCTCACAAGCGC |
| M13 gene V | AGTTCACGACCCGTTCCGGTGTTAGCCGTCAGGGTAAGCCGTATAGC |
| KTx00004.5 | TTAAATGAGCAGCTGTGCTACGTGGACCTGGGCAACGAGTACCCGGT |
| | TTTGGTCAAAATCACCCTGGACGAAGGCCAACCGGCTTACGCACCGG |
| | GTCTGTATACCGTGCACCTGAGCTCTTTTAAAGTTGGTCAATTCGGCT |
| | CGCTGATGATTGATCGTTTGCGCCTCGTGCCAGCGAAGTAAggatccggct |
| | gctaacaaagcccgaaaggaagctgagttggctgctgccaccgctgagcaataactagcataaccccttggg |
| | gcctctaaacgggtcttgaggggttttttgACAAAGAGGGCGAGCGTTAA |
| SEQ ID NO: 20 | taatacgactcactatagggcgaattgggtaccgggcccagaccacaacggtttccctctagaaataatttt |
| NP_510892.1 | gtttaactttaagaaggagatatacatATGCCAGTACTGTTAGGAATACCCCTATTGTTACG |
| M13 gene VI | TTTTTTGGGTTTCTTGCTGGTCACCCTGTTCGGCTACTTGCTCACGTTT |
| KTx00004.6 | CTGAAAAAAGGTTTTGGCAAGATCGCGATCGCCATCAGCCTGTTCCT |
| | GGCACTGATTATCGGCCTGAATAGCATTCTGGTTGGTTACCTGAGCG |
| | ACATCTCTGCGCAGCTGCCGTCGGACTTCGTGCAAGGTGTTCAACTG |
| | ATTCTGCCGTCCAACGCTTTGCCGTGCTTCTATGTTATTTTGTCCGTG |
| | AAGGCGGCGATTTTCATCTTTGATGTAAAGCAGAAGATCGTGAGCTA |
| | TCTTGACTGGGATAAATAAggatccggctgctaacaaagcccgaaaggaagctgagttggctg |
| | ctgccaccgctgagcaataactagcataaccccttggggcctctaaacgggtcttgaggggttttttgACAAA |
| | GAGGGCGAGCGTTAA |
| SEQ ID NO: 21 | taatacgactcactatagggcgaattgggtaccgggcccagaccacaacggtttccctctagaaataatttt |
| NP_510888.1 | gtttaactttaagaaggagatatacatATGGAACAAGTAGCTGATTTTGACACAATATACC |
| M13 gene VII | AGGCAATGATTCAGATTAGCGTTGTGTTGTGCTTCGCTCTGGGCATCA |
| KTx00004.7 | TCGCGGGTGGTCAACGTTAAggatccggctgctaacaaagcccgaaaggaagctgagttggct |
| | gctgccaccgctgagcaataactagcataaccccttggggcctctaaacgggtcttgaggggttttttgACA |
| | AAGAGGGCGAGCGTTAA |
| SEQ ID NO: 22 | taatacgactcactatagggcgaattgggtaccgggcccagaccacaacggtttccctctagaaataatttt |
| NP_510890.1 | gtttaactttaagaaggagatatacatATGAAAAAGTCACTAGTATTAAAAGCTAGTGTCG |
| M13 gene VIII | CTGTGGCCACGTTGGTTCCGATGCTGTCGTTCGCCGCGGAAGGTGAT |
| KTx00004.8 | GACCCGGCAAAAGCTGCGTTTAACAGCCTGCAGGCGTCTGCTACCGA |
| | GTACATCGGCTATGCGTGGGCAATGGTGGTGGTTATTGTTGGTGCGA |
| | CCATTGGCATCAAGCTGTTTAAAAAGTTCACCAGCAAGGCGTCCTAA |
| | ggatccggctgctaacaaagcccgaaaggaagctgagttggctgctgccaccgctgagcaataactagcata |
| | accccttggggcctctaaacgggtcttgaggggttttttgACAAAGAGGGCGAGCGTTAA |
| SEQ ID NO: 23 | taatacgactcactatagggcgaattgggtaccgggcccagaccacaacggtttccctctagaaataatttt |
| NP_510889.1 | gtttaactttaagaaggagatatacatATGAGTGTACTAGTTTATTCATTTGCTTCTTTCGTG |
| M13 gene IX | CTGGGTTGGTGCTTGCGCTCCGGCATCACCTACTTTACCCGTCTGATG |
| KTx00004.9 | GAAACCAGCAGCTAAggatccggctgctaacaaagcccgaaaggaagctgagttggctgctgcca |
| | ccgctgagcaataactagcataaccccttggggcctctaaacgggtcttgaggggttttttgACAAAGAGG |
| | GCGAGCGTTAA |

### Example 4: Making Production Host Cell with Decreased p5 Activity

The production strain KTx00003 is further developed to decrease the relative production of p5 (KTx00006). p5 is a ssDNA binding protein. Changing 5 bases in the 5′ UTR of gene V inhibits the interaction of the gene V mRNA with the ribosome and reduces the amount of p5 protein that is translated by the ribosome.

The replicative, dsDNA form of the phagemid is used as a template to produce cssDNA through rolling circle amplification. After the cssDNA is produced inside the bacterial host, two different things can happen to it. If p5 levels are low, p5 does not bind to the cssDNA thus allowing the bacterial replication machinery to polymerize a complementary strand and create more replicative dsDNA. This replicative dsDNA will in turn be used as additional rolling circle amplification template leading to the creation of more cssDNA. If, however, the p5 levels are high, then p5 will bind the cssDNA and prevent the bacterial host machinery from polymerizing the complementary strand thus preventing it from being used as rolling circle amplification template. P5 binding to the cssDNA will instead cause the cssDNA to be packaged into the phage capsid and exported out of the host cell. Reducing translation of p5 allows the cell to produce more phage cssDNA template before switching to packaging the cssDNA into the phage capsid and exporting the phage capsid out of the cell. The accumulation of additional cssDNA template within the E. coli cell leads to increased production of packaged cssDNA. A schematic of this strategy is shown in FIG. 6 (see Lee et al., Optimizing protein V untranslated region sequence in M13 phage for increased production of single-stranded DNA for origami. Nucleic Acids Res. 2021 Jun. 21; 49(11):6596-6603, which is herein incorporated by reference in its entirety)

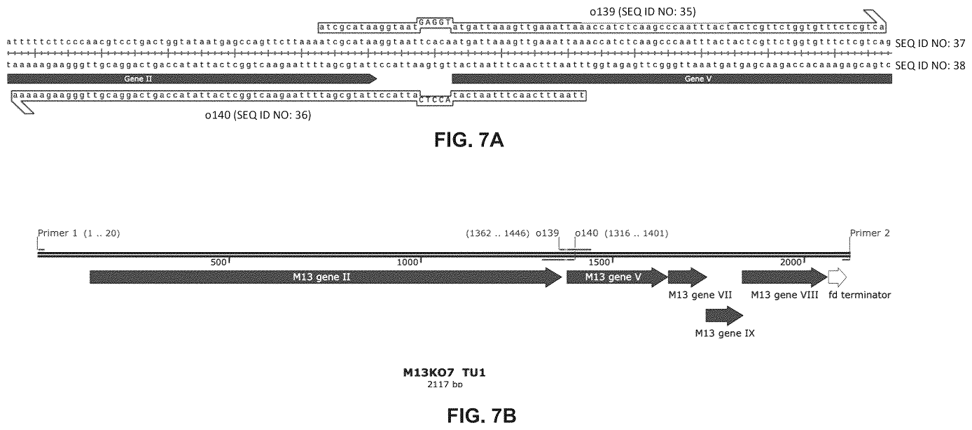

Prior to the cloning of the first transcriptional unit described in Example 1, above, PCR primers are designed to amplify the first transcriptional unit such that the 5′ UTR (untranslated region) of gene V is changed from the wild-type TCACA to GAGGT (as shown in FIG. 7, panel A).

PCR primers are also designed to amplify the rest of Transcription Unit 1 described in Example 1 through fusion PCR, such that the mutated 5′ UTR of gene V is incorporated into the Transcription Unit 1 creating a 2117 bp variation of Transcription unit 1 including the altered gene V 5′ UTR sequence (as shown in FIG. 7, panel B).

Identical E. coli production host strains are created with the two alternative versions of Transcription Unit 1; one containing the mutation in the 5′ UTR and the other lacking the mutation. Both strains are transformed with the same phagemid and cultured side by side so that cssDNA production can be compared between the two. Without wishing to be bound by any theory, it is believed that the KTx00006 strain with the mutation in the 5′ UTR of gene V will produce more cssDNA than the control strain (KTx00003) lacking the mutation. Analysis of the cssDNA produced from the strains can be performed as previously described.

### Example 5: Creating a Circular Single-Stranded DNA Phagemid with Plasmid Ori

The minimal phagemid backbone is cloned from the M13KO7 template by amplifying the M13 origin of replication including the packaging signal with primers that contain 30 nt homology tails to the other fragments. The pyrF gene is amplified from the E. coli MG1655 genome and synthetic promoter and terminator sequences are attached using primer tails. The pUC19 origin of replication is amplified from pUC19 (Catalog number N3041S, New England Biolabs, Ipswich, MA, USA) with primer tails that are homologous to the other fragments. These three fragments are assembled via Gibson isothermal assembly Gibson et al., Nat Methods. 2009 May; 6(5):343-5. Golden Gate Assembly sites (Engler et al., PLoS One. 2008; 3(11):e3647) are inserted into this phagemid through their inclusion in the primer tails that were used to amplify and assemble the fragments. The Golden Gate Assembly sites include BsaI, BsmBI and PaqCI. The M13 origin of replication is included in the design so that the phagemid can replicate as cssDNA in E. coli. The packaging signal is included so that the M13 proteins will package the cssDNA into the phage particle. The pUC19 origin of replication is included so that the phagemid can replicate as dsDNA in E. coli. The pyrF gene is used as a selectable auxotrophic marker for use in the KTx00001 and its descendants which are uracil auxotrophs due to the disruption of the genomic copy of the pyrF gene via the insertion of M13KO7 transcription unit 1 (see example 1). The Golden Gate Assembly sites are included to make inserting user defined sequences easy. User defined sequences can be synthesized or amplified with primers such that they are flanked by BsaI, BsmBI or PaqCI restriction sites. The insert and the minimal phagemid are then incubated together with the appropriate restriction enzyme and buffer and ligase and the temperature is cycled between 37 C for restriction enzyme cutting and 16 C for ligation. Because the restriction enzyme recognition sequences are asymmetrical and are a few bases away from the cut site, the recognition sequences are inserted into the phagemid and the inserts in such a way that they are cleaved off after the initial digestion. If the parts then assemble in the desired manner, inserting the user defined sequence into the phagemid, then the recognition sequence is ablated and no more cutting can ensue. If however, the fragments re-ligate to the recognition sequence reforming the initial inputs, the recognition sequence is recreated and another round of cutting can ensue. In this way the reaction proceeds unidirectionally until almost all restriction recognition sites have been abolished and almost all of the inserts have been correctly inserted into the phagemid backbone.

Notably, the pyrF auxotrophic marker is included in the phagemid in place of traditional antibiotic markers to increase the safety of the cssDNA produced from the system. Since one of the applications of the cssDNA produced from this system is human therapeutic gene and cell therapy, it is not desirable to include antibiotic marker sequences. The exclusion of antibiotic marker sequences prevents this system from spreading antibiotic resistance to microbes that may infect human patients with antibiotic resistant strains.

### Example 6: Increased cssDNA Yield with Increased Phagemid Copy Number

Given the interplay between multiple factors including both helper plasmid copy number (or copy numbers of integrated phage genes), phage gene expression, and phagemid copy number for fermentation, it was unpredictable how varying phagemid copy number would affect cssDNA yield in a fermentation-based production method. This example provides results demonstrating that increasing phagemid copy number increased cssDNA production from a bacterial production strain containing the phagemid and a helper plasmid.

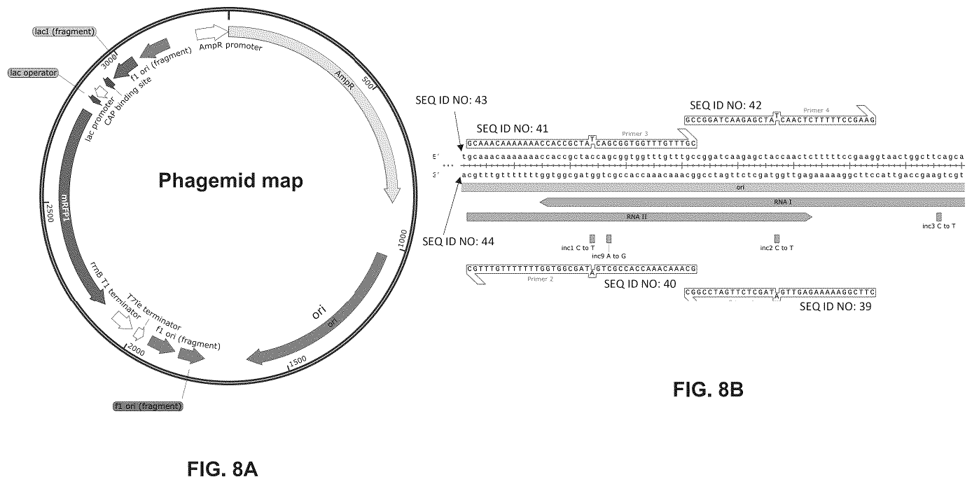

A series of phagemids containing different origins of replication was constructed as shown in Table E1 below. A map of the phagemid containing the pUC19 origin is shown in FIG. 8A, and the primers used to generate the inc1 and inc2 mutations in the pUC19 origin are shown in FIG. 8B. The phagemids were tested in combination with two different helper plasmids: KHP0 and KHP1, which each contain the p15A origin of replication. KHP0 was derived from helper phage M13KO7 by removing the F1 origin and the packaging signal. This was accomplished by amplifying the kanamycin marker and all of the protein coding genes from M13KO7 with primers designed to exclude the F1 origin of replication and the packaging signal and then re-circularizing the amplified DNA using a DNA ligase in vitro reaction. KHP1 was similarly derived from helper phage M13KO7, but further comprises gene V attenuation, which was created by mutating 5 base pairs immediately upstream of gene V causing reduced translation rates for gene V. Gene V is involved in regulating the transition from replicating double-stranded DNA into non-replicating single-stranded DNA within the bacterial cells. Decreasing expression of gene V has been shown to result in a longer phase of replicating as double-stranded DNA and therefore greater accumulation of dsDNA template resulting in higher cssDNA yield.

TABLE E1
Phagemids with varying copy numbers

| Phagemid number | Description |
|---|---|
| 111 | Phagemid with standard pUC19 |
| | origin (referred to as “wt”) |
| 120 | Phagemid with p15A origin (reduced |
| | copy number; referred to as “red”) |
| 161 | Phagemid with inc2 mutated pUC19 |
| | origin (“inc2”) |
| 162 | Phagemid with incl mutated pUC19 |
| | origin (“inc1”) |
| 163 | Phagemid with inc1 and ine2 |
| | mutations in pUC19 origin (referred |
| | to as “inc1&2”) |

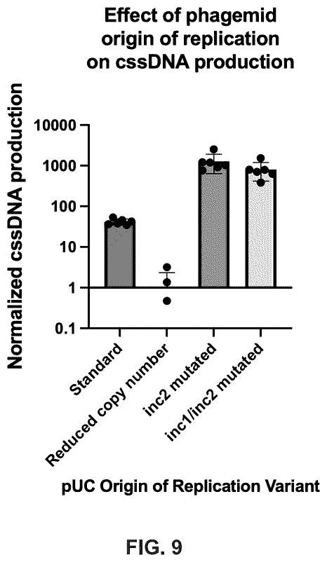

Standard NEB5alpa chemically competent E. coli cells were co-transformed with Kano Helper Plasmid 1 (KHP1) and one of 4 phagemid variants as shown in FIG. 9. Individual colonies were selected on LB agar plates supplemented with 50 ug/ml kanamycin (helper plasmid) and 100 ug/ml carbenicillin (phagemid). Six individual colonies were picked from each plate and inoculated into starter cultures of 2xYT medium supplemented with 50 ug/ml kanamycin and 100 ug/ml carbenicillin. The starter cultures were grown overnight to saturation and then diluted to approximately OD600 0.04 in the same medium. Cultures were then grown at 30 degrees C. with shaking for 18 hours. The cultures were harvested and the medium clarified of bacterial cells through centrifugation. The supernatant containing the phage particles and therefore the cssDNA was pipetted into fresh vessels and the concentration of the cssDNA in each sample was analyzed by qPCR using primers and probes that target the F1 origin of replication on the cssDNA and a FAM reporter and a NFQ-MGB quencher.

As shown in FIG. 9, inc2 and inc1&2 phagemid mutations resulted in higher cssDNA yields relative to the “standard” pUC19 phagemid. The reduced copy number phagemid resulted in significantly lower yield compared to standard pUC19, inc2, or inc1&2. The inc1 mutation is a C59T mutation relative to a standard pUC origin, wherein the nucleotide numbering is based on the sequence of the standard pUC origin shown in SEQ ID NO: 27. The inc2 mutation is a C92T mutation relative to a standard pUC origin, wherein the nucleotide numbering is based on the sequence of the standard pUC origin shown in SEQ ID NO: 27. The inc2 and inc1/inc2 phagemid used in these experiments further included the mutation G62A relative to the standard pUC origin shown in SEQ ID NO: 27, but this mutation is not expected to affect function of the origin.

These results suggest that increased phagemid copy number can result in increased cssDNA yield produced from the phagemid. The reduced copy number origin results in an estimated 15 copies per cell, while the standard pUC19 origin provides an estimated 1000 copies per cell, and the inc1&2 mutated pUC19 origin provides an estimated 4000 copies per cell. These copy number estimates vary greatly depending on the growth medium and the growth stage.

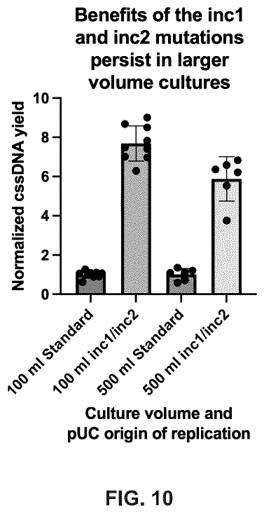

A significantly increased cssDNA yield with a phagemid containing inc1&2 mutations was also confirmed in larger scale cultures. Standard NEB5alpa chemically competent E. coli cells were co-transformed with Kano Helper Plasmid 1 (KHP1) and one of 2 phagemid variants (standard (pUC19) or inc1/inc2) as shown in FIG. 10. Individual colonies were selected on LB agar plates supplemented with 50 ug/ml kanamycin (helper plasmid) and 100 ug/ml carbenicillin (phagemid). 15 individual colonies were picked from each plate and inoculated into starter cultures of 2xYT medium supplemented with 50 ug/ml kanamycin and 100 ug/ml carbenicillin. The starter cultures were grown overnight to saturation and then diluted to approximately OD600 0.04 in the same medium in either 100 ml (9 cultures) or 500 ml (6 cultures) volumes. Cultures were then grown at 30 degrees C. with shaking for 18 hours. The cultures were harvested and the medium clarified of bacterial cells through centrifugation. The supernatant containing the phage particles and therefore the cssDNA was pipetted into fresh vessels and the concentration of the cssDNA in each sample was analyzed by qPCR using primers and probes that target the F1 origin of replication on the cssDNA and a FAM reporter and a NFQ-MGB quencher. As shown in FIG. 10, the inc1&2 mutations in the phagemid resulted in at least a 5-fold increase in cssDNA yield compared to the yield obtained with a wild-type pUC19 origin. The increased cssDNA yield was reproducible for both 100 mL culture production and 500 mL culture production.

The above results demonstrate that inc mutations in a pUC19 phagemid origin of replication for cssDNA production confer a significant cssDNA yield increase relative to the pUC19 origin of replication (e.g., about a 5- to 10-fold increased yield was observed relative to a wild-type pUC19 origin, and more than a 100-fold increased yield relative to a p15A origin).

### Example 7: Increased cssDNA Yield with Increased Helper Plasmid Copy Number

This example provides results demonstrating that increased helper plasmid copy number can also increase cssDNA yield, including in combination with a high-copy number phagemid (inc1&2 phagemid, with an estimated 4000 copies per cell).

A series of different helper plasmids was constructed as shown in Table E2 below.

TABLE E2
Helper plasmids with varying copy numbers

| Helper plasmid number | Description |
|---|---|
| 81 | Helper plasmid with pSC101 ori |
| | (very low copy number-estimated 5 copies |
| | per cell) |
| 114 | Helper plasmid with p15A ori |
| | (Low copy number-estimated 15 copies per |
| | cell) |
| 148 | Helper plasmid with pUC19 ori (High |
| | copy-estimated 1000 copies per cell) |

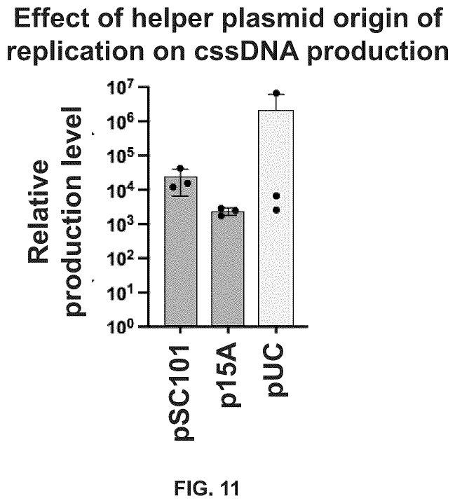

E. coli production host cells were transformed with the indicated helper plasmid from Table E2 in combination with the phagemid 163 described in Example 6. Individual colonies of double-transformed E. coli were picked and grown in in a starter culture, which was then diluted and grown for small-scale production analysis. After 18 hours culture, the bacteria were lysed and relative cssDNA yield was analyzed by qPCR. As shown in FIG. 11, the pUC19 origin helper plasmid (148) resulted in increased yield of cssDNA at 18 hours from the phagemid than the lower copy number helper plasmids 81 (pSC101 ori), and 114 (p15A ori).

### Example 8: Creation of a Production Strain

### Selection of the Host Strain

The first step was to choose to the background strain in which to engineer the production machinery. Production of cssDNA has typically been carried out in E. coli cloning host strains such as DH5a, JM109, XL-1 Blue, or M1061. These common lab strains have several advantages as they have been engineered to optimize plasmid DNA cloning and preparation. They have recA deleted to reduce recombination events in the desired cssDNA product. They have endA deleted to reduce DNA degradation. They have host nucleases deleted to increase transformation efficiency. They have the F pilus evicted to avoid reinfection of cells by infectious phage particles. They are also legacy strains however, that were domesticated years ago and engineered or mutated with other deletions that may or may not be beneficial for cssDNA production. They generally have slower growth rates and lower biomass yield than wild type strains without as many mutations.

We hypothesized that a wild-type strain may serve as a better production host due its generally improved fitness compared with the heavily engineered lab strains including improved growth rate and biomass yield per unit of medium provided. To test this hypothesis, we acquired a small library of E. coli background strains including the common lab strains mentioned above and others that are less domesticated. These included BW25113, K12, and MG1655. The full list of strains is included in Table 4.

TABLE 4
E. coli strains screened for growth in defined medium

| Strain | Back- | Catalogue | |
|---|---|---|---|
| ID | ground | Number | Genotype |
| bac001 | DH5α | NEB | endA1 glnV44 thi-1 recA1 relA1 |
| | | C2987U | gyrA96 deoR nupG purB20 |
| | | | φ80dlacZAM15 Δ(lacZYA- |
| | | | argF)U169, hsdR17(rK−mK+), λ- |
| bac002 | K12 | DSMZ 498 | Wild type |
| bac003 | MG1655 | DSMZ 18039 | λ-ilvG-rfb-50 rph-1 |
| bac004 | HMS174 | DSMZ 5932 | recA1, hsdR(rK12-mK12+) |
| | (DE3) | | (RifR) |
| bac005 | JM109 | DSMZ 3423 | recA1 endAl gyr96A thi hsdR17 |
| | | | supE44 relA1 lambda- |
| | | | Delta(lac-proAB) (F′ traD36 |
| | | | proAB laqIqZDeltaM15) |
| bac006 | BL21 | Sigma | ompT gal dcm lon hsdSB(rB-mB-) |
| | | 69449-3 | [malB+]K−12(λS) |
| bac007 | BL21 | Biosearch | ompT gal dcm lon hsdSB(rB-mB-) |
| | (DE3) | 60810-1 | λ(DE3 [lacI lacUV5- |
| | | | T7p07 ind1 sam7 nin5]) |
| | | | [malB+]K−12(λS) |
| bac008 | XL1-Blue | Agilent | Δ(mcrA)183 Δ(mcrCB-hsdSMR- |
| | MRF | 200158 | mrr)173 endA1 supE44 thi-1 |
| | | | recA1 gyrA96 relAl lac [F proAB |
| | | | lacIqZΔM15 Tn10 (Tetr)] |
| bac009 | XL1-Blue | Agilent | recA1 endA1 gyrA96 thi-1 hsdR17 |
| | | 200249 | supE44 relA1 lac [F proAB |
| | | | lacIqZΔM15 Tn 10 (Tetr)] |
| bac010 | MC1061F- | Biosearch | araD139 Δ(ara-leu)7696 galE15 |
| | | 60514-1 | galK16 Δ(lac)X74 rpsL (StrR) |
| | | | hsdR2 (rK−mK+) mcrA mcrB1 |
| bac011 | DH5a | Thermofisher | Φ80lacZΔM15 Δ(lacZYA-argF) |
| | | 18258012 | U169 recA1 endA1 bsdR17 |
| | | | (rk−, mk+) phoA supE44 λ-thi-1 |
| | | | gyrA96 relAl |
| bac012 | BW25113 | Keio | lacI+rrnBT14 ΔlacZWJ16 |
| | | JW1273-1 | hsdR514 ΔaraBADAH33 |
| | | | ΔrhaBADLD78 rph-1 |
| | | | Δ(araB−D)567 Δ(rhaD−B)568 |
| | | | ΔlacZ4787(::rmB-3) Kan::pyrF” |
| bac013 | BW25113 | Keio | lacI+rmBT14 ΔlacZWJ16 |
| | | JW1273-2 | hsdR514 ΔaraBADAH33 |
| | | | ΔrhaBADLD78 rph-1 |
| | | | Δ(araB−D)567 Δ(rhaD−B)568 |
| | | | ΔlacZ4787(::rmnB-3) Kan::pyrF |
| bac014 | DH5α | Thermofisher | Φ80lacZΔM15 Δ(lacZYA-argF) |
| | | 11319019 | U169 recA1 endA1 hsdR17 |
| | | | (rk−, mk+) phoA supE44 λ-thi-1 |
| | | | gyrA96 relA1 |
| bac015 | BL21 | NEB C2528J | fhuA2 [lon] ompT gal (λ DE3) |
| | (DE3) | | [dcm] ΔhsdS/pLemo(CamR) λ |
| | | | DE3 = λ sBamHIo ΔEcoRI-B |
| | | | int::(lacI::PlacUV5::T7 gene1) |
| | | | i21 Δnin5 pLemo = |
| | | | pACYC184-PrhaBAD-lysY |
| bac016 | BW25113 | Kano | lacI+rmBT14 ΔlacZWJ16 |
| | | bac012 | hsdR514 ΔaraBADAH33 |
| | | ΔKan | ΔrhaBADLD78 rph-1 |
| | | | Δ(araB−D)567 Δ(rhaD−B)568 |
| | | | ΔlacZ4787(::rmB-3) ΔKan: pyrF |
| bac019 | MC1061F | Biosearch | F′[proAB lacIqZ ΔM15 Th10 |
| | | | (TetR)] araD139 Δ(ara-leu)7696 |
| | | 60512-1 | galE15 galK16 Δ(lac)X74 rpsL |
| | | | (StrR) hsdR2 (rK− mK+) |
| | | | mcrA merB1 |
| bac020 | BW25113 | Horizon | lacI+rmBT14 ΔlacZWJ16 |
| | | OEC5042 | hsdR514 ΔaraBADAH33 |
| | | | ΔrhaBADLD78 rph-1 |
| | | | Δ(araB−D)567 Δ(rhaD−B)568 |
| | | | ΔlacZ4787(::rmB-3)” |
| bac021 | BW25113 | Keio JW5807 | lacI+rrnBT14 ΔlacZWJ16 |
| | | | hsdR514 ΔaraBADAH33 |
| | | | ΔrhaBADLD78 rph-1 |
| | | | Δ(araB−D)567 Δ(rhaD−B)568 |
| | | | ΔlacZ4787(::rmB-3) ΔKan::leuB” |

We preferred to use a defined medium for production of cssDNA because this reduces the cost of scaled production, increases the reproducibility of production runs and allows for the use of auxotrophic markers rather than antibiotic markers thus increasing the safety of the final cssDNA product. We chose to use a defined medium recipe based on Riesenberg's 1991 recipe because it is well established and has been commonly used since its publication. Riesenberg, et al. High cell density cultivation of Escherichia coli at controlled specific growth rate. J Biotechnol. 1991 August; 20(1):17-27. The Reisenberg recipe was also used to successfully produce large amounts of cssDNA in a fed batch reactor system. Kick, et al. Efficient Production of Single-Stranded Phage DNA as Scaffolds for DNA Origami. Nano Lett. 2015 Jul. 8; 15(7):4672-6. We will hereafter refer to this medium as Riesenberg medium. However, other bacterial cell mediums could also be used.

We first screened our strain collection for growth in Riesenberg medium. Single colonies were inoculated in triplicate in LB medium and grown overnight as a preculture before being diluted to OD500 0.05 in the morning in Riesenberg medium. These cultures were incubated with shaking at 30° C. for 24 hours and the terminal OD600 was recorded. Cells from each strain were streaked on LB agar without antibiotics to obtain single colonies.

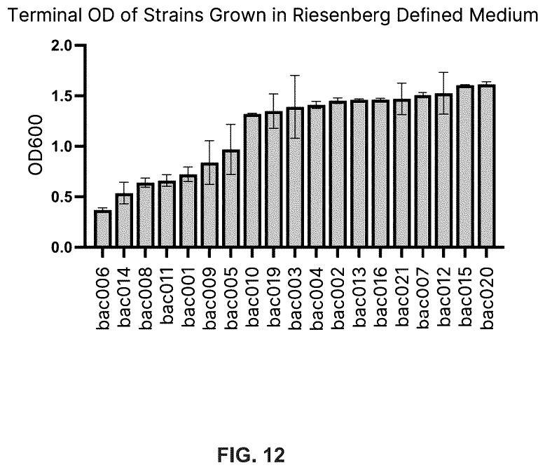

FIG. 12 shows the results comparing terminal OD for the tested strains. We indeed found that various isolates of DH5α, XL-1 Blue, MC1061 reached the lowest terminal OD, as shown in FIG. 12. Less engineered strains such as MG1655 and K12 reached a higher OD (FIG. 12). Various isolates of BW25113 reached the highest ODs (FIG. 12). These strains have slightly reduced genomes with deletions of the lactose, arabinose and rhamnose operons which may provide growth benefits since the cells need to replicate less DNA and produce less protein. These alternate sugar operons are not needed in a fermentation culture where glucose is provided as sole carbon source. Furthermore, since BW25113 was the parent strain of the Keio knockout collection, there already existed single-gene deletion strains in this background of genes essential for the synthesis of certain amino-acid and nucleosides. Indeed, three of these deletion strains, bac012 (ΔpyrF), bac013 (ΔpyrF), bac016 (ΔpyrF, Δkan) bac021 (ΔleuB) were included in our screen and all performed near the top. The best performing strain was the BW25113 parent of the single-gene deletion strains mentioned above, but the strains with either pyrF or leuB deleted were not far behind and conveniently they were already suitable for use in selecting for and maintaining plasmids with auxotrophic markers. Thus, we chose bac016 to move forward with due to its robust growth in our preferred medium and due to its being furthest along the engineering process already having deletions of pyrF and the kanamycin resistance marker. Other strains could also be used for cssDNA production according to any of the methods disclosed herein.

Having identified bac016 as a strain which grew well in our chosen defined medium, we next verified that it was able to produce cssDNA using a helper plasmid and phagemid prior to undertaking any genomic engineering steps. bac016 was co-transformed with a helper plasmid (cdsDNA114) and phagemid (cdsDNA111) system alongside the standard DH5α production host bac001. In another experiment, bac016 was co-transformed with the cdsDNA114 helper plasmid and the new cdsDNA117 phagemid expressing the pyrF auxotrophic marker instead of an antibiotic resistance gene.

The cdsDNA114 helper plasmid expresses all of the M13 genes and a kanamycin marker and carries a p15A origin of replication. It lacks the M13 origin of replication and packaging signal and therefore cannot replicate as an infectious phage particle. The cdsDNA111 phagemid has a pUC origin of replication and expresses RFP and an ampicillin resistance marker. Unlike the helper plasmid, it does contain the M13 origin of replication and packaging signal allowing it to be produced as cssDNA and packaged into the phage capsids. These capsids are incapable of replicating inside of a new bacterial host since they lack the necessary M13 genes. The cdsDNA117 phagemid is identical to the cds111 phagemid except that it expresses a pyrF auxotrophic marker rather than an ampicillin antibiotic marker.

Colonies from each of the three transformation reactions described above were inoculated in triplicate in Riesenberg medium and grown overnight at 37 C as pre-cultures with appropriate antibiotics and supplementation of uracil to complement the pyrF auxotrophy where necessary. Cultures were then diluted to OD600 0.05 and incubated 48 hours at 30 C to test production of cssDNA. Broth was harvested and centrifuged to separate E. coli cells from the supernatant which contains the secreted phage particles. The supernatant was then transferred to a clean tube and heated at 99 C for 10 minutes to lyse the phage particles that contain the cssDNA. This material was then used a template for qPCR targeting the F1 origin of replication that is present on the cssDNA.

This experiment showed that BW25113 could produce cssDNA when transformed with the traditional two-plasmid helper plasmid-phagemid production machinery. When transformed with the ampicillin resistant phagemid, it produced the same amount of cssDNA as the traditional DH5α production host transformed with the same phagemid. When transformed with the new cdsDNA117 phagemid expressing the pyrF auxotrophic marker, it produced significantly more cssDNA.

Because DH5α lacks a deletion of pyrF, cssDNA production with a phagemid expressing the pyrF auxotrophic marker was not tested in this strain. However, strains such as DH5α could be engineered to have a deletion in pyrF for compatibility with auxotrophic selection.

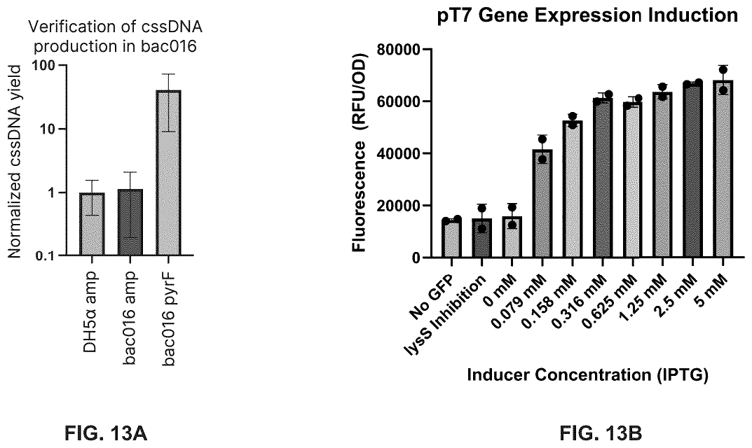

FIG. 13A shows the verification of cssDNA production in the unengineered BW25113 parent strain. The traditional production host, bac001 (DH5α) and the new proposed production host, bac016 (BW25113 ΔpyrF, Δkan) were each transformed with the traditional production machinery consisting of the cdsDNA114 helper plasmid and the cdsDNA111 phagemid. bac016 was additionally co-transformed with the same cdsDNA114 helper plasmid and the new cdsDNA117 phagemid expressing the pyrF auxotrophic marker. Colonies were inoculated in triplicate in Riesenberg medium with appropriate antibiotics and uracil supplementation to select for the plasmids and grown overnight in precultures before being diluted to OD600 0.05 in the same medium and incubated for another 48 hours at 30° C. to produce cssDNA. cssDNA was quantitated using qPCR targeting the F1 origin of replication that is present on the cssDNA backbone. The data confirm that the un-engineered host, bac016 produced cssDNA in equal amounts to the standard bac001 DH5α production host and in fact produced much more cssDNA when the cdsDNA117 phagemid carrying the pyrF auxotrophic marker was used as the cssDNA template.

### Integration of M13 Transcriptional Units

The M13 genome is organized in two separate transcriptional units. The first consists of genes II, X, V, VII, IX and VIII and is expressed from strong promoters. The second consists of genes III, VI, I, XI and IV and is expressed from weak promoters. Two of the genes overlap with the open reading frames of two other genes (i.e. the gene for p10 is completely contained within the coding region for p2 and the gene coding for p11 is completely contained withing the open reading frame for pI.) PA, PB, PH are strongly active promoters (see FIG. 1B). Two additional weak promoters are labeled PZ and PW. Two terminators (T) are strong, rho-independent terminators; the third, weaker terminator (T(weak)) is rho-dependent. Smeal et al., Simulation of the M13 life cycle I: Assembly of a genetically-structured deterministic chemical kinetic simulation, Virology, Volume 500, 2017, Pages 259-274.

In order to create a stable cell line capable of producing M13 phage particles while also being highly unlikely to recombine with a phagemid to produce a replication-competent phage, each transcriptional unit of the M13 bacteriophage containing protein coding genes and lacking a phage origin of replication and phage packaging signal was cloned from the publicly available helper phage M13KO7 (catalogue #N0315S, NEB, Ipswich, MA) (SEQ ID NO: 24) and integrated into a ΔpyrF version of the E. coli BW25113 genome produced as part of the Keio knockout collection called isolate JW1273-1 at two loci. Baba, et al, Construction of Escherichia coli K-12 in-frame, single-gene knockout mutants: the Keio collection. Mol Syst Biol. 2006; 2:2006.0008. In order to modulate M13 protein production, the gene coding for the T7 bacteriophage RNA-polymerase under control of the E. coli lac operator and promoter was also integrated into the host strain at another locus. This allows for inducible expression of the T7 polymerase by addition of Isopropyl β-d-1-thiogalactopyranoside (IPTG) and thereby inducible expression of the M13 phage genes. The E. coli BW25113 ΔpyrF Keio isolate JW1273-1 (bac012) host strain initially contained a spectinomycin resistance marker flanked by FRT recombination sites in place of the wild type E. coli BW25113's pyrF gene, which was deleted by genetic engineering to create a strain without the spectinomycin resistance marker.

### Integration of the T7 Polymerase Cassette

In order to create a strain with inducible expression of M13 phage genes, the T7 polymerase gene under control of an inducible promoter (the IPTG-inducible lac promoter) was inserted at the endA locus using recombineering and CRISPR negative selection. The endA locus was chosen as the insertion site because EndA protein is known to degrade DNA and deleting endA is known to increase yield and quality of DNA produced in E. coli. Lin, J J (1992) Endonuclease A degrades chromosomal and plasmid DNA of Escherichia coli present in most preparations of single stranded DNA from phagemids. Proc. Natl. Sci. Counc. Repub. China B 16 1-5. Correct integrants were identified through colony PCR and Oxford Nanopore sequencing.

Having integrated an inducible T7 polymerase expression cassette, we next verified that the new strain, bac035 could in-fact induce expression of T7 polymerase and that this polymerase could transcribe genes from the T7 promoter. To test this, the strain was integrated with a pT7:emGFP expression cassette (containing a nucleotide sequence for emGFP expression driven by pT7 promoter). Specifically, strain bac036 was created by integrating the pLac:T7 polymerase from E. coli BL21 (DE3) into the endA locus of the BW25113 ΔpyrF background strain. A cassette expressing emGFP from the T7 promoter was then inserted yhaV to create strain bac058. This strain was grown in various concentrations of IPTG to induce expression of the T7 polymerase and by proxy, emGFP. Cultures were grown at 30° C. for 48 hours in a shaking incubator. Absorbance at 600 nm (OD600) and GFP fluorescence were measured at the conclusion of the growth period in a Biotek Synergy H1 plate reader. The resulting arbitrary fluorescence units were divided by OD600 and compared to the parent strain bac036 which lacked the GFP expression plasmid (FIG. 13B). As shown in FIG. 13B, the results demonstrated induction of pLac:T7 polymerase expression.

### Integration of the First Transcriptional Unit

The first transcriptional unit of the M13 genome (TU1) was integrated into strain bac036 using recombineering and antibiotic selection. TU1 was cloned into four circular, double-stranded plasmids cdsDNA206, cdsDNA207, cdsDNA208 and cdsDNA209 along with homology arms for integration into the E. coli BW25113 genome at the intA locus, and a spectinomycin selectable marker to allow for selection of integrants. TU1 comprises M13 genes II, V, VII, VIII, and IX. Since the optimal expression level of the integrated M13 genes was not known a priori, each of the four plasmids had a promoter of a different strength that expressed mRNA at a different level. cdsDNA206 had a medium strength T7 promoter, cdsDNA207 had a weak T7 promoter, cdsDNA208 had the wild type M13 TU1 promoter and cdsDNA209 had a strong T7 promoter. The T7 promoters were mutated from the wild type T7 promoter to achieve these different expression levels based on data in Komura et al., High-throughput evaluation of T7 promoter variants using biased randomization and DNA barcoding, Plos, 2018. The plasmid contains a p15A origin for replication in E. coli and an ampicillin resistance marker for selection of the plasmid in E. coli. The spectinomycin selectable marker was flanked by FRT recombination sites to allow marker recycling upon expression of the Flp recombinase.

Bac090-bac092 expressed the T7 polymerase from the lac promoter and M13 TU1 from three different promoters (integrated into the E. coli BW25113 genome at the intA locus); a strong T7 promoter, a weak T7 promoter and the native M13 TU1 promoter respectively.

### Integration of the Second Transcriptional Unit

The second transcriptional unit (TU2) was then integrated into strains bac090-092 at the intZ locus to create a small, combinatorial library of different combinations of TU1 and TU2 expression levels. TU2 comprises M13 genes III, VI, I, and IV.

Four different promoters were tested to drive expression of TU2. In cdsDNA215 TU2 is driven by a strong T7 promoter; in cdsDNA216 TU2 is driven by a medium T7 promoter; in cdsDNA217 TU2 is driven by a weak T7 promoter and in cdsDNA218 TU2 is driven by its wild type M13 TU2 promoter. The intZ gene was chosen as the integration locus because it is a prophage associated integrase that has no known beneficial function to E. coli. The resulting new strains were labeled bac105-110 and glycerol stocks were stored at −80 C.

Having integrated the IPTG inducible T7 polymerase cassette and a small library of promoters driving both M13 TU1 and M13 TU2 into the same strain, we now had a set of strains which contained all the genes of the M13 genome and a T7 polymerase and promoter system.

Assaying the New Strains for cssDNA Production

Having created a small library of strains expressing M13 TU1 and M13 TU2 from promoters of different strengths under control of an IPTG inducible T7 polymerase, we next sought to assay the new strains for their ability to produce cssDNA.

First, strains bac105-bac110 were transformed by electroporation with phagemid cdsDNA111, which contains a F1 origin of replication, F1 packaging signal, and an ampicillin resistance gene and RFP expression cassette. Single colonies were picked in triplicate and inoculated into 500 ul LB supplemented with 50 ug/ml kanamycin and grown overnight at 37 C. In the morning, the optical density at 600 nm (OD600) was measured and the cultures were diluted to OD 0.05 in 200 μl Riesenberg defined medium. This culture was then incubated until saturation at 30 C which took approximately 48 hours at 30 C.

The culture was centrifuged at 4000 RCF to remove E. coli cells containing double-stranded plasmid DNA and the supernatant which contained the phage particles was collected for analysis. The phage particles were then lysed by heating the solution to 99 C for 10 min. The supernatant containing the cssDNA was retained for use as a qPCR template. cssDNA was quantitated using qPCR calibrated using a standard curve of cdsDNA111 DNA.

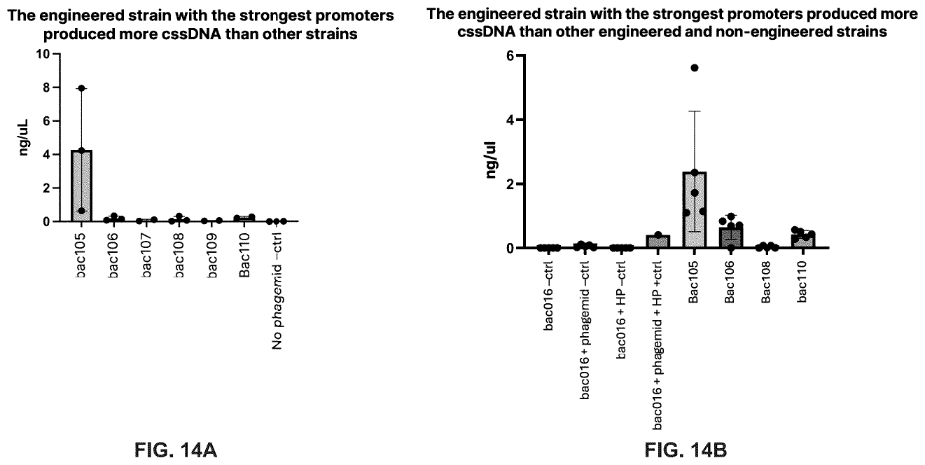

FIG. 14A shows the results of a screen of a small combinatorial library of integrated TU1 and TU2 driven by different strength promoters. Strains with integrated T7 polymerase, M13 TU1 and M13 TU2 were transformed with phagemid cdsDNA111 to create hosts theoretically capable of producing cssDNA. Single colonies were picked in quintuplicate and inoculated into 500 μl LB Miller supplemented with carbenicillin to select for the phagemid and grown overnight as precultures. These cultures were then diluted to OD600 0.05 and incubated on a plate for approximately 48 hours until saturation. The supernatant was harvested and phage particles were lysed releasing their cssDNA for use as qPCR templates. cssDNA was quantitated using qPCR primers targeting the F1 ori present on the cssDNA and a standard curve cdsDNA111 DNA. Bac105 expressing both M13 TU1 and TU2 from strong T7 promoters produced the most cssDNA. Other strains with weaker promoter combinations did not produce significantly more DNA than the negative control which lacked a phagemid that could be used as a template for cssDNA production.

Next, we desired to replicate the above data in comparison with other production hosts that lack integrated M13 genes. As a control, the parental strain bac016 (E. coli BW25113 ΔpyrF) and the standard DH5α cssDNA production host, bac001 were transformed with the same cdsDNA111 phagemid and a helper plasmid (cdsDNA114) which is derived from helper phage M13KO7 by removing the F1 origin and the packaging signal by amplifying the kanamycin marker and all of the protein coding genes from M13KO7 with primers designed to exclude the F1 origin of replication and the packaging signal and then re-circularizing the amplified DNA using a DNA ligase in vitro reaction.

FIG. 14B shows the results comparing select engineered strains to their non-engineered parent strain. Engineered production hosts expressing M13 phage genes from their genomes were transformed with cdsDNA111 phagemid. The non-engineered parent strain bac016 was transformed with the helper plasmid cdsDNA114 (“HP” in FIG. 14B) to provide expression of M13 phage genes and the same cdsDNA111 phagemid. Transformed colonies were inoculated in quintuplicate and grown for 48 h in 200 uL of medium in a 96-well microplate at 30 C with appropriate antibiotics for plasmid maintenance. The supernatant was then collected by centrifugation to remove E. coli and boiled at 99 C for 10 min to lyse phage particles. It was then used as a template for qPCR to quantitate cssDNA production. Standard curves were used for qPCR measuring the presence of the F1 Ori in solution as a proxy for cssDNA concentration. As in the previous experiment, bac105 expressing M13 TU1 and TU2 from the strongest pair of promoters outperformed the other three engineered strains tested (bac106, bac108 and bac110) which all had weaker promoters driving expression of the M13 genes. Notably, bac105 outperformed its parent strain bac016 which utilized the traditional two-plasmid production system rather than integrated phage genes like bac105. Very little to no cssDNA was detected in any of the three negative control strains which lacked complete cssDNA production systems.

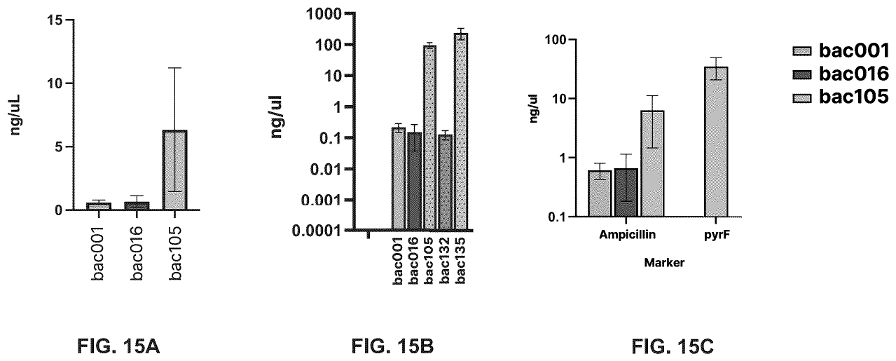

FIG. 15A shows a comparison of best engineered production host (bac105) to its parent strain and to the traditional production strain DH5α using the traditional helper plasmid system. All three strains were transformed with the phagemid cdsDNA111 to serve as a cssDNA production template. The non-engineered DH5α and BW25113 strains bac001 and bac016 were also transformed with the helper plasmid cdsDNA114 to provide expression of the M13 phage genes. Strains were grown for 48 h in 200 uL of medium in a microplate at 30 C with appropriate antibiotics for plasmid maintenance. The supernatant was collected by centrifugation to remove E. coli and boiled at 99 C for 10 min to lyse phage particles. It was then used as a template for qPCR to quantitate cssDNA production. Standard curves were used for qPCR measuring the presence of the F1 Ori in solution as a proxy for cssDNA concentration. The engineered bac105 strain expressing M13 integrated copies of TU1 and TU2 from strong T7 promoters produced more cssDNA than either of the two strains expressing the M13 phage genes from the helper plasmid. Notably bac016 was the parent strain to bac105 so differences in cssDNA production are likely due to differences in M13 gene expression rather than differences in strain background. Also notably, bac001 with similar helper plasmids and phagemids is a common cssDNA production strain in industrial and academic settings.

### Integration of pT7 Driving Individual M13 Gene VIII

It was hypothesized that the native stoichiometry of the various M13 genes present in the wild type M13 genome may not be optimal for high levels of cssDNA production especially for cssDNA constructs that differ significantly from the M13 genome in qualities such as length and GC content. For example, the M13 shaft is made up of thousands of copies of the gene VIII protein product. The shaft lengthens to accommodate longer cssDNA constructs and contracts to accommodate shorter ones. Production of cssDNA constructs that are significantly different in length of the 7 KB M13 wild type genome may be improved by different ratios of gene VIII expression relative to other M13 genes.

Strains bac132 (bac105 plus gene II integrated into the mazF locus) and bac135 (bac105 plus gene VIII integrated into the mazF locus) were generated. The mazF locus was chosen as an integration locus because it is the toxin portion of the mazE/mazF toxin/anti-toxin system. It was thought that deleting the toxic gene, mazF may have beneficial effects to the strain.

FIG. 15B shows the results of an assay of cssDNA production across engineered strains with additional individual phage genes integrated. Bac001 (non-engineered DH5α), bac016 (Non-engineered BW25113), bac105 (best engineered strain so far), bac132 (bac105 plus gene II) and bac135 (bac105 plus gene VIII) were each transformed with the cdsDNA111 RFP phagemid and grown for 48 hours in Riesenberg defined medium supplemented with uracil. The broth was harvested and centrifuged to clear the E. coli cells from the culture. The supernatant was transferred to clean tube and heated at 99 C for 10 minutes to lyse the phage particles and release the cssDNA into the medium. This solution was used as a template for qPCR with primers targeting the RFP gene and a standard curve of cdsDNA111 RFP phagemid. The engineered strain bac105 again outperformed the non-engineered control strains. Its daughter strain bac132 an extra copy of gene II integrated performed poorly but the other daughter strain bac135 with an extra copy of gene VIII inserted performed slightly better than its parent bac105. Since the difference was small, we chose to compare the two strains bac105 and bac135 against each other in future experiments.

Having identified strain bac135 which includes the T7 polymerase, TU1, TU2 and an individual copy of gene VIII as the best producer out of the two strains that had an individual M13 gene integrated, we next sought to test production of alternative cssDNA sequences besides the RFP sequence from cdsDNA111. One hypothesis had been that adding extra copies of gene VIII would allow the cells to produce more cssDNA especially when the cssDNA sequence was significantly longer than the 7 kb genome of the wild type M13 genome. This is because the shaft of the M13 phage capsid which is made up of thousands of copies of p8 (the product of gene VIII) expands and contracts to accommodate different lengths of cssDNA sequences. Therefore, longer cssDNA sequences would require more copies of p8 protein in order to be packaged into the capsid making it logical that more gene VIII expression would facilitate production of longer cssDNA sequences.

### Example 9: Production of cssDNA Encoding Utrophin and Dystrophin

This example demonstrates the ability of the engineered production strains described in Example 8 to produce cssDNA containing the long genetic sequences coding for dystrophin and utrophin. The engineered cssDNA system advantageously allows for production of long cssDNA templates which are useful for gene therapies.

Strains were assayed by transforming with phagemids cdsDNA100 (utrophin), cdsDNA101 (dystrophin). Non-engineered control strains bac001 and bac016 were also transformed with the helper plasmid cdsDNA114 to provide expression of the M13 phage genes. Transformed colonies were inoculated in triplicate and grown for 48 h in 200 uL of media in a 96-well microplate at 30 C with appropriate antibiotics for plasmid maintenance. The broth was then harvested by centrifugation to remove E. coli cells and the supernatant containing the phage particles was collected and boiled at 99 C for 10 min to lyse the phage particles. It was then used as a template for qPCR to quantitate cssDNA production. Standard curves were used for qPCR measuring the presence the AAVS1 homology arm sequence in solution as a proxy for cssDNA concentration. FIG. 15B shows the results for production of various cssDNA sequences from engineered host strains.

Bac001 (traditional DH5α), bac016 (non-engineered parent), bac105 (best engineered strain to date) and bac135 (bac105 plus and extra copy of gene VIII) were assayed. The two engineered strains bac105 and bac135 produced more cssDNA than their non-engineered comparators and did so more consistently with a smaller standard deviation between bioreps. Notably, both the utrophin and dystrophin constructs are therapeutically relevant for the treatment of Duchenne Muscular Dystrophy and each construct is longer than 13 kb when produced as cssDNA. Utrophin can be produced at low levels using the traditional helper plasmid system but dystrophin cannot whereas each can be produced at moderate levels using the engineered strains. The integration of an additional copy of gene VIII seemed to increase utrophin cssDNA yield slightly.

### Example 10: Production of cssDNA Using a Phagemid with an Auxotrophic Marker

The standard phagemid, cdsDNA111 was amplified with PCR primers designed to amplify the entire the plasmid excluding the ampicillin resistance cassette. The pyrF gene cassette was amplified from the E. coli DH5α genome along with 75 bp of its native promoter. In both PCR reactions, the primers were designed with tails that produced 30 nt overlaps with the other fragment. These two linear double-stranded DNA fragments were assembled using a NEBuilder HiFi DNA assembly enzyme mix (catalogue #E2621L, NEB, Ipswich, MA). This new plasmid was sequence confirmed using Oxford Nanopore technology and stored as cdsDNA117.

The F1 origin of replication is included in the design so that the phagemid can replicate as cssDNA in E. coli. The packaging signal is included so that the M13 proteins will package the cssDNA into the phage particle. The pUC19 origin of replication is included so that the phagemid can replicate as dsDNA in E. coli. The pyrF gene is used as a selectable auxotrophic marker for use in the engineered production and its descendants which are uracil auxotrophs due to the disruption of the genomic copy of the pyrF gene. Golden Gate Assembly™ sites (Engler et al., PLoS One. 2008; 3(11):e3647) were included in this phagemid. The Golden Gate Assembly™ sites can be recognized by the BsmBI Type II restriction enzyme allowing easy insertion of new user-defined sequences into the pyrF phagemid backbone. User defined sequences can be synthesized or amplified with primers such that they are flanked by BsmBI restriction sites. The insert and the phagemid are then incubated together with the BsmBI restriction enzyme and buffer and ligase and the temperature is cycled between 37° C. for restriction enzyme cutting and 16 C for ligation. Because the restriction enzyme recognition sequences are asymmetrical and are a few bases away from the cut site, the recognition sequences are inserted into the phagemid and the inserts in such a way that they are cleaved off after the initial digestion. If the parts then assemble in the desired manner, inserting the user defined sequence into the phagemid, then the recognition sequence is ablated and no more cutting can ensue. If, however, the fragments re-ligate to the recognition sequence reforming the initial inputs, the recognition sequence is recreated and another round of cutting will ensue. In this way the reaction proceeds unidirectionally until almost all restriction recognition sites have been abolished and almost all of the inserts have been correctly inserted into the phagemid backbone.

Notably, the pyrF auxotrophic marker is included in the phagemid in place of traditional antibiotic markers to increase the safety of the cssDNA produced from the system. Since one of the applications of the cssDNA produced from this system is human therapeutic gene and cell therapy, it is not desirable to include antibiotic marker sequences. The exclusion of antibiotic marker sequences prevents this system from spreading antibiotic resistance to microbes that may infect human patients with antibiotic resistant strains.

The engineered production host, bac105 was transformed with phagemids cdsDNA111 (RFP with ampicillin resistance marker) and cdsDNA117 (RFP with a pyrF auxotrophic marker). Non-engineered control strains bac001 (DH5α) and bac016 (BW25113) were transformed with phagemid cdsDNA111 and with helper plasmid cdsDNA114 to provide expression of the M13 phage genes. Strains were grown for 48 hours in 200 uL of defined medium in a microplate at 30 C with appropriate antibiotics or nucleoside dropouts for plasmid maintenance. The engineered strain harboring the phagemid with the auxotrophic marker was grown in defined medium without uracil. Others were grown in defined medium with uracil. The broth was harvested and the supernatant including the phage particles was clarified by centrifugation to remove E. coli. The supernatant was heated at 99 C for 10 min to lyse the phage particles containing the cssDNA and this was used as a template for qPCR to quantitate cssDNA production. Standard curves of cdsDNA111 containing the RFP sequence were used. qPCR primers targeted the RFP gene insert of the cssDNA. Parental strain bac016 with only helper plasmid cdsDNA114 and with only phagemid cdsDNA111 were included as negative controls.

The results are shown in FIG. 15C. When transformed with the RFP/ampicillin phagemid cdsDNA111, bac105 once again outperformed its parent strain bac016 and the traditional production host bac001 when transformed with the same phagemid (FIG. 15C). Notably, bac105 also outperformed those two strains when it was transformed with the RFP/pyrF phagemid cdsDNA117 (FIG. 15C). A direct comparison of the engineered strain bac105 to top the non-engineered traditional production host bac001 (DH5α) was not performed because this strain lacks a uracil auxotrophy and offers no way to select for the pyrF marker.

### CONCLUSION

This high cssDNA production from the engineered strain using a phagemid template with an auxotrophic marker establishes bac105 as the safest cssDNA production host available. It produces more cssDNA than traditional helper plasmid or helper phage systems without creating infectious phage particles because the M13 origin of replication and packaging signal reside on the phagemid which lacks the genes necessary to replicate itself. Furthermore, the chances of an infectious phage evolving out of this production system are greatly reduced by the fact that the M13 genes necessary for phage replication are stably integrated in the E. coli genome at two separate loci, meaning there would need to be two separate recombination events in order for the two sets of M13 genes to recombine with each other and with the phagemid to reunite all M13 genes with the M13 origin of replication and packaging signal. Furthermore, the ability of this strain to produce cssDNA from phagemid templates with auxotrophic markers means that the entire strain is free of any antibiotic markers during production and no antibiotics need to be added to the production medium. The reduces the possibility of evolving antibiotic resistant bacteria either during the cssDNA production process or later when the cssDNA is administered to patients. Lastly, the engineered production host grows well in defined medium obviating the need for expensive rich media which produce less consistent run to run results.

TABLE 5
Engineered strains produced in this study

| Re- | | |
|---|---|---|
| gistry | Back- | |
| ID | ground | Genotype |
| bac016 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF |
| bac026 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + cdsDNA167 |
| | | Cas9 recombineering plasmid |
| bac035 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + cdsDNA167 |
| | | Cas9 recombineering plasmid + T7 |
| | | polymerase at endA |
| bac036 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | at endA with plasmids evicted |
| bac058 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmB-3) ΔKan::pyrF + |
| | | Cas9 recombineering plasmid + T7 polymerase |
| | | integration + pT7:GFP integration |
| bac060 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 polymerase |
| | | integration + pT7 low:TU1 |
| bac061 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 polymerase |
| | | integration + pWT:TU1 |
| bac062 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 polymerase |
| | | integration + pT7 low:TU1 |
| bac063 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 polymerase |
| | | integration + pT7 high:TU1 |
| bac064 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 polymerase |
| | | integration + pT7 low:TU1 |
| bac065 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pWT:TU1 |
| bac072 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low:TU1 ΔSmR |
| bac073 | BW25113 | lacI+rrBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rrnB-3) ΔKan::pyrF + Cas9 |
| | | recombineering plasmid + T7 polymerase |
| | | integration + pT7 low:TU1 ΔSmR |
| bac074 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low:TU1 ΔSmR |
| bac075 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low: TU1 ΔSmR |
| bac076 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low: TU1 ΔSmR |
| bac077 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low:TU1 ΔSmR |
| bac078 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 low:TU1 ΔSmR |
| bac079 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rrnB-3) ΔKan::pyrF + |
| | | Cas9 recombineering plasmid + T7 |
| | | polymerase integration + pT7 low:TU1 ΔSmR |
| bac080 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 low:TU1 ΔSmR |
| bac081 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmB-3) ΔKan::pyrF + Cas9 |
| | | recombineering plasmid + T7 polymerase |
| | | integration + pT7 low:TU1 ΔSmR |
| bac082 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 high: TU1 ΔSmR |
| bac083 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + Cas9 |
| | | recombineering plasmid + T7 polymerase |
| | | integration + pT7 high: TU1 ΔSmR |
| bac084 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + |
| | | pT7 low: TU1 ΔSmR cured of the Flp recombinase |
| bac085 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low: |
| | | TU1 ΔSmR cured of the Flp recombinase |
| bac086 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low: |
| | | TUI ΔSmR cured of the Flp recombinase |
| bac087 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pWT: |
| | | TU1 ΔSmR cured of the Flp recombinase |
| bac088 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rrnB-3) ΔKan::pyrF + |
| | | Cas9 recombineering |
| | | plasmid + T7 polymerase integration + pWT: |
| | | TU1 ΔSmR cured of the Flp recombinase |
| bac089 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 high: |
| | | TU1 ΔSmR cured of the Flp recombinase |
| bac090 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 high: |
| | | TU1 ΔSmR + cdsDNA167 Cas9 |
| | | recombineering plasmid |
| bac091 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low:TU1 |
| | | ΔSmR + cdsDNA167 Cas9 recombineering plasmid |
| bac092 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pWT:TU1 |
| | | ΔSmR + cdsDNA167 Cas9 recombineering plasmid |
| bac 105 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 high: |
| | | TU1 pT7 high:TU2 cured of all plasmids |
| bac 106 | BW25113 | lacI+rrBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low: |
| | | TU1 pT7 high:TU2 cured of all plasmids |
| bac 107 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low:TU1 |
| | | pT7 medium:TU2 cured of all plasmids |
| bac 108 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pT7 low:TU1 |
| | | pT7 low:TU2 cured of all plasmids |
| bac 109 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pWT:TU1 |
| | | pT7 high:TU2 cured of all plasmids |
| bac110 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering |
| | | plasmid + T7 polymerase integration + pWT:TU1 |
| | | pT7 medium:TU2 cured of all plasmids |
| bac 132 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene II cured of all plasmids |
| bac133 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene II cured of all plasmids |
| bac134 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene II cured of all plasmids |
| bac135 | BW25113 | lacI+rmnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene vIII |
| | | cured of all plasmids |
| bac136 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene vIII |
| | | cured of all plasmids |
| bac137 | BW25113 | lacI+rrnBT14 ΔlacZWJ16 hsdR514 ΔaraBADAH33 |
| | | ΔrhaBADLD78 rph-1 Δ(araB−D)567 Δ(rhaD−B)568 |
| | | ΔlacZ4787(::rmnB-3) ΔKan::pyrF + |
| | | Cas9 recombineering plasmid + T7 |
| | | polymerase integration + pT7 high:TU1 |
| | | pT7 high:TU2 pT7 high:gene vIII cured |
| | | of all plasmids |

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

EXEMPLARY SEQUENCES

| SEQ | | |
|---|---|---|
| ID NO | Description | Sequence |
| 1 | wild type T7 | Taatacgactcactatagggcgaattgggtaccgggcccagaccacaacggtttccctctag |
| | | aaataattttgtttaactttaagaaggagatatacat |
| 2 | Sequence 7834, | taatccgactcactatagggcgaattgggtaccgggcccagaccacaacggtttccctctag |
| | Komura et al., High- | aaataattttgtttaactttaagaaggagatatacat |
| | throughput | |
| | evaluation of T7 | |
| | promoter variants | |
| | using biased | |
| | randomization and | |
| | DNA barcoding, | |
| | Plos, 2018 | |
| 3 | Sequence 7812, | Gaatacgactcactatagcgcgaattgggtaccgggcccagaccacaacggtttccctctag |
| | Komura et al., High- | aaataattttgtttaactttaagaaggagatatacat |
| | throughput | |
| | evaluation of T7 | |
| | promoter variants | |
| | using biased | |
| | randomization and | |
| | DNA barcoding, | |
| | Plos, 2018 | |
| 4 | p1 phage protein | MAVYFVTGKLGSGKTLVSVGKIQDKIVAGCKIATNLDLRLQNLPQVGR |
| | | FAKTPRVLRIPDKPSISDLLAIGRGNDSYDENKNGLLVLDECGTWFNTR |
| | | SWNDKERQPIIDWFLHARKLGWDIIFLVQDLSIVDKQARSALAEHVVY |
| | | CRRLDRITLPFVGTLYSLITGSKMPLPKLHVGVVKYGDSQLSPTVERWL |
| | | YTGKNLYNAYDTKQAFSSNYDSGVYSYLTPYLSHGRYFKPLNLGQKM |
| | | KLTKIYLKKFSRVLCLAIGFASAFTYSYITQPKPEVKKVVSQTYDFDKFT |
| | | IDSSQRLNLSYRYVFKDSKGKLINSDDLQKQGYSLTYIDLCTVSIKKGNS |
| | | NEIVKCN |
| 5 | p2 phage protein | MIDMLVLRLPFIDSLVCSRLSGNDLIAFVDLSKIATLSGMNLSARTVEYH |
| | | IDGDLTVSGLSHPFESLPTHYSGIAFKIYEGSKNFYPCVEIKASPAKVLQ |
| | | GHNVFGTTDLALCSEALLLNFANSLPCLYDLLDVNATTISRIDATFSAR |
| | | APNENIAKQVIDHLRNVSNGQTKSTRSQNWESTVTWNETSRHRTLVAY |
| | | LKHVELQHQIQQLSSKPSAKMTSYQKEQLKVLSNPDLLEFASGLVRFEA |
| | | RIKTRYLKSFGLPLNLFDAIRFASDYNSQGKDLIFDLWSFSFSELFKAFE |
| | | GDSMNIYDDSAVLDAIQSKHFTITPSGKTSFAKASRYFGFYRRLVNEGY |
| | | DSVALTMPRNSFWRYVSALVECGIPKSQLMNLSTCNNVVPLVRFINVD |
| | | FSSQRPDWYNEPVLKIA |
| 6 | p3 phage protein | MKKLLFAIPLVVPFYSHSAETVESCLAKPHTENSFTNVWKDDKTLDRY |
| | | ANYEGCLWNATGVVVCTGDETQCYGTWVPIGLAIPENEGGGSEGGGS |
| | | EGGGSEGGGTKPPEYGDTPIPGYTYINPLDGTYPPGTEQNPANPNPSLEE |
| | | SQPLNTFMFQNNRFRNRQGALTVYTGTVTQGTDPVKTYYQYTPVSSK |
| | | AMYDAYWNGKFRDCAFHSGFNEDPFVCEYQGQSSDLPQPPVNAGGGS |
| | | GGGSGGGSEGGGSEGGGSEGGGSEGGGSGGGSGSGDFDYEKMANAN |
| | | KGAMTENADENALQSDAKGKLDSVATDYGAAIDGFIGDVSGLANGNG |
| | | ATGDFAGS |
| 7 | p4 phage protein | MKLLNVINFVFLMFVSSSSFAQVIEMNNSPLRDFVTWYSKQSGESVIVS |
| | | PDVKGTVTVYSSDVKPENLRNFFISVLRANNFDMVGSIPSIIQKYNPNN |
| | | QDYIDELPSSDNQEYDDNSAPSGGFFVPQNDNVTQTFKINNVRAKDLIR |
| | | VVELFVKSNTSKSSNVLSIDGSNLLVVSAPKDILDNLPQFLSTVDLPTDQ |
| | | ILIEGLIFEVQQGDALDFSFAAGSQRGTVAGGVNTDRLTSVLSSAGGSF |
| | | GIFNGDVLGLSVRALKTNSHSKILSVPRILTLSGQKGSISVGQNVPFITGR |
| | | VTGESANVNNPFQTIERQNVGISMSVFPVAMAGGNIVLDITSKADSLSS |
| | | STQASDVITNQRSIATTVNLRDGQTLLLGGLTDYKNTSQDSGVPFLSKIP |
| | | LIGLLFSSRSDSNEESTLYVLVKATIVRAL |
| 8 | p5 phage protein | MIKVEIKPSQAQFTTRSGVSRQGKPYSLNEQLCYVDLGNEYPVLV |
| | | KITLDEGQPAYAPGLYTVHLSSFKVGQFGSLMIDRLRLVPAK |
| 9 | p6 phage protein | MPVLLGIPLLLRFLGFLLVTLFGYLLTFLKKGFGKIAIAISLFLALIIGLNSI |
| | | LVGYLSDISAQLPSDFVQGVQLILPSNALPCFYVILSVKAAIFIFDVKQKI |
| | | VSYLDWDK |
| 10 | p7 phage protein | MEQVADFDTIYQAMIQISVVLCFALGIIAGGQR |
| 11 | p8 phage protein | MKKSLVLKASVAVATLVPMLSFAAEGDDPAKAAFNSLQASATEYIGY |
| | | AWAMVVVIVGATIGIKLFKKFTSKAS |
| 12 | p9 phage protein | MSVLVYSFASFVLGWCLRSGITYFTRLMETSS |
| 13 | p10 phage protein | MNIYDDSAVLDAIQSKHFTITPSGKTSFAKASRYFGFYRRLVNEGYDSV |
| | | ALTMPRNSFWRYVSALVECGIPKSQLMNLSTCNNVVPLVRFINVDESSQ |
| | | RPDWYNEPVLKIA |
| 14 | p11 phage protein | MKLTKIYLKKFSRVLCLAIGFASAFTYSYITQPKPEVKKVVSQTYDFDK |
| | | FTIDSSQRLNLSYRYVFKDSKGKLINSDDLQKQGYSLTYIDLCTVSIKKG |
| | | NSNEIVKCN |
| 15 | NP_510893.1 | taatacgactcactatagggcgaattgggtaccgggcccagaccacaacggtttccctctag |
| | M13 genes I and XI | aaataattttgtttaactttaagaaggagatatacatATGGCTGTTTATTTTGTAACAGGAA |
| | (upon integration | AACTAGGCTCCGGCAAGACCTTAGTCAGCGTTGGTAAGATCCAGGATAAGATCGT |
| | into KTx00004 | GGCCGGTTGTAAAATTGCCACCAACCTGGATCTTCGCCTGCAAAACC |
| | termed | TGCCACAGGTTGGCCGTTTTGCGAAAACCCCGCGTGTTCTGCGTATT |
| | KTx00004.1.11) | CCGGATAAGCCGAGCATCAGCGACTTGCTGGCGATCGGTCGCGGCA |
| | | ACGACTCGTACGACGAGAATAAAAACGGTCTGCTGGTGTTGGACGA |
| | | GTGCGGTACATGGTTCAACACCCGTTCTTGGAATGATAAAGAAAGA |
| | | CAACCGATCATTGACTGGTTCCTGCACGCTCGCAAATTGGGTTGGGA |
| | | TATCATCTTTCTGGTCCAGGACCTGTCAATCGTGGACAAACAAGCAC |
| | | GTTCCGCGCTGGCTGAACATGTGGTGTACTGCCGTCGTCTGGACCGT |
| | | ATCACCCTGCCGTTCGTGGGCACCCTGTACTCTCTGATTACGGGTAG |
| | | CAAGATGCCGCTGCCGAAACTGCACGTCGGTGTGGTTAAGTATGGT |
| | | GATAGCCAGCTTTCCCCGACCGTCGAGCGCTGGCTGTATACCGGTAA |
| | | GAATCTTTACAATGCATATGATACGAAGCAAGCGTTTAGCTCTAACT |
| | | ATGACTCCGGTGTTTATTCGTACCTCACTCCGTATTTGAGCCATGGT |
| | | CGTTATTTCAAACCGCTGAACTTGGGCCAGAAAATGAAATTGACCA |
| | | AAATTTATCTGAAAAAATTCAGCCGTGTTCTTTGCTTAGCGATCGGC |
| | | TTCGCAAGCGCGTTTACGTACAGCTACATTACGCAGCCGAAACCCG |
| | | AAGTTAAAAAAGTTGTAAGCCAGACCTACGACTTTGATAAGTTCAC |
| | | CATTGACTCCTCCCAACGTCTGAATCTGTCGTACCGCTACGTGTTTA |
| | | AGGATAGTAAGGGCAAGCTCATCAACAGCGATGATTTACAGAAGCA |
| | | AGGTTATAGCTTGACCTACATTGACTTGTGTACCGTTTCTATCAAAA |
| | | AGGGAAACAGCAACGAGATTGTGAAGTGCAATTAAggatccggctgctaaca |
| | | aagcccgaaaggaagctgagttggctgctgccaccgctgagcaataactagcataacccctt |
| | | ggggcctctaaacgggtcttgaggggttttttgACAAAGAGGGCGAGCGTTAA |
| 16 | NP_510885.1 | taatacgactcactatagggcgaattgggtaccgggcccagaccacaacggtttccctctag |
| | M13 genes II and 10 | aaataattttgtttaactttaagaaggagatatacatATGATAGATATGTTAGTATTGAGGC |
| | KTx00004.2.10 | TACCCTTCATCGACAGTCTTGTGTGCTCACGTTTGTCGGGAAACGATCTGATCGCG |
| | | TTCGTGGACTTGTCTAAAATTGCTACTCTGTCTGGTATGAACCTGAG |
| | | CGCGCGTACTGTAGAGTACCATATTGATGGTGACTTGACCGTTAGCG |
| | | GCCTGTCCCACCCGTTTGAAAGCTTGCCGACCCATTATAGCGGCATC |
| | | GCGTTCAAAATCTACGAAGGTTCCAAGAACTTTTACCCGTGTGTTGA |
| | | GATTAAGGCGTCCCCCGCAAAAGTTCTGCAAGGTCACAACGTCTTTG |
| | | GTACGACCGATCTCGCCCTCTGTAGCGAAGCACTGCTGCTAAACTTT |
| | | GCTAATAGTTTACCGTGCCTGTATGACTTGCTGGACGTGAATGCAAC |
| | | CACCATCAGCCGCATTGATGCGACGTTTAGCGCGCGCGCACCGAAT |
| | | GAAAATATCGCGAAACAGGTTATCGACCACCTGCGTAACGTTAGCA |
| | | ACGGTCAGACCAAAAGCACCCGTTCTCAAAACTGGGAAAGCACTGT |
| | | CACCTGGAATGAAACCTCCCGTCATCGTACCCTGGTCGCATATTTGA |
| | | AGCACGTGGAGCTGCAACATCAGATTCAACAGTTGTCCTCTAAGCC |
| | | GAGCGCTAAGATGACCAGCTATCAGAAGGAACAGTTGAAGGTGTTG |
| | | AGCAATCCGGACCTGTTGGAATTTGCGAGTGGCCTGGTGCGTTTCGA |
| | | GGCGCGCATCAAAACCAGATACCTGAAGAGCTTTGGCCTGCCACTG |
| | | AACCTGTTCGATGCGATTCGTTTTGCGTCTGATTACAACTCGCAAGG |
| | | TAAAGATTTAATCTTTGATTTATGGAGCTTCTCCTTCAGCGAGCTATT |
| | | CAAAGCCTTCGAGGGTGACTCTATGAATATCTATGATGATAGCGCG |
| | | GTCCTGGACGCCATCCAAAGCAAACACTTTACGATTACCCCGAGCG |
| | | GCAAGACCTCGTTCGCCAAGGCTAGCCGCTACTTCGGTTTTTACCGC |
| | | CGTCTGGTTAACGAGGGCTACGACTCAGTTGCACTGACCATGCCGC |
| | | GTAACTCCTTCTGGCGTTACGTGTCCGCTCTGGTGGAGTGCGGCATT |
| | | CCGAAAAGCCAGCTGATGAACCTGTCTACGTGCAACAACGTTGTTC |
| | | CACTCGTGCGCTTCATTAATGTGGACTTTTCCTCTCAGCGTCCGGAC |
| | | TGGTATAACGAGCCGGTTCTGAAAATCGCGTAAggatccggctgctaacaaagc |
| | | ccgaaaggaagctgagttggctgctgccaccgctgagcaataactagcataaccccttgggg |
| | | cctctaaacgggtcttgaggggttttttgACAAAGAGGGCGAGCGTTAA |
| 17 | NP_510891.1 | taatacgactcactatagggcgaattgggtaccgggcccagaccacaacggtttccctctag |
| | M13 gene III | aaataattttgtttaactttaagaaggagatatacatATGAAAAAGTTATTGTTTGCTATAC |
| | KTx00004.3 | CCCTAGTCGTTCCGTTTTATAGCCACAGCGCGGAAACCGTGGAATCTTGTCTGGCG |
| | | AAACCGCATACGGAAAACTCTTTTACCAATGTGTGGAAAGATGATA |
| | | AGACCCTGGATAGATATGCGAACTATGAGGGTTGTCTTTGGAATGC |
| | | GACCGGCGTGGTGGTGTGCACCGGCGACGAAACCCAATGTTATGGT |
| | | ACTTGGGTTCCGATTGGTTTGGCGATCCCGGAGAACGAGGGCGGGG |
| | | GCTCTGAGGGCGGCGGTAGCGAGGGTGGCGGTTCCGAGGGTGGAGG |
| | | TACCAAACCGCCGGAATACGGTGACACCCCGATCCCGGGTTATACG |
| | | TACATCAACCCGTTGGACGGCACGTACCCGCCAGGTACTGAGCAGA |
| | | ATCCGGCAAACCCAAATCCGAGCCTGGAAGAGAGCCAACCGTTGAA |
| | | TACCTTCATGTTTCAGAACAACCGCTTCCGCAACCGTCAGGGTGCGC |
| | | TGACCGTTTATACCGGTACAGTTACCCAGGGTACGGATCCGGTGAA |
| | | AACCTACTATCAGTACACTCCGGTCAGCTCGAAAGCCATGTACGAT |
| | | GCGTATTGGAACGGTAAGTTCCGCGATTGCGCCTTCCACAGCGGCTT |
| | | CAACGAAGATCCGTTCGTGTGCGAATACCAAGGTCAGTCGAGCGAC |
| | | CTGCCGCAGCCGCCCGTTAATGCTGGTGGCGGGAGCGGTGGCGGTA |
| | | GTGGTGGCGGCAGCGAAGGCGGTGGATCCGAAGGGGGCGGTTCCG |
| | | AGGGCGGTGGCTCGGAGGGTGGTGGCTCTGGCGGTGGCTCTGGCTC |
| | | CGGCGACTTCGATTACGAAAAAATGGCGAACGCGAACAAGGGTGCT |
| | | ATGACCGAGAACGCGGATGAAAACGCTCTGCAGAGCGATGCAAAG |
| | | GGTAAGCTGGACAGCGTTGCGACTGACTACGGCGCCGCAATTGACG |
| | | GCTTCATCGGCGACGTTAGCGGTTTGGCTAACGGCAATGGTGCAAC |
| | | GGGTGACTTTGCGGGCTCCAATAGCCAAATGGCACAGGTTGGTGAC |
| | | GGTGACAACAGCCCGCTGATGAATAACTTTCGTCAATACCTCCCAA |
| | | GCCTGCCGCAATCCGTGGAATGCCGTCCGTTCGTATTCTCCGCGGGT |
| | | AAGCCTTATGAGTTCTCAATCGACTGCGATAAGATTAACCTGTTTCG |
| | | TGGTGTTTTTGCGTTTCTGCTGTACGTGGCCACCTTTATGTATGTCTT |
| | | TAGCACCTTCGCCAACATTTTACGTAATAAAGAGAGCTAAggatccggct |
| | | gctaacaaagcccgaaaggaagctgagttggctgctgccaccgctgagcaataactagcata |
| | | accccttggggcctctaaacgggtcttgaggggttttttgACAAAGAGGGCGAGCGTTAA |
| 18 | NP_510894.1 | taatacgactcactatagggcgaattgggtaccgggcccagaccacaacggtttccctctag |
| | M13 gene IV | aaataattttgtttaactttaagaaggagatatacatATGAAACTATTAAATGTTATAAACT |
| | KTx00004.4 | TTGTATTCCTGATGTTTGTTTCGTCCTCATCTTTCGCCCAGGTTATTGAGATGAATA |
| | | ACTCCCCGCTGCGTGATTTTGTCACTTGGTATAGCAAGCAGAGCGGC |
| | | GAAAGCGTCATTGTTTCCCCGGATGTTAAAGGCACCGTTACCGTTTA |
| | | TAGCAGCGACGTTAAGCCGGAAAATCTGCGCAACTTCTTCATCAGC |
| | | GTGCTACGGGCGAATAACTTCGATATGGTTGGTAGCATCCCGAGCA |
| | | TTATTCAGAAATACAATCCGAATAACCAAGATTACATTGACGAACT |
| | | GCCGAGCTCGGACAACCAAGAGTATGATGATAACAGCGCTCCGTCC |
| | | GGTGGTTTTTTTGTACCGCAGAATGATAACGTAACGCAGACGTTTAA |
| | | AATCAACAATGTGCGCGCGAAAGATCTGATCCGTGTCGTGGAACTG |
| | | TTCGTGAAGTCTAACACGAGCAAGAGCTCTAACGTGTTGTCTATCGA |
| | | CGGCTCCAACTTATTAGTTGTTTCCGCGCCAAAAGACATCTTGGACA |
| | | ACCTGCCTCAGTTTCTGTCCACCGTGGACCTGCCGACCGACCAGATT |
| | | CTGATTGAGGGCTTGATTTTTGAAGTTCAGCAAGGTGACGCATTGGA |
| | | CTTCAGCTTCGCGGCGGGCAGCCAACGCGGTACAGTTGCTGGTGGC |
| | | GTGAACACCGATCGTTTGACCAGCGTTCTCTCTAGCGCGGGGGGTTC |
| | | ATTCGGCATCTTCAACGGTGATGTCTTGGGTCTGTCGGTTCGTGCCC |
| | | TGAAAACCAATTCTCACAGCAAGATCCTGTCCGTGCCGCGTATTCTG |
| | | ACCCTGTCAGGACAAAAAGGTTCCATCTCCGTGGGCCAAAACGTCC |
| | | CATTTATCACGGGTCGTGTGACCGGCGAGAGCGCGAACGTTAACAA |
| | | CCCGTTCCAGACCATCGAGCGCCAGAACGTGGGTATTAGCATGTCC |
| | | GTGTTCCCGGTAGCAATGGCAGGCGGTAATATTGTGCTGGACATCA |
| | | CGAGTAAGGCGGATTCGCTGAGCTCTTCGACTCAGGCAAGCGACGT |
| | | CATCACCAACCAAAGAAGCATCGCCACCACCGTTAATCTGCGTGAT |
| | | GGTCAAACCCTGCTGCTGGGCGGTTTGACTGACTACAAAAATACCTC |
| | | GCAAGACAGCGGCGTGCCGTTTTTGTCAAAGATCCCGCTGATAGGC |
| | | CTGTTGTTTAGCAGCCGTTCTGACAGTAATGAAGAGAGCACTCTTTA |
| | | CGTGCTGGTTAAGGCTACCATTggatccggctgctaacaaagcccgaaaggaagctgagtt |
| | | ggctgctgccaccgctgagcaataactagcataaccccttggggcctctaaacgggtcttga |
| | | ggggttttttgACAAAGAGGGCGAGCGTTAA |
| 19 | NP_510887.1 | taatacgactcactatagggcgaattgggtaccgggcccagaccacaacggtttccctctag |
| | M13 gene V | aaataattttgtttaactttaagaaggagatatacatATGATTAAAGTAGAAATAAAGCCCT |
| | KTx00004.5 | CACAAGCGCAGTTCACGACCCGTTCCGGTGTTAGCCGTCAGGGTAAGCCGTATAG |
| | | CTTAAATGAGCAGCTGTGCTACGTGGACCTGGGCAACGAGTACCCG |
| | | GTTTTGGTCAAAATCACCCTGGACGAAGGCCAACCGGCTTACGCAC |
| | | CGGGTCTGTATACCGTGCACCTGAGCTCTTTTAAAGTTGGTCAATTC |
| | | GGCTCGCTGATGATTGATCGTTTGCGCCTCGTGCCAGCGAAGTAAgga |
| | | tccggctgctaacaaagcccgaaaggaagctgagttggctgctgccaccgctgagcaataac |
| | | tagcataaccccttggggcctctaaacgggtcttgaggggttttttgACAAAGAGGGCGAGC |
| | | GTTAA |
| 20 | NP_510892.1 | taatacgactcactatagggcgaattgggtaccgggcccagaccacaacggtttccctctag |
| | M13 gene VI | aaataattttgtttaactttaagaaggagatatacatATGCCAGTACTGTTAGGAATACCCC |
| | KTx00004.6 | TATTGTTACGTTTTTTGGGTTTCTTGCTGGTCACCCTGTTCGGCTACTTGCTCACGT |
| | | TTCTGAAAAAAGGTTTTGGCAAGATCGCGATCGCCATCAGCCTGTTC |
| | | CTGGCACTGATTATCGGCCTGAATAGCATTCTGGTTGGTTACCTGAG |
| | | CGACATCTCTGCGCAGCTGCCGTCGGACTTCGTGCAAGGTGTTCAAC |
| | | TGATTCTGCCGTCCAACGCTTTGCCGTGCTTCTATGTTATTTTGTCCG |
| | | TGAAGGCGGCGATTTTCATCTTTGATGTAAAGCAGAAGATCGTGAG |
| | | CTATCTTGACTGGGATAAATAAggatccggctgctaacaaagcccgaaaggaagctgagtt |
| | | ggctgctgccaccgctgagcaataactagcataaccccttggggcctctaaacgggtcttga |
| | | ggggttttttgACAAAGAGGGCGAGCGTTAA |
| 21 | NP_510888.1 | taatacgactcactatagggcgaattgggtaccgggcccagaccacaacggtttccctctag |
| | M13 gene VII | aaataattttgtttaactttaagaaggagatatacatATGGAACAAGTAGCTGATTTTGACA |
| | KTx00004.7 | CAATATACCAGGCAATGATTCAGATTAGCGTTGTGTTGTGCTTCGCTCTGGGCATC |
| | | ATCGCGGGTGGTCAACGTTAAggatccggctgctaacaaagcccgaaaggaagctgagttg |
| | | gctgctgccaccgctgagcaataactagcataaccccttggggcctctaaacgggtcttgag |
| | | gggttttttgACAAAGAGGGCGAGCGTTAA |
| 22 | NP_510890.1 | taatacgactcactatagggcgaattgggtaccgggcccagaccacaacggtttccctctag |
| | M13 gene VIII | aaataattttgtttaactttaagaaggagatatacatATGAAAAAGTCACTAGTATTAAAAG |
| | KTx00004.8 | CTAGTGTCGCTGTGGCCACGTTGGTTCCGATGCTGTCGTTCGCCGCGGAAGGTGAT |
| | | GACCCGGCAAAAGCTGCGTTTAACAGCCTGCAGGCGTCTGCTACCG |
| | | AGTACATCGGCTATGCGTGGGCAATGGTGGTGGTTATTGTTGGTGCG |
| | | ACCATTGGCATCAAGCTGTTTAAAAAGTTCACCAGCAAGGCGTCCT |
| | | AAggatccggctgctaacaaagcccgaaaggaagctgagttggctgctgccaccgctgagca |
| | | ataactagcataaccccttggggcctctaaacgggtcttgaggggttttttgACAAAGAGGG |
| | | CGAGCGTTAA |
| 23 | NP_510889.1 | taatacgactcactatagggcgaattgggtaccgggcccagaccacaacggtttccctctag |
| | M13 gene IX | aaataattttgtttaactttaagaaggagatatacatATGAGTGTACTAGTTTATTCATTTG |
| | KTx00004.9 | CTTCTTTCGTGCTGGGTTGGTGCTTGCGCTCCGGCATCACCTACTTTACCCGTCTGA |
| | | TGGAAACCAGCAGCTAAggatccggctgctaacaaagcccgaaaggaagctgagttggctgct |
| | | gccaccgctgagcaataactagcataaccccttggggcctctaaacgggtcttgaggggttt |
| | | tttgACAAAGAGGGCGAGCGTTAA |
| 24 | Exemplary Template | acgcgccctgtagcggcgcattaagcgcggcgggtgtggtggttacgcgcagcgtgaccgct |
| | Vector | acacttgccagcgccctagcgcccgctcctttcgctttcttcccttcctttctcgccacgtt |
| | | cgccggctttccccgtcaagctctaaatcgggggctccctttagggttccgatttagtgctt |
| | | tacggcacctcgaccccaaaaaacttgatttgggtgatggttcacgtagtgggccatcgccc |
| | | tgatagacggtttttcgccctttgacgttggagtccacgttctttaatagtggactcttgtt |
| | | ccaaactggaacaacactcaaccctatctcgggacggatcgcttcatgtggcaggagaaaaa |
| | | aggctgcaccggtgcgtcagcagaatatgtgatacaggatatattccgcttcctcgctcact |
| | | gactcgctacgctcggtcgttcgactgcggcgagcggaaatggcttacgaacggggcggaga |
| | | tttcctggaagatgccaggaagatacttaacagggaagtgagagggccgcggcaaagccgtt |
| | | tttccataggctccgcccccctgacaagcatcacgaaatctgacgctcaaatcagtggtggc |
| | | gaaacccgacaggactataaagataccaggcgtttccccctggcggctccctcgtgcgctct |
| | | cctgttcctgcctttcggtttaccggtgtcattccgctgttatggccgcgtttgtctcattc |
| | | cacgcctgacactcagttccgggtaggcagttcgctccaagctggactgtatgcacgaaccc |
| | | cccgttcagtccgaccgctgcgccttatccggtaactatcgtcttgagtccaacccggaaag |
| | | acatgcaaaagcaccactggcagcagccactggtaattgatttagaggagttagtcttgaag |
| | | tcatgcgccggttaaggctaaactgaaaggacaagttttggtgactgcgctcctccaagcca |
| | | gttacctcggttcaaagagttggtagctcagagaaccttcgaaaaaccgccctgcaaggcgg |
| | | ttttttcgttttcagagcaagagattacgcgcagaccaaaacgatctcaagaagatcatctt |
| | | attaaggggtctgacgctcagtggaacgaaaactcacgttaagggattttggtcatgagatt |
| | | atcaaaaaggatcttcacctagatccttttaaattaaaaatgaagttttaaatcaatctaaa |
| | | gtatatatgagtaaacttggtctgacagttaccaatgcttaatcagtgaggcacctatctca |
| | | gcgatctgtctatttcgttcatccatagttgcctgactccccgtcgtgtagataactacgat |
| | | acgggagggcttaccatctggccccagtgctgcaatgataccgcgagacccacgctcaccgg |
| | | ctccagatttatcagcaataaaccagccagccgattcgagctcgccccggggatcgaccagt |
| | | tggtgattttgaacttttgctttgccacggaacggtctgcgttgtcgggaagatggtgatct |
| | | gatccttcaactcagcaaaagttcgatttattcaacaaagccgccgtcccgtcaagtcagcg |
| | | taatgctctgccagtgttacaaccaattaaccaattctgattagaaaaactcatcgagcatc |
| | | aaatgaaactgcaatttattcatatcaggattatcaataccatatttttgaaaaagccgttt |
| | | ctgtaatgaaggagaaaactcaccgaggcagttccataggatggcaagatcctggtatcggt |
| | | ctgcgattccgactcgtccaacatcaatacaacctattaatttcccctcgtcaaaaataagg |
| | | ttatcaagtgagaaatcaccatgagtgacgactgaatccggtgagaatggcaaaagcttatg |
| | | catttctttccagacttgttcaacaggccagccattacgctcgtcatcaaaatcactcgcat |
| | | caaccaaaccgttattcattcgtgattgcgcctgagcgagacgaaatacgcgatcgctgtta |
| | | aaaggacaattacaaacaggaatcgaatgcaaccggcgcaggaacactgccagcgcatcaac |
| | | aatattttcacctgaatcaggatattcttctaatacctggaatgctgttttcccggggatcg |
| | | cagtggtgagtaaccatgcatcatcaggagtacggataaaatgcttgatggtcggaagaggc |
| | | ataaattccgtcagccagtttagtctgaccatctcatctgtaacatcattggcaacgctacc |
| | | tttgccatgtttcagaaacaactctggcgcatcgggcttcccatacaatcgatagattgtcg |
| | | cacctgattgcccgacattatcgcgagcccatttatacccatataaatcagcatccatgttg |
| | | gaatttaatcgcggcctcgagcaagacgtttcccgttgaatatggctcataacaccccttgt |
| | | attactgtttatgtaagcagacagttttattgttcatgatgatatatttttatcttgtgcaa |
| | | tgtaacatcagagattttgagacacaacgtggctttccccccccccccctgcaggtctcggg |
| | | ctattcttttgatttataagggattttgccgatttcggcctattggttaaaaaatgagctga |
| | | tttaacaaaaatttaacgcgaattttaacaaaatattaacgtttacaatttaaatatttgct |
| | | tatacaatcttcctgtttttggggcttttctgattatcaaccggggtacatatgattgacat |
| | | gctagttttacgattaccgttcatgattctcttgtttgctccagactctcaggcaatgacct |
| | | gatagcctttgtagacctctcaaaaatagctaccctctccggcatgaatttatcagctagaa |
| | | cggttgaatatcatattgatggtgatttgactgtctccggcctttctcacccttttgaatct |
| | | ttacctacacattactcaggcattgcatttaaaatatatgagggttctaaaaatttttatcc |
| | | ttgcgttgaaataaaggcttctcccgcaaaagtattacagggtcataatgtttttggtacaa |
| | | ccgatttagctttatgctctgaggctttattgcttaattttgctaattctttgccttgcctg |
| | | tatgatttattggatgttaacgctactactattagtagaattgatgccaccttttcagctcg |
| | | cgccccaaatgaaaatatagctaaacaggttattgaccatttgcgaaatgtatctaatggtc |
| | | aaactaaatctactcgttcgcagaattgggaatcaactgttacatggaatgaaacttccaga |
| | | caccgtactttagttgcatatttaaaacatgttgagctacagcaccagattcagcaattaag |
| | | ctctaagccatccgcaaaaatgacctcttatcaaaaggagcaattaaaggtactctctaatc |
| | | ctgacctgttggagtttgcttccggtctggttcgctttgaagctcgaattaaaacgcgatat |
| | | ttgaagtctttcgggcttcctcttaatctttttgatgcaatccgctttgcttctgactataa |
| | | tagtcagggtaaagacctgatttttgatttatggtcattctcgttttctgaactgtttaaag |
| | | catttgagggggattcaatgaatatttatgacgattccgcagtattggacgctatccagtct |
| | | aaacattttactattaccccctctggcaaaacttcttttgcaaaagcctctcgctattttgg |
| | | tttttatcgtcgtctggtaaacgagggttatgatagtgttgctcttactatgcctcgtaatt |
| | | ccttttggcgttatgtatctgcattagttgaatgtggtattcctaaatctcaactgatgaat |
| | | ctttctacctgtaataatgttgttccgttagttcgttttattaacgtagatttttcttccca |
| | | acgtcctgactggtataatgagccagttcttaaaatcgcataaggtaattcacaatgattaa |
| | | agttgaaattaaaccatctcaagcccaatttactactcgttctggtgtttctcgtcagggca |
| | | agccttattcactgaatgagcagctttgttacgttgatttgggtaatgaatatccggttctt |
| | | gtcaagattactcttgatgaaggtcagccagcctatgcgcctggtctgtacaccgttcatct |
| | | gtcctctttcaaagttggtcagttcggttcccttatgattgaccgtctgcgcctcgttccgg |
| | | ctaagtaacatggagcaggtcgcggatttcgacacaatttatcaggcgatgatacaaatctc |
| | | cgttgtactttgtttcgcgcttggtataatcgctgggggtcaaagatgagtgttttagtgta |
| | | ttctttcgcctctttcgttttaggttggtgccttcgtagtggcattacgtattttacccgtt |
| | | taatggaaacttcctcatgaaaaagtctttagtcctcaaagcctctgtagccgttgctaccc |
| | | tcgttccgatgctgtctttcgctgctgagggtgacgatcccgcaaaagcggcctttaactcc |
| | | ctgcaagcctcagcgaccgaatatatcggttatgcgtgggcgatggttgttgtcattgtcgg |
| | | cgcaactatcggtatcaagctgtttaagaaattcacctcgaaagcaagctgataaaccgata |
| | | caattaaaggctccttttggagcctttttttttggagattttcaacgtgaaaaaattattat |
| | | tcgcaattcctttagttgttcctttctattctcactccgctgaaactgttgaaagttgttta |
| | | gcaaaaccccatacagaaaattcatttactaacgtctggaaagacgacaaaactttagatgt |
| | | tacgctaactatgagggttgtctgtggaatgctacaggcgttgtagtttgtactggtgacga |
| | | aactcagtgttacggtacatgggttcctattgggcttgctatccctgaaaatgagggtggtg |
| | | gctctgagggtggggttctgagggtggcggttctgagggtggcggtactaaacctcctgagt |
| | | acggtgatacacctattccgggctatacttatatcaaccctctcgacggcacttatccgcct |
| | | ggtactgagcaaaaccccgctaatcctaatccttctcttgaggagtctcagcctcttaatac |
| | | tttcatgtttcagaataataggttccgaaataggcagggggcattaactgtttatacgggca |
| | | ctgttactcaaggcactgaccccgttaaaacttattaccagtacactcctgtatcatcaaaa |
| | | gccatgtatgacgcttactggaacggtaaattcagagactgcgctttccattctggctttaa |
| | | tgaggatccattcgtttgtgaatatcaaggccaatcgtctgacctgcctcaacctcctgtca |
| | | atgctggcggcggctctggtggtggttctggtggcggctctgagggtggtggctctgagggt |
| | | ggcggttctgagggtggcggctctgagggaggcggttccggtggtggctctggttccggtga |
| | | ttttgattatgaaaagatggcaaacgctaataagggggctatgaccgaaaatgccgatgaaa |
| | | acgcgctacagtctgacgctaaaggcaaacttgattctgtcgctactgattacggtgctgct |
| | | atcgatggtttcattggtgacgtttccggccttgctaatggtaatggtgctactggtgattt |
| | | tgctggctctaattcccaaatggctcaagtcggtgacggtgataattcacctttaatgaata |
| | | atttccgtcaatatttaccttccctccctcaatcggttgaatgtcgcccttttgtctttagc |
| | | gctggtaaaccatatgaattttctattgattgtgacaaaataaacttattccgtggtgtctt |
| | | tgcgtttcttttatatgttgccacctttatgtatgtattttctacgtttgctaacatactgc |
| | | gtaataaggagtcttaatcatgccagttcttttgggtattccgttattattgcgtttcctcg |
| | | gtttccttctggtaactttgttcggctatctgcttacttttcttaaaaagggcttcggtaag |
| | | atagctattgctatttcattgtttcttgctcttattattgggcttaactcaattcttgtggg |
| | | ttatctctctgatattagcgctcaattaccctctgactttgttcagggtgttcagttaattc |
| | | tcccgtctaatgcgcttccctgtttttatgttattctctctgtaaaggctgctattttcatt |
| | | tttgacgttaaacaaaaaatcgtttcttatttggattgggataaataatatggctgtttatt |
| | | ttgtaactggcaaattaggctctggaaagacgctcgttagcgttggtaagattcaggataaa |
| | | attgtagctgggtgcaaaatagcaactaatcttgatttaaggcttcaaaacctcccgcaagt |
| | | cgggaggttcgctaaaacgcctcgcgttcttagaataccggataagccttctatatctgatt |
| | | tgcttgctattgggcgcggtaatgattcctacgatgaaaataaaaacggcttgcttgttctc |
| | | gatgagtgcggtacttggtttaatacccgttcttggaatgataaggaaagacagccgattat |
| | | tgattggtttctacatgctcgtaaattaggatgggatattatttttcttgttcaggacttat |
| | | ctattgttgataaacaggcgcgttctgcattagctgaacatgttgtttattgtcgtcgtctg |
| | | gacagaattactttaccttttgtcggtactttatattctcttattactggctcgaaaatgcc |
| | | ttctgcctaaattacatgttggcgttgttaaatatggcgattctcaattaagcccactgttg |
| | | agcgttggctttatactggtaagaatttgtataacgcatatgatactaaacaggctttttct |
| | | agtaattatgattccggtgtttattcttatttaacgccttatttatcacacggtcggtattt |
| | | caaaccattaaatttaggtcagaagatgaaattaactaaaatatatttgaaaaagttttctc |
| | | gcgttctttgtcttgcgattggatttgcatcagcatttacatatagttatataacccaacct |
| | | aagccggaggttaaaaaggtagtctctcagacctatgattttgataaattcactattgactc |
| | | ttctcagcgtcttaatctaagctatcgctatgttttcaaggattctaagggaaaattaatta |
| | | atagcgacgatttacagaagcaaggttattcactcacatatattgatttatgtactgtttcc |
| | | attaaaaaaggtaattcaaatgaaattgttaaatgtaattaattttgttttcttgatgtttg |
| | | tttcatcatcttcttttgctcaggtaattgaaatgaataattcgcctctgcgcgattttgta |
| | | acttggtattcaaagcaatcaggcgaatccgttattgtttctcccgatgtaaaaggtactgt |
| | | tactgtatattcatctgacgttaaacctgaaaatctacgcaatttctttatttctgttttac |
| | | gtgctaataattttgatatggttggttcaattccttccataattcagaagtataatccaaac |
| | | aatcaggattatattgatgaattgccatcatctgataatcaggaatatgatgataattccgc |
| | | tccttctggtggtttctttgttccgcaaaatgataatgttactcaaacttttaaaattaata |
| | | acgttcgggcaaaggatttaatacgagttgtcgaattgtttgtaaagtctaatacttctaaa |
| | | tcctcaaatgtattatctattgacggctctaatctattagttgttagtgcacctaaagatat |
| | | tttagataaccttcctcaattcctttctactgttgatttgccaactgaccagatattgattg |
| | | agggtttgatatttgaggttcagcaaggtgatgctttagatttttcatttgctgctggctct |
| | | cagcgtggcactgttgcaggcggtgttaatactgaccgcctcacctctgttttatcttctgc |
| | | tggtggttcgttcggtatttttaatggcgatgttttagggctatcagttcgcgcattaaaga |
| | | ctaatagccattcaaaaatattgtctgtgccacgtattcttacgctttcaggtcagaagggt |
| | | tctatctctgttggccagaatgtcccttttattactggtcgtgtgactggtgaatctgccaa |
| | | tgtaaataatccatttcagacgattgagcgtcaaaatgtaggtatttccatgagcgtttttc |
| | | ctgttgcaatggctggcggtaatattgttctggatattaccagcaaggccgatagtttgagt |
| | | tcttctactcaggcaagtgatgttattactaatcaaagaagtattgctacaacggttaattt |
| | | gcgtgatggacagactcttttactcggtggcctcactgattataaaaacacttctcaagatt |
| | | ctggcgtaccgttcctgtctaaaatccctttaatcggcctcctgtttagctcccgctctgat |
| | | tccaacgaggaaagcacgttatacgtgctcgtcaaagcaaccatagt |
| 25 | psc101 ori | gggctagtcaatgataattacatgtccttttcctttgagttgtgggtatctgtaaattctgc |
| | | tagacctttgctggaaaacttgtaaattctgctagaccctctgtaaattccgctagaccttt |
| | | gtgtgttttttttgtttatattcaagtggttataatttatagaataaagaaagaataaaaaa |
| | | agataaaaagaatagatcccagccctgtgtataactc |
| 26 | p15A ori | tttccataggctccgcccccctgacaagcatcacgaaatctgacgctcaaatcagtggtggc |
| | | gaaacccgacaggactataaagataccaggcgtttccccctggcggctccctcgtgcgctct |
| | | cctgttcctgcctttcggtttaccggtgtcattccgctgttatggccgcgtttgtctcattc |
| | | cacgcctgacactcagttccgggtaggcagttcgctccaagctggactgtatgcacgaaccc |
| | | cccgttc |
| 27 | pUC ori | ttgagatcctttttttctgcgcgtaatctgctgcttgcaaacaaaaaaaccaccgctaccag |
| | | cggtggtttgtttgccggatcaagagctaccaactctttttccgaaggtaactggcttcagc |
| | | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 28 | pUC inc1 ori (with | ttgagatcctttttttctgcgcgtaatctgctgcttgcaaacaaaaaaaccaccgctatcaa |
| | G62A relative to | cggtggtttgtttgccggatcaagagctaccaactctttttccgaaggtaactggcttcagc |
| | standard pUC) | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 29 | pUC inc2 ori (with | ttgagatcctttttttctgcgcgtaatctgctgcttgcaaacaaaaaaaccaccgctaccaa |
| | G62A relative to | cggtggtttgtttgccggatcaagagctatcaactctttttccgaaggtaactggcttcagc |
| | standard pUC) | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 30 | pUC inc1&2 ori | ttgagatcctttttttctgcgcgtaatctgctgcttgcaaacaaaaaaaccaccgctatcaa |
| | (with G62A relative | cggtggtttgtttgccggatcaagagctatcaactctttttccgaaggtaactggcttcagc |
| | to standard pUC) | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 31 | pUC inc1 ori | ttgagatcctttttttctgcgcgtaatctgctgcttgcaaacaaaaaaaccaccgctatcag |
| | | cggtggtttgtttgccggatcaagagctaccaactctttttccgaaggtaactggcttcagc |
| | | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 32 | pUC inc2 ori | ttgagatcctttttttctgcgcgtaatctgctgcttgcaaacaaaaaaaccaccgctaccag |
| | | cggtggtttgtttgccggatcaagagctatcaactctttttccgaaggtaactggcttcagc |
| | | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 33 | pUC inc1&2 ori | ttgagatcctttttttctgcgcgtaatctgctgcttgcaaacaaaaaaaccaccgctatcag |
| | | cggtggtttgtttgccggatcaagagctatcaactctttttccgaaggtaactggcttcagc |
| | | agagcgcagataccaaatactgtccttctagtgtagccgtagttaggccaccacttcaagaa |
| | | ctctgtagcaccgcctacatacctcgctctgctaatcctgttaccagtggctgctgccagtg |
| | | gcgataa |
| 34 | pyrF gene sequence | ATGACGTTAACTGCTTCATCTTCTTCCCGCGCTGTTACGAATTCTCC |
| | | TGTGGTTGTTGCCCTTGATTATCATAATCGTGATGACGCGCTGGCC |
| | | TTTGTCGACAAGATCGACCCACGCGATTGTCGTCTGAAGGTCGGC |
| | | AAAGAGATGTTTACATTGTTTGGGCCACAGTTTGTGCGCGAACTTC |
| | | AACAGCGTGGTTTTGATATCTTTCTTGACCTGAAATTCCACGATATC |
| | | CCCAACACTGCAGCGCACGCTGTCGCTGCTGCAGCTGACTTAGGC |
| | | GTGTGGATGGTGAATGTTCATGCCTCTGGTGGGGCGCGTATGATG |
| | | ACCGCAGCGCGTGAGGCACTGGTTCCGTTTGGCAAAGATGCACCG |
| | | CTTTTGATTGCTGTGACAGTGTTGACCAGCATGGAAGCCAGCGAC |
| | | CTGGTCGATCTTGGCATGACACTGTCACCTGCAGATTATGCAGAAC |
| | | GTCTGGCGGCACTGACGCAAAAATGTGGCCTTGATGGTGTGGTGT |
| | | GTTCTGCTCAGGAAGCTGTGCGCTTTAAACAGGTATTCGGTCAGGA |
| | | GTTCAAACTGGTTACGCCGGGCATTCGTCCGCAGGGGAGTGAAGC |
| | | TGGTGACCAGCGCCGCATTATGACGCCAGAACAGGCGTTGTCGGC |
| | | TGGTGTTGATTATATGGTGATTGGTCGCCCGGTAACGCAATCGGTA |
| | | GATCCAGCGCAGACGCTGAAAGCGATCAACGCCTCTTTACAGCGG |
| | | AGTGCATGA |

What is claimed is:

1. A production strain comprising a phagemid and at least two phage protein coding sequences integrated into the production strain genome, wherein the production strain can produce cssDNA upon introduction of the phagemid.

2. A circular single-stranded DNA (cssDNA) production system, comprising: at least two phage protein coding sequences integrated into the production strain genome; and a phagemid, wherein the production strain can produce cssDNA upon introduction of the phagemid into the production strain.

3. A circular single-stranded DNA (cssDNA) production system that does not contain a non-endogenous antibiotic resistance gene, comprising:
a production strain; and
a phagemid, wherein the phagemid comprises a packaging signal, a designed sequence, and at least one selectable sequence encoding one or more of an auxotrophic marker, an antitoxin, an RNA that inhibits the expression of a gene that would retard or stop growth of the bacterium if it were expressed in the absence of the RNA, a transcription factor repressor that inhibits the expression of a gene that would retard or stop growth of the bacterium if it were expressed in the absence of the transcription factor repressor, a transcriptional activator that activates a transcriptional repressor, a sequence that expresses a tRNA that associates with an unnatural amino acid that the production strain is engineered to require, and combinations thereof.

4. The production system of claim 3, wherein the production strain genome comprises at least two phage protein coding sequences integrated into the production strain genome.

5. The production strain or system according to any of claims 1-4, wherein the production strain is susceptible to infection by single-stranded, filamentous bacteriophages of the realm of Monodnaviria, and furthermore is a Gram negative bacteria of a family selected from the group consisting of Enterobacteriaceae, Pseudomonadaceae, Spirillaceae, Xanthomonadaceae, Clostridium, and Propionibacterium.

6. The production strain or system according to claim 5, wherein the production strain is susceptible to infection by a single stranded, filamentous bacteriophage selected from the group consisting of Ff, Fd, F1 and M13.

7. The production strain or system according to claim 6, wherein the production strain is susceptible to infection by the M13 bacteriophage.

8. The production strain or system of any one of claims 1-7, wherein the production strain is a strain of E. coli.

9. The production strain or system according to any one of claim 1-2 or 4-8, wherein the at least two phage protein coding sequences are differentially expressed as compared to each other.

10. The production strain or system according to any one of claim 1-2 or 4-8, wherein the at least two phage protein coding sequences are differentially expressed as compared to the native phage genome expression.

11. The production strain or system according to claim 9 or claim 10, wherein the at least two phage protein coding sequences encode at least two phage proteins selected from the group consisting of: p1, p2, p8, p10, and p11.

12. The production strain or system according to claim 11, wherein at least one of the at least two phage proteins are selected from the group consisting of: p3 and p5.

13. The production strain or system according to claim 12, wherein p3 activity is decreased as compared to native phage genome activity.

14. The production strain or system according to claim 12, wherein p5 activity is decreased as compared to native phage genome activity.

15. The production strain or system according to claim 1 or claim 2, wherein the at least two phage protein coding sequences are encoded by a filamentous bacteriophage of the realm of Monodnaviria.

16. The production strain or system of claim 15, wherein the filamentous bacteriophage is selected from a group consisting of: M13, Ff, Fd, Enterobacteria phage F1 [EF068134], Enterobacteria phage ID2, Enterobacteria phage NL95 [AF059243], Enterobacteria phage SP [X07489], Enterobacteria phage TW28, Enterobacteria phage Qbeta, Enterobacteria phage Qβ [AY099114], Enterobacteria phage M11 [AF059242], Enterobacteria phage ST, Enterobacteria phage TW18 [FJ483840], and Enterobacteria phage VK or a functional equivalent thereof.

17. The production strain or system according to claim 15 or claim 16, wherein the at least two phage protein coding sequences comprise at least 3, 4, 5, 6, 7, 8, 9, 10, or 11 phage protein coding sequences.

18. The production strain or system according to claim 1 or claim 2, wherein the at least two phage protein coding sequences include sequences encoding one or more bacteriophage M13 proteins selected from the group consisting of p1, p2, p3, p4, p5, p6, p7, p8, p9, p10 and p11.

19. The production strain or system according to claim 18, wherein the bacteriophage M13 protein sequences include M13 bacteriophage genes selected from the group consisting of I, II, III, IV, V, VI, VII, VIII, IX, X and XI.

20. The production strain or system according to claim 1 or claim 2, wherein the at least two phage protein coding sequences comprise at least three phage protein coding sequences.

21. The cssDNA production system according to claim 3, wherein the production strain comprises at least two genomically integrated phage protein coding sequences.

22. The cssDNA production system according to claim 21, wherein the at least two genomically integrated phage protein coding sequences are selected from sequences from one or more filamentous bacteriophages of the realm of Monodnaviria.

23. The cssDNA production system according to claim 22, wherein the one or more filamentous bacteriophages are selected from a group consisting of: M13, Ff, Fd, Enterobacteria phage F1 [EF068134], Enterobacteria phage ID2, Enterobacteria phage NL95 [AF059243], Enterobacteria phage SP [X07489], Enterobacteria phage TW28, Enterobacteria phage Qbeta, Enterobacteria phage Qβ [AY099114], Enterobacteria phage M11 [AF059242], Enterobacteria phage ST, Enterobacteria phage TW18 [FJ483840], Enterobacteria phage VK, and functional equivalents thereof.

24. The cssDNA production system according to claim 3, wherein the selectable sequence is an antitoxin sequence from a toxin/antitoxin system.

25. The ccsDNA production system according to claim 24, wherein the toxin/antitoxin system is selected from a group consisting of ccdB/ccdA, hokA/sokA, pemK/pemI, mazF/mazE, ChpBK ChpBI, relE/relB, parE/parD, hipA/hipB, and other toxin/antitoxin systems where the toxin is expressed from the host genome and the antitoxin is expressed from the phagemid.

26. The cssDNA production system according to claim 3, wherein the selectable sequence produces at least one RNA molecule that down regulates the expression of a counterselectable sequence selected from the group consisting of HSVtk, Ura3, tetA, sacB, rpsL, pheS, pheS*, pheS**, thyA, lacY, gata-1, ccdB, hokA, pemK, mazF, chpBK, relE, parE, hipA and other toxins.

27. The cssDNA production system according to claim 3, wherein the selectable sequence encodes a transcriptional factor repressor.

28. The cssDNA production system according to claim 27, wherein the transcriptional factor repressor is selected from the group consisting of tetR, araC, lacI, xylS, and other sequences that reduce the expression of a counterselectable marker or toxin.

29. The cssDNA production system according to claim 3, wherein the transcriptional activator is selected from the group consisting of araC and xylR.

30. The cssDNA production system according to claim 3, wherein the auxotrophic marker is selected from the group consisting of uracil adenine, cytosine, guanine, thymine, alanine, arginine, asparagine, aspartate, cysteine, glutamine, glutamate, glycine, histidine, Isoleucine, leucine, lysine, methionine, phenylalanine, proline, Serine, threonine, Tryptophan, tyrosine, valine, biotin, uridine-5′-monophosphate, pantothenate, xanthine, spermidine, para aminobenzoate, lipoate, nicotinamide riboside, nicotinamide mononucleotide, D-glucosamine, thiamin, shikimate, aminoethyl-phosphonate, beta alanine, s-methyl-metyhionine, ornithine, indole, indole acetic acid, L-threonine, L-threonine O-3-phosphate, nicotinate, ribosylnicotinamide, pyrimidine, agmatine, purine, and genes for the synthesis of other essential compounds expressed from the phagemid to complement a naturally occurring absence or synthetic deletion of a gene of the same or similar function in the host genome.

31. The production strain according to claim 1, further comprising the phagemid, wherein the phagemid comprises a packaging signal, a designed sequence, and at least one selectable sequence, optionally wherein the selectable sequence is selected from the group consisting of a sequence encoding an auxotrophic marker, an antitoxin, an RNA that inhibits the expression of a gene that would retard or stop growth of the bacterium if it were expressed in the absence of the RNA, a transcription factor repressor that inhibits the expression of a gene that would retard or stop growth of the bacterium if it were expressed in the absence of the transcription factor, a transcriptional activator that activates such a repressor, or a sequence that expresses a tRNA that associates with an unnatural amino acid that the production strain is engineered to require.

32. The production strain according to claim 31, wherein the auxotrophic marker is selected from the group consisting of uracil adenine, cytosine, guanine, thymine, alanine, arginine, asparagine, aspartate, cysteine, glutamine, glutamate, glycine, histidine, Isoleucine, leucine, lysine, methionine, phenylalanine, proline, Serine, threonine, Tryptophan, tyrosine, valine, biotin, uridine-5′-monophosphate, pantothenate, xanthine, spermidine, para aminobenzoate, lipoate, nicotinamide riboside, nicotinamide mononucleotide, D-glucosamine, thiamin, shikimate, aminoethyl-phosphonate, beta alanine, s-methyl-metyhionine, ornithine, indole, indole acetic acid, L-threonine, L-threonine O-3-phosphate, nicotinate, ribosylnicotinamide, pyrimidine, agmatine, purine, and genes for the synthesis of other essential compounds expressed from the phagemid to complement a naturally occurring absence or synthetic deletion of a gene of the same or similar function in the host genome.

33. The production strain or system according to claim 1 or claim 2, wherein the at least two phage protein coding sequences comprise at least two phage genes, wherein the at least two phage genes are operably linked to synthetic promoters to optimize expression for cssDNA production comprising one or more of canonical T7 promoters and mutant T7 promoters under the control of an inducible T7 polymerase, lacI, lacIq, araBAD, tet, temperature sensitive promoters, stress responsive promoters, quorums sensing promoters, light sensitive promoters and other inducible or repressible promoters.

34. The production strain or system according to claim 1 or claim 2, wherein the at least two phage protein coding sequences are integrated into the production strain genome at least two distinct loci.

35. The production strain or system according to claim 1 or claim 2, wherein at least one of the at least two phage protein coding sequences are altered from their endogenous sequence through random mutagenesis, rational design, assisted lab evolution, directed evolution or combinations thereof to create proteins that produce higher yields or more pure yields of cssDNA.

36. The cssDNA production system according to claim 2, wherein the phagemid expresses the tRNA necessary to translate a recoded codon of an unnatural amino acid, wherein the production strain cannot incorporate the unnatural amino acid into its proteins without the presence of the phagemid, and neither the phagemid nor the production strain can multiply or replicate without the presence of the unnatural amino acid.

37. The production strain or system according to claim 1 or claim 2, wherein at least one of the at least two phage protein coding sequences additionally comprises a tag.

38. The production strain according to claim 37, wherein the tag is selected from an affinity tag or a detection tag.

39. The production strain according to claim 38, wherein the detection tag is selected from a group consisting of: a fluorescent tag, a luminescent tag, a chromophoric tag, and another tag to enable rapid quantification of the number of phage particles in solution.

40. The production strain according to claim 38, wherein the affinity tag is selected from a group consisting of: biotin, his, myc, flag, CBP, GST, HA, HBH, MBP, S, V5 and another affinity tag to aid in purification of phage particles from production broth.

41. The production strain or system of any of claims 1-40, wherein the phagemid comprises a pUC origin of replication or a derivative thereof.

42. The production strain or system of any of claims 1-40, wherein the phagemid comprises a pST19, pDHA29, pDHA30, pDHK29, pDHK30, or runaway R1 origin of replication or derivative thereof.

43. The production strain or system of any of claims 1-42, wherein the phagemid comprises an inc1 mutation and/or an inc2 mutation, optionally wherein the phagemid comprises an inc1 mutation and an inc2 mutation.

44. The production strain or system of any of claims 1-43, wherein the phagemid comprises an inc3 mutation.

45. The production strain or system of any of claims 1-44, wherein the phagemid comprises an inc5 mutation.

46. The production strain or system of any of claims 1-45, wherein the phagemid copy number in production strain cells grown to late log phase is at least 1,000, optionally wherein the phagemid copy number in production strain cells grown to late log phase is at least 2,000, at least 4,000, at least 7,000, at least 8,000, or at least 15,000.

47. A circular single-stranded DNA (cssDNA) production system, comprising:
a production strain; and
a phagemid, wherein the phagemid comprises a packaging signal, a designed sequence, and at least one selectable sequence,
wherein the phagemid comprises a pUC origin of replication or a derivative thereof, and wherein the phagemid comprises an inc1 mutation and/or an inc2 mutation, optionally wherein the phagemid comprises an inc1 mutation and an inc2 mutation.

48. The production system of claim 47, wherein the production strain comprises at least two phage protein coding sequences integrated into the production strain genome.

49. A method of producing cssDNA, comprising;
culturing a production strain according to any of claim 1, 5-20, 31-35, or 37-46, in media;
introducing the phagemid; and
collecting the phage particles; and
separating the cssDNA.

50. The method according to claim 49, further comprising altering at least one of the at least two phage protein coding sequences.

51. The method of claim 50, wherein the method further comprises comparing the collected cssDNA to cssDNA collected from a parental strain from which the production strain was made to determine if the altered sequence improves titer or quality of cssDNA produced.

52. The method according to claim 49, wherein the production strain further comprises at least one phage protein comprising a tag.

53. The method according to claim 52, wherein the tag is used to separate the phage.

54. A method of producing cssDNA, comprising;
culturing cssDNA production system according to any of claims 2-48, in media;
collecting the phage particles; and
separating the cssDNA.

55. The method according to claim 54, wherein the production strain further comprises at least one phage protein comprising a tag.

56. The method according to claim 55, wherein the tag is used to separate the phage.

57. The production system of claim 3 or the method of claim 54, wherein the production strain comprises a helper plasmid derived from helper phage M13KO7 by removing the F1 origin and the packaging signal, optionally wherein the helper plasmid copy number in production strain cells grown to late log phase is at least 1,000, optionally wherein the helper plasmid copy number in production strain cells grown to late log phase is at least 2,000, at least 4,000, at least 7,000, at least 8,000, or at least 15,000.

58. The production strain, system or method of claim 57, wherein the helper plasmid comprises a pUC origin of replication.

59. The production strain, system, or method of any preceding claim, wherein the production strain is an engineered variant of a BW25113 strain.

60. The production strain, system or method according to any of claims 33-59, wherein a nucleotide sequence encoding the T7 polymerase is integrated into the production strain genome at an endA locus.

61. The production strain, system or method of claim 60, wherein the nucleotide sequence encoding the T7 polymerase is under control of an IPTG-inducible lac promoter.

62. The production strain, system, or method of any preceding claim, wherein the production strain comprises M13 genes II, V, VII, VIII, and IX integrated into the production strain genome at an intA locus.

63. The production strain, system, or method of any preceding claim, wherein the production strain comprises M13 genes III, VI, I, and IV integrated into the production strain genome at an intZ locus.

64. The production strain, system, or method of any preceding claim, further comprising M13 gene II integrated into the production strain genome at a mazF locus.

65. The production strain, system, or method of any preceding claim, further comprising M13 gene VIII integrated into the production strain genome at a mazF locus.
